(19) United States
(12) Patent Application Publication
(10) Pub. No.: US 2025/0304634 A1
(43) Pub. Date: Oct. 2, 2025

(54) PROTEINS AND PRODUCTS COMPRISING THE SAME

(71) Applicant: AMAI PROTEINS LTD., Rehovot (IL)

(72) Inventors: Naama KOPELMAN, Givatayim (IL); IIan SAMISH, Ness Ziona (IL); Lior BEN-ASAF, Kiryat Ono (IL); Adva YEHESKEL, Atzmon-Segev (IL); Shmuel MARKO, DoarNa Lev HaSharon (IL); Inbar ZUKER, Ness Ziona (IL); Lihi ELIASPUR, Nordiya (IL); Noy ALMASSI, Rishon LeZion (IL); Marina KASHDAN, Ashdod (IL)

(73) Assignee: AMAI PROTEINS LTD., Rehovot (IL)

(21) Appl. No.: 18/849,901

(22) PCT Filed: Mar. 23, 2023
(86) PCT No.: PCT/IL2023/050310

§ 371 Date: Sep. 23, 2024

Related U.S. Application Data
(60) Provisional application No. 63/323,297, filed on Mar. 24, 2022.
(60) Provisional application No. 63/367,298, filed on Jun. 29, 2022.

(51) Int. Cl. C07K 14/43; A23L 2/56; A23L 2/60; A23L 27/00; A23L 27/30; A23L 33/18

(52) U.S. Cl. CPC C07K 14/43; A23L 2/56; A23L 2/60; A23L 27/31; A23L 27/84; A23L 27/88; A23L 33/18

(57) ABSTRACT

The present disclosure relates to a modified protein comprising an amino acid sequence that has one or more amino acids modifications from a reference protein.

### FIELD OF THE INVENTION

The present invention relates to novel proteins and food products comprising the same.

### BACKGROUND ART

References considered to be relevant as background to the presently disclosed subject matter are listed below:
GB2123672
WO8402450
WO2019215730
Liu Yang, et al. Chemical Senses, 2019, Vol 44, 607-614 and
CN109627307

Acknowledgement of the above references herein is not to be inferred as meaning that these are in any way relevant to the patentability of the presently disclosed subject matter.

### BACKGROUND

Sugar alternatives are receiving increased attention due to increased disease awareness as a result of sugar overconsumption as the underlying cause of the metabolic syndrome.

Sweet proteins were suggested as an alternative for sugar.

GB2123672 describes sweet proteins, such as Thaumatin and Monellin, and an incorporated weakly acidic polysaccharide gum, optionally together with a food acid or bulking agent, in various beverages, mouthwashes, or as a pharmaceutical base.

WO8402450 describes the application of Thaumatin or Monellin to the surface of a chewing gum composition comprising gum base, sweetener, and flavoring.

WO2019215730 discloses modified proteins with improved food-related properties.

Liu Yang, et al. Chemical Senses, 2019, Vol 44, 607-614 and CN109627307 describe modifications in the loop region of Single-Chain Monellin (MNEI).

### GENERAL DESCRIPTION

In accordance with some aspects, it is provided a modified protein comprising an amino acid sequence that comprise at least one amino acid modification in in at least one of (i) an alpha helix of the reference protein, (ii) a core of the reference protein, (iii) a sweet loop of the reference protein, (iv) a linker region of the reference protein or (v) any combination thereof, wherein the reference protein has an amino acid sequence selected from the group consisting of SEQ ID NO:8, SEQ ID NO:9 and SEQ ID NO:10.

In accordance with some aspects, it is provided a modified protein comprising an amino acid sequence that has amino acid deletions in at least three amino acids located between amino acid T46 and amino acid 156, as compared to a reference protein, wherein said reference protein has an amino acid sequence set forth in SEQ ID NO:8.

In accordance with some aspects, it is provided a food product comprising the protein of the present disclosure.

### EMBODIMENTS

Some embodiments of this disclosure will now be described in the following numbered paragraph. The following description intends to add on the above general description and not limit it in any manner.

1. A modified protein comprising an amino acid sequence that comprise at least one amino acid modification in in at least one of (i) an alpha helix of the reference protein, (ii) a core of the reference protein, (iii) a sweet loop of the reference protein, (iv) a linker region of the reference protein or (v) any combination thereof, wherein the reference protein has an amino acid sequence selected from the group consisting of SEQ ID NO:8, SEQ ID NO:9 and SEQ ID NO:10.
2. The modified protein of Embodiment No. 1, having an energy lower than about −315 given in REU.
3. The modified protein of Embodiment No. 2, having an energy lower than about −321 given in REU.
4. The modified protein of any one of Embodiment Nos. 1 to 3, comprises an amino acid sequence 90% to 98% identical to the reference protein.
5. A modified protein comprising an amino acid sequence that has amino acid deletions in at least three amino acids located between amino acid T46 and amino acid 156, as compared to a reference protein, wherein the reference protein has an amino acid sequence selected from the group consisting of SEQ ID NO:8, SEQ ID NO:9 and SEQ ID NO:10.
6. The modified protein of any one of Embodiment Nos. 1 to 5, comprising amino acid deletions in at least amino acid E50, F52 and R53 as compared to a reference protein.
7. The modified protein of any one of Embodiment Nos. 1 to 6, wherein the reference protein is as set forth in SEQ ID NO:8.
8. The modified protein of any one of Embodiment Nos. 1 to 7, comprising at least one amino acid substitution as compared to the reference protein.
9. The modified protein of any one of Embodiment Nos. 1 to 7, comprising at least five amino acid substitutions as compared to the reference protein.
10. The modified protein of Embodiment No. 9, comprising substitutions in amino acids E2, E23 and Y65 as compared to the reference protein, wherein the reference protein is as set forth in SEQ ID NO:8.
11. The modified protein of any one of Embodiment Nos. 1 to 10, wherein the reference protein is as set forth in SEQ ID NO:9.
12. The modified protein of any one of Embodiment Nos. 9 to 11, comprising substitution in amino acid L70 as compared to the reference protein.
13. The modified protein of any one of Embodiment Nos. 9 or 12, comprising substitution in amino acids E2, E23, Y65 and L70 as compared to the reference protein.
14. The modified protein of any one of Embodiment Nos. 1 to 13, wherein the reference protein is as set forth in SEQ ID NO:10.
15. The modified protein of any one of Embodiment Nos. 9 to 14, comprising an amino acid substitution in at least one amino acid selected from the group consisting of E4, T12, A19, V20, K25, 126, Q28, R31, T33, N35, C41, Q61, V64, D68, A73, 175, R84 and F89 as compared to said reference protein.
16. The modified protein of Embodiment No. 15, comprising an amino acid substitution in at least one amino acid selected from the group consisting of Q28, C41 and Q68 as compared to said reference protein.
17. The modified protein of Embodiment No. 15, comprising an amino acid substitution in at least one amino acid selected from the group consisting of E4, A19, V20, K25, 126, T33, N35 A73, and R84 as compared to said reference protein.
18. The modified protein of Embodiment No. 15, comprising an amino acid substitution in at least one amino acid selected from the group consisting of T12, R31, Q61, V64, 175 and F89 and as compared to said reference protein.
19. The modified protein of Embodiment No. 15, comprising an amino acid substitution in at least one amino acid selected from the group consisting of A19, V20, K25, 126, Q28, T33, C41 and D68 as compared to said reference protein.
20. The modified protein of Embodiment No. 15, comprising an amino acid substitution in at least one amino acid selected from the group consisting of A19, V20, K25, 126, Q28, T33, C41 and D68 as compared to said reference protein.
21. The modified protein of Embodiment No. 9 or 10, comprising substitutions in at least one amino acid that (i) stabilizes alpha helix structure, (ii) reduce aggregation, (iii) affect core repacking, (iv) affect electrostatics, or (v) any combination thereof of the reference protein, the modified protein or both.
22. The modified protein of Embodiment No. 21, comprising substitutions in at least one amino acid that affect core repacking of the reference protein.
23. The modified protein of Embodiment No. 9 or 10, comprising substitutions in at least one amino acid located in (i) alpha helix, (ii) core, (iii) sweet loop or (iv) any combination thereof of the reference protein.
24. The modified protein of Embodiment No. 23, composing substitution in at least one amino acid that are located in the core of the reference protein.
25. The modified protein of Embodiment No. 15 or 24, comprising at least one amino acid substitution in at least one amino acid residue selected from the group consisting of T12, C41, A19, V20, A73, 175, F89, G16, L32, V37, L62 and V64 as compared to the reference protein.
26. The modified protein of Embodiment No. 25, comprising at least one amino acid substitution in at least one amino acid residue selected from the group consisting of T12, C41, A19, V20, A73, 175 and F89 as compared to the reference protein.
27. The modified protein of Embodiment No. 26, comprising at least one amino acid substitution in at least one amino acid residue selected from the group consisting of A19 and V20 as compared to the reference protein.
28. The modified protein of Embodiment No. 21, comprising substitutions in at least one amino acid that stabilizes alpha helix structure of the reference protein.
29. The modified protein of Embodiment No. 15 or 28, comprising substitution in at least one amino acid K25, 126, Q28 or any combination thereof as compared to the reference protein.
30. The modified protein of Embodiment No. 23, composing substitution in at least one amino acid that are located in the sweet loop of the reference protein.
31. The modified protein of Embodiment No. 15 or 30, comprising substitution in at least one amino acid selected from the group consisting of T33 and D68 as compared to the reference protein.
32. The modified protein of Embodiment No. 21, comprising substitutions in at least one amino acid that reduces aggregation of the reference protein, the modified protein or both.
33. The modified protein of Embodiment No. 15 or 32, comprising substitution in amino acid C41 as compared to the reference protein.
34. The modified protein of Embodiment 21, comprising substitutions in at least one amino acid that affect electrostatics of the reference protein.
35. The modified protein of Embodiment No. 15 or 34, comprising substitution in at least one amino acid residue T33, E4 or any combinations thereof as compared to the reference protein.
36. The modified protein of any one of Embodiment Nos. 1 to 35, comprising an amino acid substitution in at least amino acid E4 as compared to the reference protein.
37. The modified protein of any one of Embodiment Nos. 1 to 35, comprising an amino acid substitution in at least amino acid T12 as compared to the reference protein.
38. The modified protein of any one of Embodiment Nos. 1 to 35, comprising an amino acid substitution in at least amino acid A19 as compared to the reference protein.
39. The modified protein of any one of Embodiment Nos. 1 to 35, comprising an amino acid substitution in at least amino acid V20 as compared to the reference protein.
40. The modified protein of any one of Embodiment Nos. 1 to 35, comprising an amino acid substitution in at least amino acid K25 as compared to the reference protein.
41. The modified protein of any one of Embodiment Nos. 1 to 35, comprising an amino acid substitution in at least amino acid 126 as compared to the reference protein.
42. The modified protein of any one of Embodiment Nos. 1 to 35, comprising an amino acid substitution in at least amino acid Q28 as compared to the reference protein.
43. The modified protein of any one of Embodiment Nos. 1 to 35, comprising an amino acid substitution in at least amino acid R31 as compared to the reference protein.
44. The modified protein of any one of Embodiment Nos. 1 to 35, comprising an amino acid substitution in at least amino acid T33 as compared to the reference protein.
45. The modified protein of any one of Embodiment Nos. 1 to 35, comprising an amino acid substitution in at least amino acid N35 as compared to the reference protein.
46. The modified protein of any one of Embodiment Nos. 1 to 35, comprising an amino acid substitution in at least amino acid C41 as compared to the reference protein.
47. The modified protein of any one of Embodiment Nos. 1 to 35, comprising an amino acid substitution in at least amino acid Q61 as compared to the reference protein.
48. The modified protein of any one of Embodiment Nos. 1 to 35, comprising an amino acid substitution in at least amino acid V64 as compared to the reference protein.
49. The modified protein of any one of Embodiment Nos. 1 to 35, comprising an amino acid substitution in at least amino acid D68 as compared to the reference protein.
50. The modified protein of any one of Embodiment Nos. 1 to 35, comprising an amino acid substitution in at least amino acid A73 as compared to the reference protein.
51. The modified protein of any one of Embodiment Nos. 1 to 35, comprising an amino acid substitution in at least amino acid 175 as compared to the reference protein.
52. The modified protein of any one of Embodiment Nos. 1 to 35, comprising an amino acid substitution in at least amino acid R84 as compared to the reference protein.
53. The modified protein of any one of Embodiment Nos. 1 to 35, comprising an amino acid substitution in at least amino acid F89 as compared to the reference protein.
54. The modified protein of any one of Embodiment Nos. 1 to 35, comprising an amino acid substitution in amino acid Q28 and an amino acid substitution in at least amino acid selected from the group consisting of V20, K25, 126 and R31 as compared to the reference protein.
55. The modified protein of any one of Embodiment Nos. 1 to 35, comprising amino acid substitutions in amino acids Q28 and V20 as compared to the reference protein.
56. The modified protein of any one of Embodiment Nos. 1 to 35, comprising amino acid substitutions in amino acids Q28 and K25, as compared to the reference protein.
57. The modified protein of any one of Embodiment Nos. 1 to 35, comprising amino acid substitutions in amino acids Q28 and R31, as compared to the reference protein.
58. The modified protein of any one of Embodiment Nos. 1 to 35, comprising an amino acid substitution in amino acid Q28 and 126 as compared to the reference protein.
59. The modified protein of any one of Embodiment Nos. 1 to 35, comprising an amino acid substitution in amino acid D68 and in at least amino acid selected from the group consisting of V20, R31, R84 as compared to the reference protein.
60. The modified protein of any one of Embodiment Nos. 1 to 35, comprising an amino acid substitution in amino acids V20 and D68 as compared to the reference protein.
61. The modified protein of any one of Embodiment Nos. 1 to 35, comprising an amino acid substitution in amino acids D68 and R84 as compared to the reference protein.
62. The modified protein of any one of Embodiment Nos. 1 to 35, comprising an amino acid substitution in amino acids D68 and R31 as compared to the reference protein.
63. The modified protein of any one of Embodiment Nos. 1 to 35, comprising an amino acid substitution in amino acids R84 and R31 as compared to the reference protein.
64. The modified protein of any one of Embodiment Nos. 1 to 35, comprising an amino acid substitution in amino acids A73 and F89 as compared to the reference protein.
65. The modified protein of any one of Embodiment Nos. 1 to 35, comprising an amino acid substitution in amino acids C41 and T12 as compared to the reference protein.
66. The modified protein of any one of Embodiment Nos. 1 to 35, comprising an amino substitution in amino acid V20 and in at least amino acid selected from the group consisting of K25, V64 and D68 as compared to the reference protein.
67. The modified protein of any one of Embodiment Nos. 1 to 35, comprising an amino acid substitution in amino acids V20 and V64 as compared to the reference protein.
68. The modified protein of any one of Embodiment Nos. 1 to 35, comprising an amino acid substitution in amino acids V20 and K25 as compared to the reference protein.
69. The modified protein of any one of Embodiment Nos. 1 to 35, comprising an amino acid substitution in amino acids V20 and C41 and A73 and 175 and F89 as compared to the reference protein.
70. The modified protein of Embodiment No. 10, comprising at least the amino acids substitutions selected from the group consisting of E2N, E23A and Y65R.
71. The modified protein of Embodiment No. 10, 13 or 70, comprising at least the amino acids substitutions selected from the group consisting of E2N, E23A, Y65R and L70I.
72. The modified protein of Embodiment No. 10, comprising at least the amino acids substitutions selected from the group consisting of E2N, E23V and Y65K.
73. The modified protein of Embodiment No. 10, 13 or 72, comprising at least the amino substitutions selected from the group consisting of E2N, E23V, Y65K and L70I.
74. The modified protein of any one of Embodiment Nos. 1 to 73, comprising at least one amino acid substitution selected from the group consisting of E4Q, T12V, A19V, V20I, K25R, 126T, 126V, 126W, Q28K, Q28R, R31T, T33R, N35T, C41T, C41V, C41A, Q61N, V64I, V64L, D68Q, D68N, A73V, A73F, I75L, R84L, R84K, and F89M as compared to the reference protein, wherein the reference protein selected from the group consisting of SEQ ID NO:8, SEQ ID NO:9 and SEQ ID NO:10.
75. The modified protein of any one of Embodiment Nos. 1 to 73, comprising an amino acid substitution in at least one amino acid selected from the group consisting of Q28K, Q28R, C41T, C41V, C41A, D68Q and D68N, as compared to said reference protein.
76. The modified protein of Embodiment Nos. 1 to 73, comprising an amino acid substitution in at least one amino acid selected from the group consisting of E4Q, A19V, V20I, K25R, I26T, I26V, I26W, T33R, N35T, A73V, A73F, and R84L, R84K, as compared to said reference protein.
77. The modified protein of Embodiment Nos. 1 to 73, comprising an amino acid substitution in at least one amino acid selected from the group consisting of T12V, R31T, Q61N, V64I, V64L, I75L, and F89M and as compared to said reference protein.
78. The modified protein of any one of Embodiment Nos. 1 to 73, comprising an amino acid substitution in at least amino acid E4Q as compared to the reference protein.
79. The modified protein of any one of Embodiment Nos. 1 to 73, comprising an amino acid substitution in at least amino acid T12V as compared to the reference protein.
80. The modified protein of any one of Embodiment Nos. 1 to 73, comprising an amino acid substitution in at least amino acid A19V as compared to the reference protein.
81. The modified protein of any one of Embodiment Nos. 1 to 73, comprising an amino acid substitution in at least amino acid V20I as compared to the reference protein.
82. The modified protein of any one of Embodiment Nos. 1 to 73, comprising an amino acid substitution in at least amino acid K25R as compared to the reference protein.
83. The modified protein of any one of Embodiment Nos. 1 to 73, comprising an amino acid substitution in at least amino acid I26W as compared to the reference protein.
84. The modified protein of any one of Embodiment Nos. 1 to 73, comprising an amino acid substitution in at least amino acid I26T as compared to the reference protein.
85. The modified protein of any one of Embodiment Nos. 1 to 73, comprising an amino acid substitution in at least amino acid I26V as compared to the reference protein.
86. The modified protein of any one of Embodiment Nos. 1 to 73, comprising an amino acid substitution in at least amino acid Q28K as compared to the reference protein.
87. The modified protein of any one of Embodiment Nos. 1 to 73, comprising an amino acid substitution in at least amino acid Q28R as compared to the reference protein.
88. The modified protein of any one of Embodiment Nos. 1 to 73, comprising an amino acid substitution in at least amino acid R3IT as compared to the reference protein.
89. The modified protein of any one of Embodiment Nos. 1 to 73, comprising an amino acid substitution in at least amino acid T33R as compared to the reference protein.
90. The modified protein of any one of Embodiment Nos. 1 to 73, comprising an amino acid substitution in at least amino acid N35T as compared to the reference protein.
91. The modified protein of any one of Embodiment Nos. 1 to 73, comprising an amino acid substitution in at least amino acid C41T as compared to the reference protein.
92. The modified protein of any one of Embodiment Nos. 1 to 73, comprising an amino acid substitution in at least amino acid C41A as compared to the reference protein.
93. The modified protein of any one of Embodiment Nos. 1 to 73, comprising an amino acid substitution in at least amino acid C41V as compared to the reference protein.
94. The modified protein of any one of Embodiment Nos. 1 to 73, comprising an amino acid substitution in at least amino acid Q61N as compared to the reference protein.
95. The modified protein of any one of Embodiment Nos. 1 to 73, comprising an amino acid substitution in at least amino acid V64I as compared to the reference protein.
96. The modified protein of any one of Embodiment Nos. 1 to 73, comprising an amino acid substitution in at least amino acid V64L as compared to the reference protein.
97. The modified protein of any one of Embodiment Nos. 1 to 73, comprising an amino acid substitution in at least amino acid D68N as compared to the reference protein.
98. The modified protein of any one of Embodiment Nos. 1 to 73, comprising an amino acid substitution in at least amino acid D68Q as compared to the reference protein.
99. The modified protein of any one of Embodiment Nos. 1 to 73, comprising an amino acid substitution in at least amino acid A73V as compared to the reference protein.
100. The modified protein of any one of Embodiment Nos. 1 to 73, comprising an amino acid substitution in at least amino acid A73F as compared to the reference protein.
101. The modified protein of any one of Embodiment Nos. 1 to 73, comprising an amino acid substitution in at least amino acid I75L as compared to the reference protein.
102. The modified protein of any one of Embodiment Nos. 1 to 73, comprising an amino acid substitution in at least amino acid R84K as compared to the reference protein.
103. The modified protein of any one of Embodiment Nos. 1 to 73, comprising an amino acid substitution in at least amino acid R84L as compared to the reference protein.
104. The modified protein of any one of Embodiment Nos. 1 to 73, comprising an amino acid substitution in at least amino acid F89M as compared to the reference protein.
105. The modified protein of any one of Embodiment Nos. 1 to 104, comprising an amino acid sequence that is between about 90% and about 99% identical to amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:12, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:50, SEQ ID NO:51, SEQ ID NO:52, SEQ ID NO:54, SEQ ID NO:56, SEQ ID NO:57, SEQ ID NO:58, SEQ ID NO:59, SEQ ID NO:60, SEQ ID NO:63, SEQ ID NO:64, SEQ ID NO:65, SEQ ID NO:66, SEQ ID NO:67, SEQ ID NO:68, SEQ ID NO:69 and SEQ ID NO:71.
106. The modified protein of any one of Embodiment Nos. 1 to 104, comprising an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:12, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:50, SEQ ID NO:51, SEQ ID NO:52, SEQ ID NO:54, SEQ ID NO:56, SEQ ID NO:57, SEQ ID NO:58, SEQ ID NO:59, SEQ ID NO:60, SEQ ID NO:63, SEQ ID NO:64, SEQ ID NO:65, SEQ ID NO:66, SEQ ID NO:67, SEQ ID NO:68, SEQ ID NO:69 and SEQ ID NO:71.
107. The modified protein of any one of Embodiment Nos. 1 to 106, comprising at least one amino acid sequence shown in Table 20.
108. The modified protein of any one of Embodiment Nos. 1 to 106 having an improved sweetness as compared to SEQ ID NO:8.
109. The modified protein of any one of Embodiment Nos. 1 to 106 having an improved sweetness as compared to SEQ ID NO:9.
110. The modified protein of any one of Embodiment Nos. 1 to 106 having an improved sweetness as compared to SEQ ID NO:10.
111. The modified protein of any one of Embodiment Nos. 1 to 106 having an improved sweetness as compared to SEQ ID NO:7.
112. The modified protein of any one of Embodiment Nos. 1 to 106 having an improved heat treatment as compared to SEQ ID NO:8.
113. The modified protein of any one of Embodiment Nos. 1 to 106 having an improved heat treatment as compared to SEQ ID NO:9.
114. The modified protein of any one of Embodiment Nos. 1 to 106 having an improved heat treatment as compared to SEQ ID NO:10.
115. The modified protein of any one of Embodiment Nos. 1 to 106 having an improved heat treatment as compared to SEQ ID NO:7.
116. The modified protein of any one of Embodiment Nos. 1 to 106 having an improved Tm as compared to SEQ ID NO:8.
117. The modified protein of any one of Embodiment Nos. 1 to 106 having an improved Tm as compared to SEQ ID NO:9.
118. The modified protein of any one of Embodiment Nos. 1 to 106 having an improved Tm as compared to SEQ ID NO:10.
119. The modified protein of any one of Embodiment Nos. 1 to 106 having an improved Tm as compared to SEQ ID NO:7.
120. A modified protein selected from the group consisting of DM65, DM57, DM43, DM96, DM87, DM77, DM150, DM151, DM46, DM66, DM69, DM41, DM72, DM42, DM70, DM85, DM75 and DM47.
121. A food product comprising any one of Embodiment Nos. 1 to 120.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a spider graph showing that an energy bar with a 40% reduction of added sugar is less sweet compared to an energy bar with DM proteins prototype.

FIG. 2 is a spider graph showing that Marzipan with sweet protein DM31 (70% reduction of added sugar) is sweeter than Marzipan with 70% reduction of added sugar.

FIG. 3 is a graph showing the stability of DM31 in a buffer citrate. Y-axis is sweetness intensity on a scale of 0-100, while 100 define as 8° Bx equivalent. The sweetness intensity of DM31 is stable after 12 weeks both at 21° C. and 32° C. The black line on the graphs indicates degradation of 25% in sweetness intensity.

FIG. 4 is a histogram showing that sweetness intensity of DM31 in rice flour with 40% moisture content is stable at 60° C. for 24 hours in closed jars. Y-axis is sweetness intensity on a scale of 0-100, while 100 define as 8° Bx equivalent.

FIG. 5 is histograms showing the heat stability of DM31 in a powder form at high temperatures. The sweetness intensity of DM31 in a powder form is stable up to 120° C. for 5 minutes.

FIGS. 6A-6C show the 126 and T26 hydrogen bonds, before (FIG. 6A) and after (FIGS. 6B and 6C) the substitution I26T, hydrogen bonds of the backbone are represented by dashed yellow lines, FIGS. 6B and 6C show the two optional hydrogen bonds which are formed following the substitution—either between T26 to E22 backbone or between T26 to Q28.

FIG. 7 shows the duration of specific hydrogen bonds, based on measured distance between acceptor and donor throughout the molecular dynamics simulations. Fractions are shown for both DM31 and DM42, for comparison purposes. The larger the bar, the more frequently the bond is detected during simulations. A distance of 3.5 Å was used as a threshold. Importantly, after the substitution I26T of DM42, the bonds between residue 26 and residues 22 & 23, supporting the helix, are satisfied and protected, and are thus present for a larger fraction of time.

FIG. 8 demonstrates the K25 hydrogen bonds before substitution (i.e. in MNEI). Hydrogen bonds of the backbone are represented by dashed yellow lines. The hydrogen bonds between K25 and E22 are marked by the upper (backbone-backbone) and lower (sidechain-sidechain) arrows. These bonds are of importance to the stability of the helix, and further stabilized by the substitution in DM43, K25R.

FIGS. 9A and 9B shows C41 in sticks (A), vs. T41 in sticks (B). An inner cavity is shown as a purple shape. The substitution C41T leads to a decreased cavity volume within the proteins' core.

FIG. 10 is a spider graph showing that granola with 70% sugar reduction and DM31 sweet protein is sweeter, compared to granola with 70% sugar reduction and no-protein.

FIG. 11 is a spider graph showing that the peanut butter spread with 75% sugar reduction combined with Amai sweet protein is sweeter, compared to the peanut butter spread with 75% sugar reduction and no-protein addition.

FIG. 12 Average RMSF (Root Mean Square Fluctuations) plots of Helix Capping variants. DM42 (DM31+I26T), DM43 (DM31+K25R), DM65 (DM31+Q28K), DM115 (DM31+I26W+Q28E), DM116 (DM31+I26W+Q28K), DM144 (DM31+I26W). The results were obtained using GROMACS 2022.1.

FIG. 13 Average RMSF (Root Mean Square Fluctuations) plots of Core Repacking variants. DM96 (DM31+V20I), DM77 (DM31+A19V), DM84 (DM31+V64I), and DM85 (DM31+A73V). The results were obtained using GROMACS 2022.1.

FIG. 14 Average RMSF (Root Mean Square Fluctuations) plots of variants related to position C41. DM91 (DM31+C41V), DM150 (DM31+C41A), DM151 (DM31+C41S), DM143 (DM31+C41V+T12V), DM152 (DM31+T12V), and DM46 (DM31+C41T). The results were obtained using GROMACS 2022.1.

FIG. 15 average RMSF (Root Mean Square Fluctuations) of DM31, MNEI, DM70 (R84L), DM420 (R84Y) and DM424 (R84I). Each variant is depicted by a different color. DM70 and DM424 are expected to be as stable as DM31. The results were obtained using GROMACS 2022.1.

FIG. 16 Average RMSF (Root Mean Square Fluctuations) plots of MNEI, DM31, DM72 (DM31+N35T). The results were obtained using GROMACS 2022.1.

FIG. 17 Average RMSF (Root Mean Square Fluctuations) plots of MNEI, DM31, DM75 (DM31+E4Q). The results were obtained using GROMACS 2022.1.

FIG. 18 Average RMSF (Root Mean Square Fluctuations) plots of MNEI, DM31, DM57 (DM31+T33R). The results were obtained using GROMACS 2022.1.

FIG. 19 is a spider graph showing that savarina with 50% sugar reduction combined with Amai sweet protein is as sweet as a full sugar product.

FIG. 20 is a spider graph showing that the formulation of Halva spread with 80% sugar reduction and DM31 is sweeter than Halva spread with 80% sugar reduction without DM31.

FIGS. 21A and 21B VoroMQA analysis for packing and molecular dynamics for DM42 (I26T), DM65 (Q28K) and DM92 (I26T+Q28K) highlighting variants with improved packing which are thus expected to be more stable. (A) The calculated score for packing is represented by colors on the residue, where red represents a higher degree of packing (better) and blue represents a lower degree of packing (worse). The green colors in the end of the alpha-helix shows that the packing of DM42 and DM92 are improved in comparison to DM31 (for which this region is dark blue), packing of lysine and serine at position 28 is better than the wild-type glutamine, and packing of threonine at position 26 is better than the wild-type isoleucine. (maybe replace with a graph?) (B) Molecular dynamics average RMSF of DM31, MNEI, DM65 (Q28K), DM42 (126T) and DM92 (I26T+Q28K). Lower values represent less fluctuations. Each variant is depicted by a different color. DM92 is expected to be more stable than DM31, DM42 and DM65 due to the reduced RMSF values. The results were obtained using GROMACS 2022.1.

FIG. 22 Average RMSF (Root Mean Square Fluctuations) of DM31, MNEI, DM87 (D68N), DM43 (K25R), DM464 (K25R+D68N),). Each variant is depicted by a different color. When comparing the RMSF of the helix end region (25-30aa) of the different DM to DM31 (black line), DM43, DM464 demonstrate lower RMSF.

FIG. 23 VoroMQA analysis of packing. The calculated score for packing is depicted by the color of the residue, where red represents a higher degree of packing (better) and blue represents a lower degree of packing (worse). Note the change of colors in the sidechain of residue 28 showing that Substitution of Q28 to K increases local packing.

FIG. 24 Electrostatic potential surface (using APBS—Adaptive Poisson-Boltzmann Solver) of DM31, DM57 (T33R). Position 33 is marked by a red circle. DM57 is sweeter than DM31. Substitution of position 33 to arginine increases the positive charge in this region.

FIG. 25 Average RMSF (Root Mean Square Fluctuations) of DM31, MNEI, DM87 (D68N), DM108 (D68T) and DM61 (D68S). DM61, DM108 are expected to be more stable than DM31 in the region of Loop A2 (residues 26-35). Each variant is depicted by a different color. Substitution of D68S and D68T are expected to be more stable than substitution to D68N. The results were obtained using GROMACS 2022.1.

FIG. 26 Electrostatic potential surface (using APBS) of DM61 (D68S), DM87 (D68N), DM31, M108 (D68T). Position 68 is marked by a red circle. DM87 is sweeter than DM31. DM61 and DM108 are expected to be as sweet as DM87.

FIG. 27 Average RMSF (Root Mean Square Fluctuations) of DM31, MNEI, DM46 (C41T), DM150 (C41A) and DM151 (C41S). DM46 is expected to be more stable than DM31. The results were obtained using GROMACS 2022.1.

FIG. 28 Average RMSF (Root Mean Square Fluctuations) of DM31, MNEI, DM508 (V20I+Q28K), DM509 (V20I+K25R), DM96 (V20I), DM117 (V20I+V64I), DM43 (K25R), DM84 (V64I), DM65 (Q28K). All DMs are predicted to be as stable as DM31. The results were obtained using GROMACS 2022.1.

FIG. 29 Average RMSF (Root Mean Square Fluctuations) of DM31, MNEI, DM498 (C41SV64I+A73F+175L+F89V), DM77 (A19V), DM491. (V20I+C41A+A73F+175L+F89M). All DMs are expected to be at least as stable as DM31. The results were obtained using GROMACS 2022.1.

FIG. 30 is a spider graph showing that vinaigrette with 50% sugar reduction combined with Amai sweet protein is sweeter, compared to the vinaigrette with 50% sugar reduction and no-protein addition.

FIG. 31 Average RMSF (Root Mean Square Fluctuations) of DM31, MNEI, DM508 (V20I+Q28K), DM65 (Q28K) and DM96 (V20I). Each variant is depicted by a different color. DM508 is expected to be more stable in the Loop A2(26-30aa). The results were obtained using GROMACS 2022.1.

FIG. 32 Electrostatics potential of DM31, DM508 (V20I+Q28K) compared to DM96 (V20I) (containing Q28) and DM65 (Q28K). Position 28 is marked by a red circle. DM508 is expected to be as sweet as DM65.

FIG. 33 Electrostatic potential surface (using APBS) of DM87 (D68N), DM506 (V20I+D68N). Position 68 is marked by a red circle. DM87 is sweeter than DM31. DM506 is expected to be as sweet as DM87. Additional FIG. 1:

FIG. 34 Average RMSF of DM31, MNEI, DM65 (Q28K), DM69 (K25R+Q28K) and DM43 (K25R). Each variant is depicted by a different color. DM69 has overall lower fluctuations along the protein sequence, and specifically in the helix cap region. DM43 has less fluctuations in the helix cap region. The results were obtained using GROMACS 2022.1.

FIG. 35 Electrostatic potential surface (using APBS) of DM31, DM65 (Q28K) and DM69 (K25R+Q28K). Q28K is marked by a red circle. DM69 and DM65 are expected to be sweeter than DM31, in light of the electrostatics changes presented.

FIG. 36 Average RMSF (Root Mean Square Fluctuations) of DM31, MNEI, DM65 (Q28K), DM330 (Q28S) and DM66 (Q28R). Each variant is depicted by a different color. Arg and Ser at position 28 may be equivalent to Lys. The results were obtained using GROMACS 2022.1.

FIG. 37 Electrostatic potential surface (using APBS) of DM31, DM65 (Q28K), DM330 (Q28S) and DM66 (Q28R). Position 28 is marked by a red circle. All of these DMs are expected to be sweeter than DM31, based on electrostatics analysis.

FIG. 38 Sweetness intensity of DM65 (Q28K) relative to the sweetness of its precursor (DM31). A sensory panel had tasted both proteins and ranked their sweetness.

FIG. 38 shows the relative sweetness of DM65 and DM66 compared to DM31 in 10 Brix. Both proteins show increased sweetness by 15% and 19%, respectively, according to a sensory panel.

FIG. 39 Average RMSF (Root Mean Square Fluctuations) of DM31, MNEI, DM452 (C41A+V64L+I75V), DM432 (G16A+C41A+V64L+I75V), DM84 (V64I), DM103 (T75L) and DM150 (C41A). Each variant is depicted by a different color. DM452 is expected to be more stable in the Loops. The results were obtained using GROMACS 2022.1.

FIG. 40 Average RMSF (Root Mean Square Fluctuations) of DM31, MNEI, DM506 (D68N+V20I), DM87 (D68N), and DM96 (V20I). Each variant is depicted by a different color. DM452 is expected to be more stable in the Loops. The results were obtained using GROMACS 2022.1.

FIG. 41 shows APBS analysis.

FIG. 42 is a spider graph showing that teriyaki sauce with 70% sugar reduction combined with Amai sweet protein is sweeter, compared to Teriyaki sauce with 70% sugar reduction and no-protein addition.

FIG. 43 is a spider graph showing that in chocolate peanut butter cups, the peanut butter spread with 75% sugar reduction combined with Amai sweet protein is sweeter, compared to the peanut butter spread with 75% sugar reduction and no-protein addition.

FIG. 44 is a spider graph showing that sweet chili sauce with 50% sugar reduction combined with Amai sweet protein is as sweet as a full sugar product.

FIG. 45 is a spider graph showing that Thousand island sauce with 62% sugar reduction combined with Amai sweet protein is sweeter, compared to thousand island sauce with 62% sugar reduction and no-protein addition.

FIG. 46 is a spider graph showing that Dark Chocolate with 70% sugar reduction and sweet protein DM-31 is sweeter than Dark Chocolate with 70% sugar reduction.

FIG. 47 are histograms showing that the sweetness intensity of DM31 in rice flour with 40% moisture content is stable after heat treatment at 60° C. and 70° C. for 18 hours in open plates and reconstitution with water.

### DETAILED DESCRIPTION OF EMBODIMENTS

Artificial low-calorie sweeteners are readily available in the market, yet many have significant side effects. For example, saccharin, widely used to sweeten foods and beverages without added calories or carbohydrates, has been linked to cancers such as bladder cancer. Thus, there is a significant need for replacements of the currently available artificial low-calorie sweeteners that will provide both an optimal sensory profile and be suitable for use in food products and beverages.

The present disclosure relates to novel proteins that on one hand were shown to be sweet and on the other hand were shown to be stable. As described herein, the novel proteins were identified by various computational methods.

Surprisingly, the inventors have found that introducing various specific modifications in an amino acid sequence of a reference protein, including, inter alia, deletions or substitutions of amino acids, resulted in a protein having at least one improved food-related property.

Specifically, as shown in the Examples below, the new proteins (denoted herein as “modified protein” or “designer protein”) exhibited an improved sensory profile and/or thermal stability and/or reduced hydrophobicity as compared to the reference protein. The sensory profile, as described herein, relates to a taste profile of the modified protein (e.g., sweetness potency).

Based on thee results, it was suggested that the new proteins can be used in food and beverage applications for preparation of a variety of edible products.

Thus, in its broadest aspect, the present disclosure relates to a modified protein comprising an amino acid sequence that has at least one amino acid deletion, replacement (substitution) and/or insertion as compared with an amino acid sequence of a reference protein. As described herein, the modified protein has at least one improved food-related property as compared with the reference protein.

In the present disclosure, the modified protein also denoted at times as a designer protein may be considered as a variant of a reference protein. The term “variant” as used herein refers to a sequence that contains at least one amino acid modification as compared to the reference protein. The present disclosure also encompasses a modified nucleic acid sequence, including corresponding substitution, deletion or insertion of codons similar to the ones in the modified protein.

The present disclosure is not limited to specific number of amino acid modifications made in the reference protein that eventually results in the formation of the modified protein.

A modification as used herein refers to a modification in one or more amino acids of the reference protein and encompasses amino acid substitution (replacement), amino acid deletion, amino acid insertion or any combination thereof.

In some examples, a modification may be an amino acid insertion.

In some embodiments, the modified protein comprises an amino acid sequence having at least two, at least three, at least four, at least five amino acid insertions as compared to the reference protein.

In some examples, a modification may be an amino acid deletion.

In some embodiments, the modified protein comprises an amino acid sequence having at least two, at least three, at least four, at least five amino acid deletions as compared to the reference protein.

In some examples, a modification may be an amino acid substitution.

In some embodiments, the modified protein comprises an amino acid sequence having at least two, at least three, at least four, at least five amino acid substitutions as compared to the reference protein.

As described herein, the modified protein may result from amino acid modifications (substitutions or deletions) at various regions of the protein. “Regions of the protein” as used herein, refers to an amino acid sequence or structural motif that is part of the protein sequence (amino acid sequence) or structure. Non-limiting examples of protein regions include protein surface, protein core, protein loop, secondary structure elements, secondary structure capping, disulfide, binding-site, linker, hydrophobic-patch, or protein hydrophobic region.

The amino acid modifications in the reference protein is not limited to a specific protein region or sequence. Regions of the reference protein that may include the amino acid modifications include the reference protein surface, hydrophobic core, or regions called loop regions (also denoted as regions lacking secondary structures), edges of secondary structures (also denoted secondary structure capping regions), disulfide regions, binding-site regions, linker regions, and hydrophobic-patch regions.

As used herein a “reference surface region,” “reference core region,” or “reference disulfide bond or loop region,” may refer to the corresponding region of the reference protein.

In some embodiments, the reference protein may be modified (e.g. substituted) within a confined region within the reference protein structure and/or sequence. In some embodiments, the reference protein may be modified (e.g. substituted) in the surface region. In some embodiments, the reference protein may be modified (e.g. substituted) in the core region. In some embodiments, the reference protein may be modified (e.g. substituted) by disulfide bonds. In some embodiments, the reference protein may be modified (e.g. substituted) in loop regions. In some embodiments, the amino acid modifications (replacements) are located on the surface of the reference protein.

In some embodiments, the reference protein may be substituted with a confined region that is not in the area adjacent to the predicted or known binding site of the reference protein to the receptor. In this context, ‘adjacent’ may mean 4-7 Å from the binding interface.

In some embodiments, the reference protein may be substituted at different regions within the reference protein structure and/or sequence. In some embodiments, the reference protein may be substituted at least in the surface region, the core region, the disulfide bond or loop regions, or any combination thereof.

As used herein, the protein surface region is the area with partial or full solvent accessibility (SASA—solvent accessible surface area). The protein core region as used herein, is the area not accessible to solvents with an amino-acid relative SASA (solvent accessible surface area) of less than 50% or, for the inner core, less than 20%.

As shown in the Examples below, the amino acid sequence of the reference protein may be modified in at least one of (i) an alpha helix of the reference protein, (ii) a core of the reference protein, (iii) a sweet loop of the reference protein, (iv) a linker region of the reference protein or (v) any combination thereof.

The amino acid sequence of the reference protein may be modified such that the modification affects core packing and/or electrostatics of the reference protein, the modified protein or both.

The amino acid sequence of the reference protein may be modified in a linker region of the reference protein.

The amino acid sequence of the reference protein may be modified in a linker region of the reference protein such that that at least one amino acid is deleted.

The modified protein comprises at least one, at times at least two, at times at least three amino acid deletions in the reference protein.

Hence, in accordance with some aspects, the present disclosure provides a protein comprising an amino acid sequence that comprise at least one amino acid modification in in at least one of (i) an alpha helix of the reference protein, (ii) a core of the reference protein, (iii) a sweet loop of the reference protein, (iv) a linker region of the reference protein or (v) any combination thereof, wherein the reference protein has an amino acid sequence selected from the group consisting of SEQ ID NO:8, SEQ ID NO:9 and SEQ ID NO:10.

In the following text, when referring to the modified protein it is to be understood as also referring to the food products disclosed herein. Thus, whenever providing a feature with reference to the modified protein, it is to be understood as defining the same feature with respect to the food product mutatis mutandis.

As shown in Example 1 below, molecular dynamics tools were used for designing new modified proteins based on optimization of various regions of the reference protein. Specifically, using Rosetta Energy Unit (REU) scores for the newly designed modified proteins, it was possible to predict those proteins that would exhibit improved properties, specifically at least one improved food-related property as described herein.

Energetic calculations can be applied to the entire amino acid sequence or, alternatively, be restricted to specific regions or selected amino acids within the protein. In the latter (different regions or selected amino acids), the information may be integrated to measure the entire protein.

Calculation of each one of the amino acid sequences (e.g., a modified protein) may be done by combining physico-based (also known as biophysical methods) and statistics-based potentials (also known as knowledge-based potentials or informatics methods), such as by using the Rosetta Energy Unit (REU). Rosetta Energy Unit (REU) is an algorithm of the Rosetta software, a package of algorithms for computational modeling and protein structures analysis. The Rosetta software enables notable scientific advances in computational biology, including de novo protein design, enzyme design, ligand docking, and structure prediction of biological macromolecules and macromolecular complexes. Rosetta energy function is a combination of physical and statistical based potentials that does not match with any actual physical energy units. Rosetta energies are on an arbitrary scale and sometimes referred to as REU (for “Rosetta Energy Unit”).

In some embodiments, the REU may be calculated for the entire protein sequence comprising the at least one amino acid modification. In some other embodiments, the REU may be calculated for at least one region comprising the at least one amino acid modification of the entire protein sequence. In some other embodiments, the REU may be calculated for at least one amino acid modification in the entire protein sequence.

In some embodiments, the modified protein has an energy lower than about −315, at times lower than about −317, at times lower than about −319, at times lower than about −321, at times lower than about −322, at times lower than about −324 given in REU.

In some embodiments, the modified protein has an energy of about −315 given in REU. In some embodiments, the modified protein has an energy of about −317 given in REU. In some embodiments, the modified protein has an energy of about −319 given in REU. In some embodiments, the modified protein has an energy of about −321 given in REU. In some embodiments, the modified protein has an energy of about −324 given in REU. In some embodiments, the modified protein has an energy of about −326 given in REU.

In some embodiments, the modified protein comprises an amino acid sequence 40% to 98% identical to an amino acid sequence of the reference protein. In some embodiments, the modified protein comprises an amino acid sequence 90% to 98% identical to the reference amino acid sequence.

In some embodiments, the modified protein comprises an amino acid sequence 60% to 90% identical to the reference amino acid sequence. In some embodiments, the modified protein comprises an amino acid sequence 70% to 90% identical to the reference amino acid sequence.

In some embodiments, the modified protein comprises an amino acid sequence having at least 60%, at least 65%, at least 70%, at least 75%, at least 80%, at least 85%, at least 90%, at least 95%, or at least 98% identity to the reference amino acid sequence.

In some embodiments, the modified protein comprises an amino acid sequence having 90% to 98% identity to the reference amino acid sequence.

The % identity between two or more amino acid sequences is determined when the two or more sequences are compared and aligned for maximum correspondence. In the context of the present disclosure, sequences (amino acid) as described herein having % identity are considered to have the same function/activity as the reference sequence to which identity is calculated.

In some embodiments, the modified protein comprises an amino acid sequence 40% to 98%, similar to an amino acid sequence of the reference protein. In some embodiments, the modified protein comprises an amino acid sequence 90% to 98% similarity to the reference amino acid sequence.

In some embodiments, the modified protein comprises an amino acid sequence 60% to 90% similarity to the reference amino acid sequence. In some embodiments, the modified protein comprises an amino acid sequence 70% to 90% similar to the reference amino acid sequence.

In some embodiments, the modified protein comprises an amino acid sequence having at least 60%, at least 65%, at least 70%, at least 75%, at least 80%, at least 85%, at least 90%, at least 95%, or at least 98% similarity to the reference amino acid sequence.

In some embodiments, the modified protein comprises an amino acid sequence having 90% to 98% similarity to the reference amino acid sequence.

In some embodiments, the reference protein is denoted by SEQ ID NO:8 also denoted as MNEI. SEQ ID NO:8 as refers herein having the following amino acid sequence:

GEWEIIDIGPFTQNLGKFAVDEENKIGQYGRLTFNKVIRPCMKKTIYENE
GFREIKGYEYQLYVYASDKLFRADISEDYKTRGRKLLRFNGPVPPP

As appreciated, MNEI is a synthetic protein made of a combination of chain A Monellin (GenBank Entry No. P02881) and chain B Monellin (GenBank Entry No. P02882).

The main difference between wild-type Monellin and MNEI is a region the two Monellin subunits are connected into a single-chain Monellin termed MNEI. The amino acid sequence between amino acid T46 and amino acid 156 is denoted as a loop region, connects the two Monellin subunits.

The modified protein refers in some examples to a variant of MNEI and in some further examples to a variant of MNEI having modification in the linker (loop) region.

As shown in the examples below, modification in the loop region of the modified protein increased the sweetness and/or stability of the reference protein. specifically, deletion of amino acids residues in the protein loop and beta strand edges results in a modified protein that is stable as compared to the reference protein, being in some examples, MNEI.

Hence, in accordance with some aspects, the present disclosure provides a protein comprising an amino acid sequence that has amino acid deletions in at least three amino acids located between amino acid T46 and amino acid 156, as compared to a reference protein, wherein the reference protein has an amino acid sequence selected from the group consisting of SEQ ID NO:8, SEQ ID NO:9 and SEQ ID NO:10.

In accordance with some other aspects, the present disclosure provides a provides a modified protein comprising an amino acid sequence that has amino acid deletions in at least three amino acids located between amino acid T46 and amino acid 156, as compared to a reference protein, wherein the reference protein has an amino acid sequence selected from the group consisting of SEQ ID NO:8, SEQ ID NO:9 and SEQ ID NO:10 and wherein the modified protein has at least one improved food-related property as compared to the reference protein.

In accordance with the present disclosure, the reference protein is a synthetic protein. When referring to a synthetic protein it should be understood as protein that was not found in nature and is thus considered as a synthetic protein.

As noted above, in some examples, the reference protein is MNEI denoted herein by SEQ ID NO:8.

Hence, in accordance with some aspects, the present disclosure provides a modified protein comprising an amino acid sequence that has amino acid deletions in at least three amino acids located between amino acid T46 and amino acid 156, as compared to a reference protein, and wherein the reference protein has an amino acid sequence as set forth in SEQ ID NO:8.

As shown in the Examples below that form part of this patent application, deletion of three amino acids E50, F52 and R53 from the amino acid sequence of MNEI (DM29 denoted herein as SEQ ID NO:11) suggested that the modified protein would have increased stability based on REU value of −319.65 as compared to the REU value of MNEI of −315.35.

Hence, in accordance with some aspects, the present disclosure provides a modified protein comprising an amino acid sequence that has amino acid deletions in at least amino acid E50, F52 and R53 as compared to a reference protein, and wherein the reference protein has an amino acid sequence set forth in SEQ ID NO:8.

In accordance with some embodiments, the modified protein comprises amino acid substitutions as compared to the reference protein.

In some embodiments, the modified protein comprises an amino acid sequence having at least two, at least three, at least four, at least five amino acid substitutions as compared to the reference protein.

In some embodiments, the modified protein comprises an amino acid sequence having at least five amino acid substitutions, at least six, at least seven, at least eight, at least nine amino acid substitutions as compared to the reference protein.

In some embodiments, the modified protein comprises between five and twenty amino acid substitutions, as compared to a reference protein (reference amino acid sequence) at times between five and ten amino acid substitutions.

In accordance with some other embodiments, the modified protein comprises at least five amino acid substitutions as compared to a reference protein, wherein the reference protein has an amino acid sequence set forth in SEQ ID NO:8.

Hence, in accordance with some aspects, the present disclosure provides a modified protein comprising an amino acid sequence that has amino acid deletions in at least three amino acids located between amino acid T46 and amino acid 156 and amino acid substitutions in at least five amino acids as compared to a reference protein, and wherein the reference protein has an amino acid sequence set forth in SEQ ID NO:8.

In accordance with some other aspects, the present disclosure provides a modified protein comprising an amino acid sequence that has amino acid deletions in amino acid E50, F52 and R53 as compared to a reference protein and amino acid substitutions in at least five amino acids as compared to a reference protein, and wherein the reference protein has an amino acid sequence set forth in SEQ ID NO:8.

In some embodiments, the at least five amino acids substitutions comprise substitutions in amino acids E2, E23 and Y65 as compared to the reference protein.

In accordance with some other aspects, the present disclosure provides a modified protein comprising an amino acid sequence that has amino acid deletions in at least three amino acids located between amino acid T46 and amino acid 156 as compared to a reference protein and amino acid substitutions in at least five amino acids as compared to a reference protein, wherein the at least five amino acids substitutions comprise substitutions in amino acids E2, E23 and Y65 as compared to the reference protein and wherein the reference protein has an amino acid sequence set forth in SEQ ID NO:8.

In accordance with some other aspects, the present disclosure provides a modified protein comprising an amino acid sequence that has amino acid deletions in amino acid E50, F52 and R53 as compared to a reference protein and amino acid substitutions in at least five amino acids as compared to a reference protein, wherein the at least five amino acids substitutions comprise substitutions in amino acids E2, E23 and Y65 as compared to the reference protein and wherein the reference protein has an amino acid sequence set forth in SEQ ID NO:8.

In some embodiments, the at least five amino acids substitutions comprise a substitution in amino acids L70 as compared to the reference protein.

In accordance with some other aspects, the present disclosure provides a modified protein comprising an amino acid sequence that has amino acid deletions in at least three amino acids located between amino acid T46 and amino acid 156, as compared to a reference protein, and amino acid substitutions in at least five amino acids as compared to a reference protein, wherein the at least five amino acids substitutions comprise a substitution in amino acid L70 as compared to the reference protein and wherein the reference protein has an amino acid sequence set forth in SEQ ID NO:8.

In accordance with some other aspects, the present disclosure provides a modified protein comprising an amino acid sequence that has amino acid deletions in amino acid E50, F52 and R53 as compared to a reference protein and amino acid substitutions in at least five amino acids as compared to a reference protein, wherein the at least five amino acids substitutions comprise a substitution in amino acid L70 as compared to the reference protein and wherein the reference protein has an amino acid sequence set forth in SEQ ID NO:8.

In accordance with some other aspects, the present disclosure provides a modified protein comprising an amino acid sequence that has amino acid deletions in at least three amino acids located between amino acid T46 and amino acid 156, as compared to a reference protein, and amino acid substitutions in at least five amino acids as compared to a reference protein, wherein the at least five amino acids substitutions comprise substitutions in amino acids E2, E23, Y65 and L70 as compared to the reference protein and wherein the reference protein has an amino acid sequence set forth in SEQ ID NO:8.

In accordance with some other aspects, the present disclosure provides a modified protein comprising an amino acid sequence that has amino acid deletions in amino acid E50, F52 and R53 as compared to a reference protein and amino acid substitutions in at least five amino acids as compared to a reference protein, wherein the at least five amino acids substitutions comprise substitutions in amino acids E2, E23, Y65 and L70 as compared to the reference protein and wherein the reference protein has an amino acid sequence set forth in SEQ ID NO:8.

As appreciated, the amino acid substitutions are not limited to specific region of the reference protein and may be located in the entire reference protein structure/sequence as described herein.

In some embodiments, the modified protein comprises an amino acid substitution in at least one amino acid selected from the group consisting of E4, T12, A19, V20, K25, 126, Q28, R31, T33, N35, C41, Q61, V64, D68, A73, 175, R84 and F89 as compared to said reference protein.

In accordance with some other aspects, the present disclosure provides a modified protein comprising an amino acid sequence that has amino acid deletions in at least three amino acids located between amino acid T46 and amino acid 156, as compared to a reference protein, and amino acid substitutions in at least five amino acids as compared to a reference protein, wherein the at least five amino acids substitutions comprise substitutions in amino acids E2, E23, Y65 and L70 and in at least one amino acid selected from the group consisting of E4, T12, A19, V20, K25, 126, Q28, R31, T33, N35, C41, Q61, V64, D68, A73, 175, R84 and F89 as compared to the reference protein and wherein the reference protein has an amino acid sequence set forth in SEQ ID NO:8.

In accordance with some other aspects, the present disclosure provides a modified protein comprising an amino acid sequence that has amino acid deletions in amino acid E50, F52 and R53 as compared to a reference protein and amino acid substitutions in at least five amino acids as compared to a reference protein, wherein the at least five amino acids substitutions comprise substitutions in amino acids E2, E23, Y65 and L70 and in at least one amino acid selected from the group consisting of E4, T12, A19, V20, K25, 126, Q28, R31, T33, N35, C41, Q61, V64, D68, A73, 175, R84 and F89 as compared to the reference protein and wherein the reference protein has an amino acid sequence set forth in SEQ ID NO:8.

In some embodiments, the modified protein comprises an amino acid substitution in at least one amino acid selected from the group consisting of Q28, C41, and D68 as compared to said reference protein.

In accordance with some other aspects, the present disclosure provides a modified protein comprising an amino acid sequence that has amino acid deletions in at least three amino acids located between amino acid T46 and amino acid 156, as compared to a reference protein, and amino acid substitutions in at least five amino acids as compared to a reference protein, wherein the at least five amino acids substitutions comprise substitutions in amino acids E2, E23, Y65 and L70 and in at least one amino acid selected from the group consisting of Q28, C41, and D68 as compared to the reference protein and wherein the reference protein has an amino acid sequence set forth in SEQ ID NO:8.

In accordance with some other aspects, the present disclosure provides a modified protein comprising an amino acid sequence that has amino acid deletions in amino acid E50, F52 and R53 as compared to a reference protein and amino acid substitutions in at least five amino acids as compared to a reference protein, wherein the at least five amino acids substitutions comprise substitutions in amino acids E2, E23, Y65 and L70 and in at least one amino acid selected from the group consisting of Q28, C41, and F68 as compared to the reference protein and wherein the reference protein has an amino acid sequence set forth in SEQ ID NO:8.

In some embodiments, the modified protein comprises an amino acid substitution in at least one amino acid selected from the group consisting of A19, V20, K25, 126, T33, N35 and R84 as compared to said reference protein.

In accordance with some other aspects, the present disclosure provides a modified protein comprising an amino acid sequence that has amino acid deletions in at least three amino acids located between amino acid T46 and amino acid 156, as compared to a reference protein, and amino acid substitutions in at least five amino acids as compared to a reference protein, wherein the at least five amino acids substitutions comprise substitutions in amino acids E2, E23, Y65 and L70 and in at least one amino acid selected from the group consisting of A19, V20, K25, 126, Q28, T33, and N35 as compared to the reference protein and wherein the reference protein has an amino acid sequence set forth in SEQ ID NO:8.

In accordance with some other aspects, the present disclosure provides a modified protein comprising an amino acid sequence that has amino acid deletions in amino acid E50, F52 and R53 as compared to a reference protein and amino acid substitutions in at least five amino acids as compared to a reference protein, wherein the at least five amino acids substitutions comprise substitutions in amino acids E2, E23, Y65 and L70 and in at least one amino acid selected from the group consisting of A19, V20, K25, 126, Q28, T33, and N35 as compared to the reference protein and wherein the reference protein has an amino acid sequence set forth in SEQ ID NO:8.

In some embodiments, the modified protein comprises an amino acid substitution in at least one amino acid selected from the group consisting of E4, T12, R31, V64 and A73 as compared to said reference protein.

In accordance with some other aspects, the present disclosure provides a modified protein comprising an amino acid sequence that has amino acid deletions in at least three amino acids located between amino acid T46 and amino acid 156, as compared to a reference protein, and amino acid substitutions in at least five amino acids as compared to a reference protein, wherein the at least five amino acids substitutions comprise substitutions in amino acids E2, E23, Y65 and L70 and in at least one amino acid selected from the group consisting of E4, T12, R31, V64, A73, and R84 as compared to the reference protein and wherein the reference protein has an amino acid sequence set forth in SEQ ID NO:8.

In accordance with some other aspects, the present disclosure provides a modified protein comprising an amino acid sequence that has amino acid deletions in amino acid E50, F52 and R53 as compared to a reference protein and amino acid substitutions in at least five amino acids as compared to a reference protein, wherein the at least five amino acids substitutions comprise substitutions in amino acids E2, E23, Y65 and L70 and in at least one amino acid selected from the group consisting of E4, T12, R31, V64, A73, and R84 as compared to the reference protein and wherein the reference protein has an amino acid sequence set forth in SEQ ID NO:8.

In some embodiments, the modified protein comprises an amino acid substitution in at least one amino acid selected from the group consisting of A19, V20, K25, 126, Q28, T33, C41 and D68 as compared to said reference protein.

In accordance with some other aspects, the present disclosure provides a modified protein comprising an amino acid sequence that has amino acid deletions in at least three amino acids located between amino acid T46 and amino acid 156, as compared to a reference protein, and amino acid substitutions in at least five amino acids as compared to a reference protein, wherein the at least five amino acids substitutions comprise substitutions in amino acids E2, E23, Y65 and L70 and in at least one amino acid selected from the group consisting of A19, V20, K25, 126, Q28, T33, C41 and D68 as compared to the reference protein and wherein the reference protein has an amino acid sequence set forth in SEQ ID NO:8.

In accordance with some other aspects, the present disclosure provides a modified protein comprising an amino acid sequence that has amino acid deletions in amino acid E50, F52 and R53 as compared to a reference protein and amino acid substitutions in at least five amino acids as compared to a reference protein, wherein the at least five amino acids substitutions comprise substitutions in amino acids E2, E23, Y65 and L70 and in at least one amino acid selected from the group consisting of A19, V20, K25, 126, Q28, T33, C41 and D68 as compared to the reference protein and wherein the reference protein has an amino acid sequence set forth in SEQ ID NO:8.

In some embodiments, the modified protein comprises an amino acid substitution in at least one amino acid selected from the group consisting of A19, V20, K25, 126, Q28, T33, C41 and D68 as compared to said reference protein.

In accordance with some other aspects, the present disclosure provides a modified protein comprising an amino acid sequence that has amino acid deletions in at least three amino acids located between amino acid T46 and amino acid 156, as compared to a reference protein, and amino acid substitutions in at least five amino acids as compared to a reference protein, wherein the at least five amino acids substitutions comprise substitutions in amino acids E2, E23, Y65 and L70 and in at least one amino acid selected from the group consisting of A19, V20, K25, 126, Q28, T33, C41 and D68 as compared to the reference protein and wherein the reference protein has an amino acid sequence set forth in SEQ ID NO:8.

In accordance with some other aspects, the present disclosure provides a modified protein comprising an amino acid sequence that has amino acid deletions in amino acid E50, F52 and R53 as compared to a reference protein and amino acid substitutions in at least five amino acids as compared to a reference protein, wherein the at least five amino acids substitutions comprise substitutions in amino acids E2, E23, Y65 and L70 and in at least one amino acid selected from the group consisting of A19, V20, K25, 126, Q28, T33, C41 and D68 as compared to the reference protein and wherein the reference protein has an amino acid sequence set forth in SEQ ID NO:8.

As noted above, the modifications, including, inter alia, the amino acid substitutions are not limited to specific regions of the reference protein and in accordance with some examples, are made in regions predicted to improve the reference protein properties.

In some embodiments, the at least five amino acids substitutions comprise substitutions in at least one amino acid that (i) stabilizes alpha helix structure, (ii) reduce aggregation, (iii) affect core repacking, (iv) affect electrostatics, or (v) any combination thereof of the reference protein, the modified protein or both.

In some embodiments, the at least five amino acids substitutions comprise substitutions in at least one amino acid located in (i) alpha helix, (ii) core, (iii) sweet loop or (iv) any combination thereof of the reference protein.

In some embodiments, the at least five amino acids substitutions comprise substitutions in at least one amino acid that affect core repacking of the reference protein, the modified protein or both.

In some embodiments, the at least five amino acids substitutions comprise substitutions in at least one amino acid that are located in the core of the reference protein.

The core of the reference protein, for example, MNEI is partially (half) exposed to the surface and is prone to water interferences. It was suggested that there are holes that make the core accessible to surface water (the protein is small so the core may be half exposed). It was thus suggested that modifications in the core region affecting core repacking would decrease accessibility to water and hence stabilize the modified protein. Hence, the term modification in the core repacking as used herein refers to any modification that affect core repacking and thus decreased accessibility to water.

In some embodiments, the modified protein comprises an amino acid substitution in at least one amino acid residue selected from the group consisting of T12, C41, A19, V20, A73, 175, F89, G16, L32, V37, L62 and V64 as compared to the reference protein.

In some embodiments, the modified protein comprises an amino acid substitution in at least one amino acid residue selected from the group consisting of T12, C41, A19, V20, A73, 175 and F89 as compared to the reference protein.

In some embodiments, the modified protein comprises an amino acid substitution in at least one amino acid residue selected from the group consisting of A19 and V20 as compared to the reference protein.

In accordance with some other aspects, the present disclosure provides a modified protein comprising an amino acid sequence that has amino acid deletions in at least three amino acids located between amino acid T46 and amino acid 156, as compared to a reference protein, and amino acid substitutions in at least five amino acids as compared to a reference protein, wherein the at least five amino acids substitutions comprise substitutions in amino acids E2, E23, Y65 and L70 and in at least one amino acid selected from the group consisting of A19 and V20 as compared to the reference protein and wherein the reference protein has an amino acid sequence set forth in SEQ ID NO:8.

In accordance with some other aspects, the present disclosure provides a modified protein comprising an amino acid sequence that has amino acid deletions in amino acid E50, F52 and R53 as compared to a reference protein and amino acid substitutions in at least five amino acids as compared to a reference protein, wherein the at least five amino acids substitutions comprise substitutions in amino acids E2, E23, Y65 and L70 and in at least one amino acid selected from the group consisting of A19 and V20 as compared to the reference protein and wherein the reference protein has an amino acid sequence set forth in SEQ ID NO:8.

In some embodiments, the at least five amino acids substitutions comprise substitutions in at least one amino acid that stabilizes alpha helix structure of the reference protein, the modified protein or both.

The term stabilizes alpha helix structure as used herein refers to any modification that affect the alpha helix structure of the reference protein, the modified protein or both. Such modifications include modifications in any one of amino acids residues K25, 126, Q28 of the reference protein.

In some embodiments, the modified protein comprises a substitution in at least one amino acid K25, 126, Q28 or any combination thereof as compared to the reference protein.

In accordance with some other aspects, the present disclosure provides a modified protein comprising an amino acid sequence that has amino acid deletions in at least three amino acids located between amino acid T46 and amino acid 156, as compared to a reference protein, and amino acid substitutions in at least five amino acids as compared to a reference protein, wherein the at least five amino acids substitutions comprise substitutions in amino acids E2, E23, Y65 and L70 and in at least one amino acid selected from the group consisting of K25, 126 and Q28 as compared to the reference protein and wherein the reference protein has an amino acid sequence set forth in SEQ ID NO:8.

In accordance with some other aspects, the present disclosure provides a modified protein comprising an amino acid sequence that has amino acid deletions in amino acid E50, F52 and R53 as compared to a reference protein and amino acid substitutions in at least five amino acids as compared to a reference protein, wherein the at least five amino acids substitutions comprise substitutions in amino acids E2, E23, Y65 and L70 and in at least one amino acid selected from the group consisting of K25, 126 and Q28 as compared to the reference protein and wherein the reference protein has an amino acid sequence set forth in SEQ ID NO:8.

In some embodiments, the at least five amino acids substitutions comprise substitutions in at least one amino acid that is located in the sweet loop of the reference protein.

The sweet loop as used herein refers to a region in the reference protein between residues 63 and 68.

In some embodiments, the modified protein comprises a substitution in at least one amino acid selected from the group consisting of T33 and D68 as compared to the reference protein.

In accordance with some other aspects, the present disclosure provides a modified protein comprising an amino acid sequence that has amino acid deletions in at least three amino acids located between amino acid T46 and amino acid 156, as compared to a reference protein, and amino acid substitutions in at least five amino acids as compared to a reference protein, wherein the at least five amino acids substitutions comprise substitutions in amino acids E2, E23, Y65 and L70 and in at least one amino acid selected from the group consisting of T33 and D68 as compared to the reference protein and wherein the reference protein has an amino acid sequence set forth in SEQ ID NO:8.

In accordance with some other aspects, the present disclosure provides a modified protein comprising an amino acid sequence that has amino acid deletions in amino acid E50, F52 and R53 as compared to a reference protein and amino acid substitutions in at least five amino acids as compared to a reference protein, wherein the at least five amino acids substitutions comprise substitutions in amino acids E2, E23, Y65 and L70 and in at least one amino acid selected from the group consisting of T33 and D68 as compared to the reference protein and wherein the reference protein has an amino acid sequence set forth in SEQ ID NO:8.

In some embodiments, the at least five amino acids substitutions comprise substitutions in at least one amino acid that reduces aggregation of the reference protein, the modified protein or both.

The term reduce protein aggregation as used herein refers to any modification that affect protein aggregation Such modification include modification in amino acid residue C41.

In some embodiments, the modified protein comprises a substitution in amino acid C41 as compared to the reference protein.

Based on the computational analysis, it was suggested that amino acid C41 is prone to disulfide bond and promoting aggregation and hence it was suggested that any modification in this residue may reduce protein aggregation.

In accordance with some other aspects, the present disclosure provides a modified protein comprising an amino acid sequence that has amino acid deletions in at least three amino acids located between amino acid T46 and amino acid 156, as compared to a reference protein, and amino acid substitutions in at least five amino acids as compared to a reference protein, wherein the at least five amino acids substitutions comprise substitutions in amino acids E2, E23, Y65, L70 and C41 as compared to the reference protein and wherein the reference protein has an amino acid sequence set forth in SEQ ID NO:8.

In accordance with some other aspects, the present disclosure provides a modified protein comprising an amino acid sequence that has amino acid deletions in amino acid E50, F52 and R53 as compared to a reference protein and amino acid substitutions in at least five amino acids as compared to a reference protein, wherein the at least five amino acids substitutions comprise substitutions in amino acids E2, E23, Y65, L70 and C41 as compared to the reference protein and wherein the reference protein has an amino acid sequence set forth in SEQ ID NO:8.

In some embodiments, the at least five amino acids substitutions comprise substitutions in amino acids that affect electrostatics of the reference protein, the modified protein or both.

The term electrostatics as used herein refers to any modification that affect electrostatics Such modification include modification in at least one amino acid residue T33, E4 or any combinations thereof.

In some embodiments, the modified protein comprises a substitution in at least one amino acid residue T33, E4 or any combinations thereof as compared to the reference protein.

In accordance with some other aspects, the present disclosure provides a modified protein comprising an amino acid sequence that has amino acid deletions in at least three amino acids located between amino acid T46 and amino acid 156, as compared to a reference protein, and amino acid substitutions in at least five amino acids as compared to a reference protein, wherein the at least five amino acids substitutions comprise substitutions in amino acids E2, E23, Y65 and L70 and in at least one amino acid selected from the group consisting of T33 and E4 as compared to the reference protein and wherein the reference protein has an amino acid sequence set forth in SEQ ID NO:8.

In accordance with some other aspects, the present disclosure provides a modified protein comprising an amino acid sequence that has amino acid deletions in amino acid E50, F52 and R53 as compared to a reference protein and amino acid substitutions in at least five amino acids as compared to a reference protein, wherein the at least five amino acids substitutions comprise substitutions in amino acids E2, E23, Y65 and L70 and in at least one amino acid selected from the group consisting of T33 and E4 as compared to the reference protein and wherein the reference protein has an amino acid sequence set forth in SEQ ID NO:8.

In some embodiments, the modified protein comprises an amino acid substitution in at least amino acid E4 as compared to said reference protein. In some embodiments, the modified protein comprises an amino acid substitution in at least amino acid T12 as compared to said reference protein. In some embodiments, the modified protein comprises an amino acid substitution in at least amino acid A19 as compared to said reference protein. In some embodiments, the modified protein comprises an amino acid substitution in at least amino acid V20 as compared to said reference protein. In some embodiments, the modified protein comprises an amino acid substitution in at least amino acid K25 as compared to said reference protein. In some embodiments, the modified protein comprises an amino acid substitution in at least amino acid 126 as compared to said reference protein. In some embodiments, the modified protein comprises an amino acid substitution in at least amino acid Q28 as compared to said reference protein. In some embodiments, the modified protein comprises an amino acid substitution in at least amino acid R31 as compared to said reference protein. In some embodiments, the modified protein comprises an amino acid substitution in at least amino acid T33 as compared to said reference protein. In some embodiments, the modified protein comprises an amino acid substitution in at least amino acid N35 as compared to said reference protein. In some embodiments, the modified protein comprises an amino acid substitution in at least amino acid C41 as compared to said reference protein. In some embodiments, the modified protein comprises an amino acid substitution in at least amino acid Q61 as compared to said reference protein. In some embodiments, the modified protein comprises an amino acid substitution in at least amino acid V64 as compared to said reference protein. In some embodiments, the modified protein comprises an amino acid substitution in at least amino acid D68 as compared to said reference protein. In some embodiments, the modified protein comprises an amino acid substitution in at least amino acid A73 as compared to said reference protein. In some embodiments, the modified protein comprises an amino acid substitution in at least amino acid 175 as compared to said reference protein. In some embodiments, the modified protein comprises an amino acid substitution in at least amino acid R84 as compared to said reference protein. In some embodiments, the modified protein comprises an amino acid substitution in at least amino acid F89 as compared to said reference protein.

In some embodiments, the modified protein comprises an amino acid substitution in amino acid Q28 and in at least amino acid selected from the group consisting of V20, K25, 126 and R31 as compared to said reference protein. In some embodiments, the modified protein comprises an amino acid substitution in amino acid Q28 and V20 as compared to said reference protein. In some embodiments, the modified protein comprises an amino acid substitution in amino acid Q28 and K25, as compared to said reference protein. In some embodiments, the modified protein comprises an amino acid substitution in amino acid Q28 and 126 as compared to said reference protein. In some embodiments, the modified protein comprises an amino acid substitution in amino acid Q28 and R31 as compared to said reference protein.

In some embodiments, the modified protein comprises an amino acid substitution in amino acid D68 and in at least amino acid selected from the group consisting of V20, R31, R84 as compared to said reference protein. In some embodiments, the modified protein comprises an amino acid substitution in amino acids V20 and D68 as compared to said reference protein. In some embodiments, the modified protein comprises an amino acid substitution in amino acids D68 and R84 as compared to said reference protein.

In some embodiments, the modified protein comprises an amino acid substitution in amino acids D68 and R31 as compared to said reference protein. In some embodiments, the modified protein comprises an amino acid substitution in amino acids R84 and R31 as compared to said reference protein.

In some embodiments, the modified protein comprises an amino acid substitution in amino acids A73 and F89 as compared to said reference protein.

In some embodiments, the modified protein comprises an amino acid substitution in amino acids C41 and T12 as compared to said reference protein.

In some embodiments, the modified protein comprises an amino acid substitution in amino acid V20 and in at least amino acid selected from the group consisting of K25, V64 and D68 as compared to said reference protein. In some embodiments, the modified protein comprises an amino acid substitution in in at least amino acids V20 and V64 as compared to said reference protein. In some embodiments, the modified protein comprises an amino acid substitution in in at least amino acids K25 and V20 as compared to said reference protein. In some embodiments, the modified protein comprises an amino acid substitution in amino acids V20 and C41 and A73 and 175 and F89 as compared to said reference protein.

As previously described by the inventors, modification of amino acids, E2, E23 and Y65, and specifically, amino acid substitutions E2N, E23A and Y65R of the reference protein set forth herein as SEQ ID NO:8, provided a modified protein denoted as DM9 provided by SEQ ID NO:9 that was characterized by improved sweetness and stability as compared to the refence protein.

As shown in the Examples below, modification of residues, E2, E23, Y65, and L70 and specifically, substitutions as E2N, E23A, Y65R, L70I of the reference protein set forth herein as SEQ ID NO:8, provided a modified protein denoted herein as DM14 provided by SEQ ID NO:10 that was suggested to have an improved stability as indicated by a REU value of −321.07 as compared to the REU value of the reference protein of −315.35.

As noted above, it was found that deletions of three amino acids, specifically E50, F52 and R53 improves the stability of the modified protein.

In some embodiments, the modified protein comprises at least the amino acids substitutions selected from the group consisting of E2N, E23A and Y65R.

In accordance with some other aspects, the present disclosure provides a modified protein comprising an amino acid sequence that has amino acid deletions in at least three amino acids located between amino acid T46 and amino acid 156, as compared to a reference protein, and amino acid substitutions in at least five amino acids as compared to a reference protein, wherein the at least five amino acids substitutions comprise substitutions in amino acids E2N, E23A and Y65R and wherein the reference protein has an amino acid sequence set forth in SEQ ID NO:8.

In accordance with some other aspects, the present disclosure provides a modified protein comprising an amino acid sequence that has amino acid deletions in amino acid E50, F52 and R53 as compared to a reference protein and amino acid substitutions in at least five amino acids as compared to a reference protein, wherein the at least five amino acids substitutions comprise substitutions in amino acids E2N, E23A and Y65R as compared to the reference protein and wherein the reference protein has an amino acid sequence set forth in SEQ ID NO:8.

As described herein, a modified protein denoted herein as DM28 set forth by an amino acid sequence SEQ ID NO:40 comprises amino acid deletions in amino acid E50, F52 and R53 and substitutions in amino acids E2N, E23A and Y65R as compared to the reference protein and wherein the reference protein has an amino acid sequence set forth in SEQ ID NO:8. DM28 was shown to have a Tₘ of 91° C. that was an improvement as compared to the reference protein, MNEI (SEQ ID NO:8) that has Tm of 71° C.

In some embodiments, the modified protein comprises at least the amino acids substitutions selected from the group consisting of E2N, E23A, Y65R and L70I.

In accordance with some other aspects, the present disclosure provides a modified protein comprising an amino acid sequence that has amino acid deletions in at least three amino acids located between amino acid T46 and amino acid 156, as compared to a reference protein, and amino acid substitutions in at least five amino acids as compared to a reference protein, wherein the at least five amino acids substitutions comprise substitutions in amino acids E2N, E23A, Y65R and L70I and wherein the reference protein has an amino acid sequence set forth in SEQ ID NO:8.

In accordance with some other aspects, the present disclosure provides a modified protein comprising an amino acid sequence that has amino acid deletions in amino acid E50, F52 and R53 as compared to a reference protein and amino acid substitutions in at least five amino acids as compared to a reference protein, wherein the at least five amino acids substitutions comprise substitutions in amino acids E2N, E23A, Y65R and L70I as compared to the reference protein and wherein the reference protein has an amino acid sequence set forth in SEQ ID NO:8.

As described herein, a modified protein denoted herein as DM31 set forth by an amino acid sequence SEQ ID NO:7 comprises amino acid deletions in amino acid E50, F52 and R53 and substitutions in amino acids E2N, E23A, Y65R and L70I as compared to the reference protein and wherein the reference protein has an amino acid sequence set forth in SEQ ID NO:8. DM31 was shown to have a Tₘ of 91° C. that was an improvement as compared to the reference protein, MNEI (SEQ ID NO:8) that has Tm of 71° C. Further, DM31 has a sweet intensity of 3953, whereas the reference protein has a sweetness intensity 1008.

In some embodiments, the modified protein comprises at least the amino acids substitutions selected from the group consisting of E2N, E23V and Y65K.

In accordance with some other aspects, the present disclosure provides a modified protein comprising an amino acid sequence that has amino acid deletions in at least three amino acids located between amino acid T46 and amino acid 156, as compared to a reference protein, and amino acid substitutions in at least five amino acids as compared to a reference protein, wherein the at least five amino acids substitutions comprise substitutions in amino acids E2N, E23V and Y65K and wherein the reference protein has an amino acid sequence set forth in SEQ ID NO:8.

In accordance with some other aspects, the present disclosure provides a modified protein comprising an amino acid sequence that has amino acid deletions in amino acid E50, F52 and R53 as compared to a reference protein and amino acid substitutions in at least five amino acids as compared to a reference protein, wherein the at least five amino acids substitutions comprise substitutions in amino acids E2N, E23V and Y65K as compared to the reference protein and wherein the reference protein has an amino acid sequence set forth in SEQ ID NO:8.

In some embodiments, the modified protein comprises at least the amino acids substitutions selected from the group consisting of E2N, E23V, Y65K and L70I.

In accordance with some other aspects, the present disclosure provides a modified protein comprising an amino acid sequence that has amino acid deletions in at least three amino acids located between amino acid T46 and amino acid 156, as compared to a reference protein, and amino acid substitutions in at least five amino acids as compared to a reference protein, wherein the at least five amino acids substitutions comprise substitutions in amino acids E2N, E23V, Y65K and L70I and wherein the reference protein has an amino acid sequence set forth in SEQ ID NO:8.

In accordance with some other aspects, the present disclosure provides a modified protein comprising an amino acid sequence that has amino acid deletions in amino acid E50, F52 and R53 as compared to a reference protein and amino acid substitutions in at least five amino acids as compared to a reference protein, wherein the at least five amino acids substitutions comprise substitutions in amino acids E2N, E23V, Y65K and L70I as compared to the reference protein and wherein the reference protein has an amino acid sequence set forth in SEQ ID NO:8.

As described herein, a modified protein denoted herein as DM32 set forth by an amino acid sequence SEQ ID NO:46 comprises amino acid deletions in amino acid E50, F52 and R53 and substitutions in amino acids E2N, E23V, Y65K and L70I as compared to the reference protein and wherein the reference protein has an amino acid sequence set forth in SEQ ID NO:8. DM32 was shown to have a Tₘ of >90° C. that was an improvement as compared to the reference protein, MNEI (SEQ ID NO:8) that has Tm of 71° C.

Without being bound by theory, it was suggested that loop remodeling increases the stability of the modified proteins. It is further suggested that the remodeling leads to extra hydrogen bonds, an elongates and more ordered beta strand, and loop 2 & 3 which were transformed into a beta turn. While the loop is at a specific part of the beta sheet, its role is highly important since the loop is located at the weak point of the beta.

In some embodiments, the modified protein comprises amino acid substitutions (i) E2N, (ii) at least one of E23A or E23V and (iii) at least one of Y65R or Y65K and least one amino acid substitution selected from the group consisting of E4Q, T12V, A19V, V20I, K25R, I26S, I26T, I26W, Q28K, Q28R, Q28K, Q28E, Q28S, R31T, T33R, N35T, C41T, C41V, C41A, C41S, Q61N, V64I, D68S, D68N, D68T, A73V, A73F, 175L, R84L, F89V, and F89M as compared to the reference protein.

In some embodiments, the modified protein comprises amino acid substitutions (i) E2N, (ii) at least one of E23A or E23V, (iii) at least one of Y65R or Y65K, L70I and least one amino acid substitution selected from the group consisting of E4Q, T12V, A19V, V20I, K25R, I26S, I26T, I26W, Q28K, Q28R, Q28K, Q28E, Q28S, R31T, T33R, N35T, C41T, C41V, C41A, C41S, Q61N, V64I, D68S, D68N, D68T, A73V, A73F, 175L, R84L, F89V, and F89M as compared to the reference protein.

In some embodiments, the modified protein comprises amino acid substitutions (i) E2N, (ii) at least one of E23A or E23V, (iii) at least one of Y65R or Y65K and L70I and least one amino acid substitution selected from the group consisting of E4Q, T12V, A19V, V20I, K25R, I26S, I26T, I26W, Q28K, Q28R, Q28K, Q28E, Q28S, R31T, T33R, N35T, C41T, C41V, C41A, C41S, Q61N, V64I, D68S, D68N, D68T, A73V, A73F, 175L, R84L, F89V, and F89M as compared to the reference protein.

In some embodiments, the modified protein comprises amino acid substitutions (i) E2N, (ii) at least one of E23A or E23V and (iii) at least one of Y65R or Y65K and least one amino acid substitution selected from the group consisting of A19V, V20I, K25R, I26S, I26T, I26W, Q28K, Q28R, Q28K, Q28E, Q28S, T33R, N35T, C41T, C41V, C41A, C41S, D68S, D68N, and D68T, as compared to the reference protein.

In some embodiments, the modified protein comprises amino acid substitutions (i) E2N, (ii) at least one of E23A or E23V, (iii) at least one of Y65R or Y65K and L70I and least one amino acid substitution selected from the group consisting of A19V, V20I, K25R, I26S, I26T, I26W, Q28K, Q28R, Q28K, Q28E, Q28S, T33R, N35T, C41T, C41V, C41A, C41S, D68S, D68N, and D68T, as compared to the reference protein.

In some embodiments, the modified protein comprises amino acid substitutions (i) E2N, (ii) at least one of E23A or E23V and (iii) at least one of Y65R or Y65K and least one amino acid substitution selected from the group consisting of A19V, V20I, K25R, Q28K, T33R, C41A, C41S, and D68N, as compared to the reference protein.

In some embodiments, the modified protein comprises amino acid substitutions (i) E2N, (ii) at least one of E23A or E23V, (iii) at least one of Y65R or Y65K and L70I and least one amino acid substitution selected from the group consisting of A19V, V20I, K25R, Q28K, T33R, C41A, C41S, and D68N, as compared to the reference protein.

In some embodiments, the modified protein comprises amino acid substitutions (i) E2N, (ii) at least one of E23A or E23V and (iii) at least one of Y65R or Y65K and least one amino acid substitution selected from the group consisting of A19V, V20I, K25R, Q28K, T33R, C41A, C41S, and D68N, as compared to the reference protein.

In some embodiments, the modified protein comprises amino acid substitutions (i) E2N, (ii) at least one of E23A or E23V, (iii) at least one of Y65R or Y65K and L70I and least one amino acid substitution selected from the group consisting of A19V, V20I, K25R, Q28K, T33R, C41A, C41S, and D68N, as compared to the reference protein.

In some embodiments, the modified protein comprises amino acid substitutions (i) E2N, (ii) at least one of E23A or E23V, (iii) at least one of Y65R or Y65K, L70I and Q28R and at least amino acid selected from the group consisting of V20, K25R, 126 and R31T as compared to said reference protein.

In some embodiments, the modified protein comprises amino acid substitutions (i) E2N, (ii) at least one of E23A or E23V, (iii) at least one of Y65R or Y65K, L70I and Q28K and at least amino acid selected from the group consisting of V20, K25R, I26T, I26W and R31 as compared to said reference protein.

In some embodiments, the modified protein comprises amino acid substitutions (i) E2N, (ii) at least one of E23A or E23V, (iii) at least one of Y65R or Y65K, L70I and Q28E and at least amino acid selected from the group consisting of V20, K25, 126W and R31 as compared to said reference protein.

In some embodiments, the modified protein comprises amino acid substitutions (i) E2N, (ii) at least one of E23A or E23V, (iii) at least one of Y65R or Y65K, L70I and D68N and at least amino acid selected from the group consisting of V20, K25R, R31T and R84L as compared to said reference protein.

In some embodiments, the modified protein comprises amino acid substitutions (i) E2N, (ii) at least one of E23A or E23V, (iii) at least one of Y65R or Y65K, L70I R84L and R31T as compared to said reference protein.

In some embodiments, the modified protein comprises amino acid substitutions (i) E2N, (ii) at least one of E23A or E23V, (iii) at least one of Y65R or Y65K, L70I, A73F and F89M as compared to said reference protein.

In some embodiments, the modified protein comprises amino acid substitutions (i) E2N, (ii) at least one of E23A or E23V, (iii) at least one of Y65R or Y65K, L70I, C41V and T12V as compared to said reference protein.

In some embodiments, the modified protein comprises amino acid substitutions (i) E2N, (ii) at least one of E23A or E23V, (iii) at least one of Y65R or Y65K, L70I and V20I and least amino acid selected from the group consisting of K25, V64I and D68 as compared to said reference protein.

In some embodiments, the modified protein comprises amino acid substitutions (i) E2N, (ii) at least one of E23A or E23V, (iii) at least one of Y65R or Y65K, L70I, V20I and least two amino acids selected from the group consisting of C41A, A73F and I75L and F89M as compared to said reference.

In some embodiments, the modified protein comprises amino acid substitutions (i) E2N, (ii) at least one of E23A or E23V, (iii) at least one of Y65R or Y65K, L70I, and least two amino acids selected from the group consisting of C41S, V64I, A73F, 175L and F89V as compared to said reference.

In some embodiments, the modified protein comprises an amino acid sequence that is at least about 90%, at least about 91%, at least about 92%, at least about 93%, at least about 94%, at least about 95%, at least about 96%, at least about 97%, at least about 98%, or at least about 99% identical to amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:12, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:50, SEQ ID NO:51, SEQ ID NO:52, SEQ ID NO:54, SEQ ID NO:56, SEQ ID NO:57, SEQ ID NO:58, SEQ ID NO:59, SEQ ID NO:60, SEQ ID NO:63, SEQ ID NO:64, SEQ ID NO:65, SEQ ID NO:66, SEQ ID NO:67, SEQ ID NO:68, SEQ ID NO:69 and SEQ ID NO:71.

In some embodiments, the modified protein comprises an amino acid sequence that is between about 90% and about 99% identical to amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:12, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:50, SEQ ID NO:51, SEQ ID NO:52, SEQ ID NO:54, SEQ ID NO:56, SEQ ID NO:57, SEQ ID NO:58, SEQ ID NO:59, SEQ ID NO:60, SEQ ID NO:63, SEQ ID NO:64, SEQ ID NO:65, SEQ ID NO:66, SEQ ID NO:67, SEQ ID NO:68, SEQ ID NO:69 and SEQ ID NO:71.

In some embodiments, the modified protein comprises an amino acid sequence that is about 90%, about 91%, about 92%, about 93%, about 94%, about 95%, about 96%, about 97%, about 98%, or about 99% identical to amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:12, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:50, SEQ ID NO:51, SEQ ID NO:52, SEQ ID NO:54, SEQ ID NO:56, SEQ ID NO:57, SEQ ID NO:58, SEQ ID NO:59, SEQ ID NO:60, SEQ ID NO:63, SEQ ID NO:64, SEQ ID NO:65, SEQ ID NO:66, SEQ ID NO:67, SEQ ID NO:68, SEQ ID NO:69 and SEQ ID NO:71.

Sequence similarity or sequence homology as used herein refers to the amount (%) of amino acids that are conserved with similar physicochemical properties, e.g., leucine and isoleucine.

In determining the sequence identity, gaps are not counted and sequence identity is relative to the shorter sequence of the two. In this context, it should be noted that the length of the reference MNEI protein (amino acid sequence) may be the same as the modified MNEI protein (amino acid sequence) or may be different from the modified MNEI protein (amino acid sequence).

The term “amino acid sequence” and/or “polypeptide chain” are used to describe a protein having an amino acid sequence or polypeptide chain. As such, the term “reference protein” is equivalent to the term “reference amino acid sequence,” and the term “modified protein” is equivalent to the term “modified amino acid sequence.” It should be noted that the terms “amino acid sequence” and/or “polypeptide chain” encompass sequences having a 3D structure as well as sequences with no 3D structure.

The term “fragment” as used herein in connection with the disclosure relates to proteins or peptides derived from full-length proteins that are shortened, i.e., lacking at least one amino acid. Such fragments may include at least 10, more such as 20, or 30 or more consecutive amino acids of the protein's primary sequence.

In some embodiments, the modified protein comprises an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:12, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:50, SEQ ID NO:51, SEQ ID NO:52, SEQ ID NO:54, SEQ ID NO:56, SEQ ID NO:57, SEQ ID NO:58, SEQ ID NO:59, SEQ ID NO:60, SEQ ID NO:63, SEQ ID NO:64, SEQ ID NO:65, SEQ ID NO:66, SEQ ID NO:67, SEQ ID NO:68, SEQ ID NO:69 and SEQ ID NO:71 or of a fragment or variant thereof.

In some embodiments, the modified protein comprises an amino acid sequence selected from the group consisting of SEQ ID NO:5, SEQ ID NO:14, SEQ ID NO:17, SEQ ID NO:25, SEQ ID NO:34, SEQ ID NO:36, SEQ ID NO:38 and SEQ ID NO:49, or of a fragment or variant thereof.

In some embodiments, the modified protein is or comprises an amino acid sequence denoted by any one of DM42 (SEQ ID NO:4), DM43 (SEQ ID NO:5), DM41 (SEQ ID NO:12), DM65 (SEQ ID NO:14), DM66 (SEQ ID NO:15), DM68 (SEQ ID NO:16), DM69 (SEQ ID NO:17), DM73 (SEQ ID NO:18), DM92 (SEQ ID NO:19), DM115 (SEQ ID NO:21), DM116 (SEQ ID NO:22), DM144 (SEQ ID NO:23) or DM145 (SEQ ID NO:24).

In some embodiments, the modified protein is or comprises an amino acid sequence denoted by any one of DM46 (SEQ ID NO:1), DM91 (SEQ ID NO:28), DM143 (SEQ ID NO:44), DM150 (SEQ ID NO:32), DM151 (SEQ ID NO:33).

In some embodiments, the modified protein is or comprises an amino acid sequence denoted by any one of DM46 (SEQ ID NO:1), DM77 (SEQ ID NO:25), DM84 (SEQ ID NO:26), DM85 (SEQ ID NO:27), DM91 (SEQ ID NO:28), DM96 (SEQ ID NO:38), DM103 (SEQ ID NO:20), DM104 (SEQ ID NO:29), DM131 (SEQ ID NO:30), DM132 (SEQ ID NO:31), DM143 (SEQ ID NO:44), DM150 (SEQ ID NO:32), DM151 (SEQ ID NO:33), DM152 (SEQ ID NO:45).

In some embodiments, the modified protein is or comprises an amino acid sequence denoted by any one DM57 (SEQ ID NO:34), DM75 (SEQ ID NO:35).

In some embodiments, the modified protein is or comprises an amino acid sequence denoted by any one of DM87 (SEQ ID NO:36), DM93 (SEQ ID NO:37), DM157 (SEQ ID NO:39 used as reference).

In some embodiments, the modified protein is or comprises an amino acid sequence denoted by any one of DM47 (SEQ ID NO:2), DM72 (SEQ ID NO:6).

In some embodiments, the modified protein is or comprises an amino acid sequence denoted by DM70 (SEQ ID NO:3).

In some embodiments, the modified protein is or comprises an amino acid sequence denoted by SEQ ID NO:1 (DM46). In some embodiments, the modified protein is or comprises an amino acid sequence denoted by SEQ ID NO:2 (DM47). In some embodiments, the modified protein is or comprises an amino acid sequence denoted by SEQ ID NO:3 (DM70). In some embodiments, the modified protein is or comprises an amino acid sequence denoted by SEQ ID NO:4 (DM42). In some embodiments, the modified protein is or comprises an amino acid sequence denoted by SEQ ID NO:5 (DM43). In some embodiments, the modified protein is or comprises an amino acid sequence denoted by SEQ ID NO:6 (DM72). In some embodiments, the modified protein is or comprises an amino acid sequence denoted by SEQ ID NO:12 (DM41). In some embodiments, the modified protein is or comprises an amino acid sequence denoted by SEQ ID NO:14 (DM65). In some embodiments, the modified protein is or comprises an amino acid sequence denoted by SEQ ID NO:15 (DM66). In some embodiments, the modified protein is or comprises an amino acid sequence denoted by SEQ ID NO:16 (DM68). In some embodiments, the modified protein is or comprises an amino acid sequence denoted by SEQ ID NO:17 (DM69). In some embodiments, the modified protein is or comprises an amino acid sequence denoted by SEQ ID NO:18 (DM73). In some embodiments, the modified protein is or comprises an amino acid sequence denoted by SEQ ID NO:19 (DM92). In some embodiments, the modified protein is or comprises an amino acid sequence denoted by SEQ ID NO:20 (DM103). In some embodiments, the modified protein is or comprises an amino acid sequence denoted by SEQ ID NO:21 (DM115). In some embodiments, the modified protein is or comprises an amino acid sequence denoted by SEQ ID NO:22 (DM116). In some embodiments, the modified protein is or comprises an amino acid sequence denoted by SEQ ID NO:23 (DM144). In some embodiments, the modified protein is or comprises an amino acid sequence denoted by SEQ ID NO:24 (DM145). In some embodiments, the modified protein is or comprises an amino acid sequence denoted by SEQ ID NO:25 (DM77). In some embodiments, the modified protein is or comprises an amino acid sequence denoted by SEQ ID NO:26 (DM84). In some embodiments, the modified protein is or comprises an amino acid sequence denoted by SEQ ID NO:27 (DM85). In some embodiments, the modified protein is or comprises an amino acid sequence denoted by SEQ ID NO:28 (DM91). In some embodiments, the modified protein is or comprises an amino acid sequence denoted by SEQ ID NO:29 (DM104). In some embodiments, the modified protein is or comprises an amino acid sequence denoted by SEQ ID NO:30 (DM131). In some embodiments, the modified protein is or comprises an amino acid sequence denoted by SEQ ID NO:31 (DM132). In some embodiments, the modified protein is or comprises an amino acid sequence denoted by SEQ ID NO:32 (DM150). In some embodiments, the modified protein is or comprises an amino acid sequence denoted by SEQ ID NO:33 (DM151). In some embodiments, the modified protein is or comprises an amino acid sequence denoted by SEQ ID NO:34 (DM57). In some embodiments, the modified protein is or comprises an amino acid sequence denoted by SEQ ID NO:35 (DM75). In some embodiments, the modified protein is or comprises an amino acid sequence denoted by SEQ ID NO:36 (DM87). In some embodiments, the modified protein is or comprises an amino acid sequence denoted by SEQ ID NO:37 (DM93). In some embodiments, the modified protein is or comprises an amino acid sequence denoted by SEQ ID NO:38 (DM96). In some embodiments, the modified protein is or comprises an amino acid sequence denoted by SEQ ID NO:41 (DM94). In some embodiments, the modified protein is or comprises an amino acid sequence denoted by SEQ ID NO:42 (DM97). In some embodiments, the modified protein is or comprises an amino acid sequence denoted by SEQ ID NO:43 (DM99). In some embodiments, the modified protein is or comprises an amino acid sequence denoted by SEQ ID NO:44 (DM143). In some embodiments, the modified protein is or comprises an amino acid sequence denoted by SEQ ID NO:45 (DM152). In some embodiments, the modified protein is or comprises an amino acid sequence denoted by SEQ ID NO:46 (DM32). In some embodiments, the modified protein is or comprises an amino acid sequence denoted by SEQ ID NO:47 (DM33). In some embodiments, the modified protein is or comprises an amino acid sequence denoted by SEQ ID NO:48 (DM61). In some embodiments, the modified protein is or comprises an amino acid sequence denoted by SEQ ID NO:50 (DM108). In some embodiments, the modified protein is or comprises an amino acid sequence denoted by SEQ ID NO:51 (DM117). In some embodiments, the modified protein is or comprises an amino acid sequence denoted by SEQ ID NO:52 (DM330). In some embodiments, the modified protein is or comprises an amino acid sequence denoted by SEQ ID NO:54 (DM164). In some embodiments, the modified protein is or comprises an amino acid sequence denoted by SEQ ID NO:56 (DM491). In some embodiments, the modified protein is or comprises an amino acid sequence denoted by SEQ ID NO:57 (DM498). In some embodiments, the modified protein is or comprises an amino acid sequence denoted by SEQ ID NO:58 (DM506). In some embodiments, the modified protein is or comprises an amino acid sequence denoted by SEQ ID NO:59 (DM508). In some embodiments, the modified protein is or comprises an amino acid sequence denoted by SEQ ID NO:60 (DM509). In some embodiments, the modified protein is or comprises an amino acid sequence denoted by SEQ ID NO:63 (DM341). In some embodiments, the modified protein is or comprises an amino acid sequence denoted by SEQ ID NO:64 (DM420). In some embodiments, the modified protein is or comprises an amino acid sequence denoted by SEQ ID NO:65 (DM424). In some embodiments, the modified protein is or comprises an amino acid sequence denoted by SEQ ID NO:66 (DM432). In some embodiments, the modified protein is or comprises an amino acid sequence denoted by SEQ ID NO:67 (DM452). In some embodiments, the modified protein is or comprises an amino acid sequence denoted by SEQ ID NO:68 (DM489). In some embodiments, the modified protein is or comprises an amino acid sequence denoted by SEQ ID NO:69 (DM505). In some embodiments, the modified protein is or comprises an amino acid sequence denoted by SEQ ID NO:71 (DM510).

In some examples, the modified protein is at least one of the proteins shown in Table 20. It should be noted that the present disclosure encompasses combinations of two or more of the modified proteins shown in Table 20. It should be further noted that each row in Table 20 constitutes an embodiment of the present disclosure.

TABLE 20
List of modified proteins, the respective
SEQ ID NO the amino acid sequence

| | SEQ ID | |
|---|---|---|
| DM# | NO: | Amino acid sequence |
| DM46 | SEQ ID | GNWEIIDIGPFTQNLGKFAVDEANKIGQYGR |
| | NO: 1 | LTFNKVIRPTMKKTIYENGEIKGYEYQLYVR |
| | | ASDKIFRADISEDYKTRGRKLLRFNGPVPPP |
| DM47 | SEQ ID | GNWEIIDIGPFTQNLGKFAVDEANKIGQYGT |
| | NO: 2 | LTFNKVIRPCMKKTIYENGEIKGYEYQLYVR |
| | | ASDKIFRADISEDYKTRGRKLLRFNGPVPPP |
| DM70 | SEQ ID | GNWEIIDIGPFTQNLGKFAVDEANKIGQYGR |
| | NO: 3 | LTFNKVIRPCMKKTIYENGEIKGYEYQLYVR |
| | | ASDKIFRADISEDYKTRGLKLLRFNGPVPPP |
| DM42 | SEQ ID | GNWEIIDIGPFTQNLGKFAVDEANKTGQYGR |
| | NO: 4 | LTFNKVIRPCMKKTIYENGEIKGYEYQLYVR |
| | | ASDKIFRADISEDYKTRGRKLLRFNGPVPPP |
| DM43 | SEQ ID | GNWEIIDIGPFTQNLGKFAVDEANRIGQYGR |
| | NO: 5 | LTFNKVIRPCMKKTIYENGEIKGYEYQLYVR |
| | | ASDKIFRADISEDYKTRGRKLLRFNGPVPPP |
| DM72 | SEQ ID | GNWEIIDIGPFTQNLGKFAVDEANKIGQYGR |
| | NO: 6 | LTFTKVIRPCMKKTIYENGEIKGYEYQLYVR |
| | | ASDKIFRADISEDYKTRGRKLLRFNGPVPPP |
| DM31 | SEQ ID | GNWEIIDIGPFTQNLGKFAVDEANKIGQYGR |
| | NO: 7 | LTFNKVIRPCMKKTIYENGEIKGYEYQLYVR |
| | | ASDKIFRADISEDYKTRGRKLLRFNGPVPPP |
| MNEI | SEQ ID | GEWEIIDIGPFTQNLGKFAVDEENKIGQYGR |
| | NO: 8 | LTFNKVIRPCMKKTIYENEGFREIKGYEYQL |
| | | YVYASDKLFRADISEDYKTRGRKLLRFNGPV |
| | | PPP |
| DM09 | SEQ ID | GNWEIIDIGPFTQNLGKFAVDEANKIGQYGR |
| | NO: 9 | LTFNKVIRPCMKKTIYENEGFREIKGYEYQL |
| | | YVRASDKLFRADISEDYKTRGRKLLRFNGPV |
| | | PPP |
| DM14 | SEQ ID | GNWEIIDIGPFTQNLGKFAVDEANKIGQYGR |
| | NO: 10 | LTFNKVIRPCMKKTIYENEGFREIKGYEYQL |
| | | YVRASDKIFRADISEDYKTRGRKLLRFNGPV |
| | | PPP |
| DM29 | SEQ ID | GEWEIIDIGPFTQNLGKFAVDEENKIGQYGR |
| | NO: 11 | LTFNKVIRPCMKKTIYENGEIKGYEYQLYVY |
| | | ASDKLFRADISEDYKTRGRKLLRFNGPVPPP |
| DM41 | SEQ ID | GNWEIIDIGPFTQNLGKFAVDEANKSGQYGR |
| | NO: 12 | LTFNKVIRPCMKKTIYENGEIKGYEYQLYVR |
| | | ASDKIFRADISEDYKTRGRKLLRFNGPVPPP |
| DM42b | SEQ ID | GEWEIIDIGPFTQNLGKFAVDEENKTGQYGR |
| | NO: 13 | LTFNKVIRPCMKKTIYENEGFREIKGYEYQL |
| | | YVYASDKLFRADISEDYKTRGRKLLRFNGPV |
| | | PPP |
| DM65 | SEQ ID | GNWEIIDIGPFTQNLGKFAVDEANKIGKYGR |
| | NO: 14 | LTFNKVIRPCMKKTIYENGEIKGYEYQLYVR |
| | | ASDKIFRADISEDYKTRGRKLLRFNGPVPPP |
| DM66 | SEQ ID | GNWEIIDIGPFTQNLGKFAVDEANKIGRYGR |
| | NO: 15 | LTFNKVIRPCMKKTIYENGEIKGYEYQLYVR |
| | | ASDKIFRADISEDYKTRGRKLLRFNGPVPPP |
| DM68 | SEQ ID | GNWEIIDIGPFTQNLGKFAVDEANRIGRYGR |
| | NO: 16 | LTFNKVIRPCMKKTIYENGEIKGYEYQLYVR |
| | | ASDKIFRADISEDYKTRGRKLLRFNGPVPPP |
| DM69 | SEQ ID | GNWEIIDIGPFTQNLGKFAVDEANRIGKYGR |
| | NO: 17 | LTFNKVIRPCMKKTIYENGEIKGYEYQLYVR |
| | | ASDKIFRADISEDYKTRGRKLLRFNGPVPPP |
| DM73 | SEQ ID | GNWEIIDIGPFTQNLGKFAVDEANKIGRYGT |
| | NO: 18 | LTFNKVIRPCMKKTIYENGEIKGYEYQLYVR |
| | | ASDKIFRADISEDYKTRGRKLLRFNGPVPPP |
| DM92 | SEQ ID | GNWEIIDIGPFTQNLGKFAVDEANKTGKYGR |
| | NO: 19 | LTFNKVIRPCMKKTIYENGEIKGYEYQLYVR |
| | | ASDKIFRADISEDYKTRGRKLLRFNGPVPPP |
| DM103 | SEQ ID | GNWEIIDIGPFTQNLGKFAVDEANKIGQYGR |
| | NO: 20 | LTFNKVIRPCMKKTIYENGEIKGYEYQLYVR |
| | | ASDKIFRADLSEDYKTRGRKLLRFNGPVPPP |
| DM115 | SEQ ID | GNWEIIDIGPFTQNLGKFAVDEANKWGEYGR |
| | NO: 21 | LTFNKVIRPCMKKTIYENGEIKGYEYQLYVR |
| | | ASDKIFRADISEDYKTRGRKLLRFNGPVPPP |
| DM116 | SEQ ID | GNWEIIDIGPFTQNLGKFAVDEANKWGKYGR |
| | NO: 22 | LTFNKVIRPCMKKTIYENGEIKGYEYQLYVR |
| | | ASDKIFRADISEDYKTRGRKLLRFNGPVPPP |
| DM144 | SEQ ID | GNWEIIDIGPFTQNLGKFAVDEANKWGQYGR |
| | NO: 23 | LTFNKVIRPCMKKTIYENGEIKGYEYQLYVR |
| | | ASDKIFRADISEDYKTRGRKLLRFNGPVPPP |
| DM145 | SEQ ID | GNWEIIDIGPFTQNLGKFAVDEANKIGEYGR |
| | NO: 24 | LTFNKVIRPCMKKTIYENGEIKGYEYQLYVR |
| | | ASDKIFRADISEDYKTRGRKLLRFNGPVPPP |
| DM77 | SEQ ID | GNWEIIDIGPFTQNLGKFVVDEANKIGQYGR |
| | NO: 25 | LTFNKVIRPCMKKTIYENGEIKGYEYQLYVR |
| | | ASDKIFRADISEDYKTRGRKLLRFNGPVPPP |
| DM84 | SEQ ID | GNWEIIDIGPFTQNLGKFAVDEANKIGQYGR |
| | NO: 26 | LTFNKVIRPCMKKTIYENGEIKGYEYQLYIR |
| | | ASDKIFRADISEDYKTRGRKLLRFNGPVPPP |
| DM85 | SEQ ID | GNWEIIDIGPFTQNLGKFAVDEANKIGQYGR |
| | NO: 27 | LTFNKVIRPCMKKTIYENGEIKGYEYQLYVR |
| | | ASDKIFRVDISEDYKTRGRKLLRFNGPVPPP |
| DM91 | SEQ ID | GNWEIIDIGPFTQNLGKFAVDEANKIGQYGR |
| | NO: 28 | LTFNKVIRPVMKKTIYENGEIKGYEYQLYVR |
| | | ASDKIFRADISEDYKTRGRKLLRFNGPVPPP |
| DM104 | SEQ ID | GNWEIIDIGPFTQNLGKFAVDEANKIGQYGR |
| | NO: 29 | LTFNKVIRPCMKKTIYENGEIKGYEYQLYVR |
| | | ASDKIFRADISEDYKTRGRKLLRMNGPVPPP |
| DM131 | SEQ ID | GNWEIIDIGPFTQNLGKFAVDEANKIGQYGR |
| | NO: 30 | LTFNKVIRPCMKKTIYENGEIKGYEYQLYVR |
| | | ASDKIFRFDISEDYKTRGRKLLRFNGPVPPP |
| DM132 | SEQ ID | GNWEIIDIGPFTQNLGKFAVDEANKIGQYGR |
| | NO: 31 | LTFNKVIRPCMKKTIYENGEIKGYEYQLYVR |
| | | ASDKIFRFDISEDYKTRGRKLLRMNGPVPPP |
| DM150 | SEQ ID | GNWEIIDIGPFTQNLGKFAVDEANKIGQYGR |
| | NO: 32 | LTFNKVIRPAMKKTIYENGEIKGYEYQLYVR |
| | | ASDKIFRADISEDYKTRGRKLLRFNGPVPPP |
| DM151 | SEQ ID | GNWEIIDIGPFTQNLGKFAVDEANKIGQYGR |
| | NO: 33 | LTFNKVIRPSMKKTIYENGEIKGYEYQLYVR |
| | | ASDKIFRADISEDYKTRGRKLLRFNGPVPPP |
| DM57 | SEQ ID | GNWEIIDIGPFTQNLGKFAVDEANKIGQYGR |
| | NO: 34 | LRFNKVIRPCMKKTIYENGEIKGYEYQLYVR |
| | | ASDKIFRADISEDYKTRGRKLLRFNGPVPPP |
| DM75 | SEQ ID | GNWQIIDIGPFTQNLGKFAVDEANKIGQYGR |
| | NO: 35 | LTFNKVIRPCMKKTIYENGEIKGYEYQLYVR |
| | | ASDKIFRADISEDYKTRGRKLLRFNGPVPPP |
| DM87 | SEQ ID | GNWEIIDIGPFTQNLGKFAVDEANKIGQYGR |
| | NO: 36 | LTFNKVIRPCMKKTIYENGEIKGYEYQLYVR |
| | | ASNKIFRADISEDYKTRGRKLLRFNGPVPPP |
| DM93 | SEQ ID | GNWEIIDIGPFTQNLGKFAVDEANKIGQYGR |
| | NO: 37 | LTFNKVIRPCMKKTIYENGEIKGYEYQLYVR |
| | | ASNKIFRADISEDYKTRGLKLLRFNGPVPPP |
| DM96 | SEQ ID | GNWEIIDIGPFTQNLGKFAIDEANKIGQYGR |
| | NO: 38 | LTFNKVIRPCMKKTIYENGEIKGYEYQLYVR |
| | | ASDKIFRADISEDYKTRGRKLLRFNGPVPPP |
| DM157 | SEQ ID | GEWEIIDIGPFTQNLGKFAVDEENKIGQYGR |
| | NO: 39 | LTFNKVIRPCMKKTIYENEGFREIKGYEYQL |
| | | YVYASNKLFRADISEDYKTRGRKLLRFNGPV |
| | | PPP |
| DM28 | SEQ ID | GNWEIIDIGPFTQNLGKFAVDEANKIGQYGR |
| | NO: 40 | LTFNKVIRPCMKKTIYENGEIKGYEYQLYVR |
| | | ASDKLFRADISEDYKTRGRKLLRFNGPVPPP |
| DM94 | SEQ ID | GNWEIIDIGPFTQNLGKFAVDEANKIGQYGT |
| | NO: 41 | LTFNKVIRPCMKKTIYENGEIKGYEYQLYVR |
| | | ASNKIFRADISEDYKTRGRKLLRFNGPVPPP |
| DM97 | SEQ ID | GNWEIIDIGPFTQNLGKFAVDEANKIGQYGT |
| | NO: 42 | LTFNKVIRPCMKKTIYENGEIKGYEYQLYVR |
| | | ASDKIFRADISEDYKTRGLKLLRFNGPVPPP |
| DM99 | SEQ ID | GNWEIIDIGPFTQNLGKFAVDEANKIGQYGR |
| | NO: 43 | LTFNKVIRPCMKKTIYENGEIKGYEYNLYVR |
| | | ASDKIFRADISEDYKTRGRKLLRFNGPVPPP |
| DM143 | SEQ ID | GNWEIIDIGPFVQNLGKFAVDEANKIGQYGR |
| | NO: 44 | LTFNKVIRPVMKKTIYENGEIKGYEYQLYVR |
| | | ASDKIFRADISEDYKTRGRKLLRFNGPVPPP |
| DM152 | SEQ ID | GNWEIIDIGPFVQNLGKFAVDEANKIGQYGR |
| | NO: 45 | LTFNKVIRPCMKKTIYENGEIKGYEYQLYVR |
| | | ASDKIFRADISEDYKTRGRKLLRFNGPVPPP |
| DM32 | SEQ ID | GNWEIIDIGPFTQNLGKFAVDEVNKIGQYGR |
| | NO: 46 | LTFNKVIRPCMKKTIYENGEIKGYEYQLYVK |
| | | ASDKIFRADISEDYKTRGRKLLRFNGPVPPP |
| DM33 | SEQ ID | GNWEIIDIGPFTQNLGKFAVDEVNKIGQYGR |
| | NO: 47 | LTFNTVIRPCMKKTIYENGEIKGYEYQLYVK |
| | | ASDKIFRADISEDYKTRGRKLLRFNGPVPPP |
| DM61 | SEQ ID | GNWEIIDIGPFTQNLGKFAVDEANKIGQYGR |
| | NO: 48 | LTFNKVIRPCMKKTIYENGEIKGYEYQLYVR |
| | | ASSKIFRADISEDYKTRGRKLLRFNGPVPPP |
| DM108 | SEQ ID | GNWEIIDIGPFTQNLGKFAVDEANKIGQYGR |
| | NO: 50 | LTFNKVIRPCMKKTIYENGEIKGYEYQLYVR |
| | | ASTKIFRADISEDYKTRGRKLLRFNGPVPPP |
| DM117 | SEQ ID | GNWEIIDIGPFTQNLGKFAIDEANKIGQYGR |
| | NO: 51 | LTFNKVIRPCMKKTIYENGEIKGYEYQLYIR |
| | | ASDKIFRADISEDYKTRGRKLLRFNGPVPPP |
| DM330 | SEQ ID | GNWEIIDIGPFTQNLGKFAVDEANKIGSYGR |
| | NO: 52 | LTFNKVIRPCMKKTIYENGEIKGYEYQLYVR |
| | | ASDKIFRADISEDYKTRGRKLLRFNGPVPPP |
| DM464 | SEQ ID | GNWEIIDIGPFTQNLGKFAVDEANRIGQYGR |
| | NO: 54 | LTFNKVIRPCMKKTIYENGEIKGYEYQLYVR |
| | | ASNKIFRADISEDYKTRGRKLLRFNGPVPPP |
| DM491 | SEQ ID | GNWEIIDIGPFTQNLGKFAIDEANKIGQYGR |
| | NO: 56 | LTFNKVIRPAMKKTIYENGEIKGYEYQLYVR |
| | | ASDKIFRFDLSEDYKTRGRKLLRMNGPVPPP |
| DM498 | SEQ ID | GNWEIIDIGPFTQNLGKFAVDEANKIGQYGR |
| | NO: 57 | LTFNKVIRPSMKKTIYENGEIKGYEYQLYIR |
| | | ASDKIFRFDLSEDYKTRGRKLLRVNGPVPPP |
| DM506 | SEQ ID | GNWEIIDIGPFTQNLGKFAIDEANKIGQYGR |
| | NO: 58 | LTFNKVIRPCMKKTIYENGEIKGYEYQLYVR |
| | | ASNKIFRADISEDYKTRGRKLLRFNGPVPPP |
| DM508 | SEQ ID | GNWEIIDIGPFTQNLGKFAIDEANKIGKYGR |
| | NO: 59 | LTFNKVIRPCMKKTIYENGEIKGYEYQLYVR |
| | | ASDKIFRADISEDYKTRGRKLLRFNGPVPPP |
| DM509 | SEQ ID | GNWEIIDIGPFTQNLGKFAIDEANRIGQYGR |
| | NO: 60 | LTFNKVIRPCMKKTIYENGEIKGYEYQLYVR |
| | | ASDKIFRADISEDYKTRGRKLLRFNGPVPPP |
| DM341 | SEQ ID | GNWEIIDIGPFTQNLGKFAVDEANKIGQYGR |
| | NO: 63 | LTFNTVIRPCMKKTIYENGEIKGYEYQLYVR |
| | | ASDKIFRADISEDYKTRGRKLLRFNGPVPPP |
| DM420 | SEQ ID | GNWEIIDIGPFTQNLGKFAVDEANKIGQYGR |
| | NO: 64 | LTFNKVIRPCMKKTIYENGEIKGYEYQLYVR |
| | | ASDKIFRADISEDYKTRGYKLLRFNGPVPPP |
| DM424 | SEQ ID | GNWEIIDIGPFTQNLGKFAVDEANKIGQYGR |
| | NO: 65 | LTFNKVIRPCMKKTIYENGEIKGYEYQLYVR |
| | | ASDKIFRADISEDYKTRGIKLLRFNGPVPPP |
| DM432 | SEQ ID | GNWEIIDIGPFTQNLAKFAVDEANKIGQYGR |
| | NO: 66 | LTFNKVIRPAMKKTIYENGEIKGYEYQLYLR |
| | | ASDKIFRADVSEDYKTRGRKLLRFNGPVPPP |
| DM452 | SEQ ID | GNWEIIDIGPFTQNLGKFAVNEANKIGQYGR |
| | NO: 67 | LTFNKVIRPCMKKTIYENGEIKGYEYQLYVR |
| | | ASDKIFRADISEDYKTRGRKLLRFNGPVPPP |
| DM489 | SEQ ID | GNWEIIDIGPFTQNLGKFAIDEANKIGQYGR |
| | NO: 68 | LTFNKVIRPVMKKTIYENGEIKGYEYQLYVR |
| | | ASDKIFRVDISEDYKTRGRKLLRFNGPVPPP |
| DM505 | SEQ ID | GNWEIIDIGPFTQNLGKFVVDEANKIGKYGR |
| | NO: 69 | LTFNKVIRPCMKKTIYENGEIKGYEYQLYVR |
| | | ASDKIFRADISEDYKTRGRKLLRFNGPVPPP |
| DM510 | SEQ ID | GNWEIIDIGPFTQNLGKFVVDEANRIGQYGR |
| | NO: 71 | LTFNKVIRPCMKKTIYENGEIKGYEYQLYVR |
| | | ASDKIFRADISEDYKTRGRKLLRFNGPVPPP |

As described herein, design of the modified protein is done using computational tools or by expert protein design and structural biology methods, e.g., site-directed mutagenesis, protein engineering, or directed evolution, as further described below. The inventors have developed computational methodologies based on sequence data, structural data, and/or evolutionary data of the reference flavor proteins and other proteins that have local or global similarities to the reference flavor protein in sequence and/or structural features. The computational methods developed and applied herein enabled the inventors to design proteins with specific amino acid substitutions that are energetically favorable and thus are predicted to have improved traits such as thermostability, halostability, pH-stability, shelf-life, folding, and solubility features. Specifically, Computational Protein Design (CPD) was applied to specific sites or regions within the reference protein structure and/or sequence that are not necessary for functional binding to the receptor. In addition, CPD allowed the inventors to limit the substitutions to a predefined set of amino acids that fit the required improved features. The predefined set of amino acids is both in the input data, i.e., the regions of the protein subjected to CPD, and in the output data, i.e., the location and types of amino acids present in the resulting modified protein.

For example, by using CPD it is possible to replace “non-ideal” amino acids (such as hydrophilic amino acids within a hydrophobic core or hydrophobic amino acids on the external surface region) with “ideal” amino acids (such as hydrophilic amino acids in the external surface region and hydrophobic amino acids within a hydrophobic core).

Without being bound by theory, the inventors suggest that substituting hydrophobic amino acids with hydrophilic amino acids on the external surface region will reduce non-specific binding to the oral cavity and reduce the lingering aftertaste.

The methodologies developed herein comprise searching for “stabilizing substitutions,” e.g., amino acid substitutions that will decrease the protein structure's overall energy. The overall energy may be calculated by applying known algorithms in the art. Non-limiting examples of such algorithms include Rosetta, OSPREY (M. Hallen, J. Martin, et al., Journal of Computational Chemistry 2018; 39(30): 2494-2507), or EnCoM (Frappier V, Chartier M, Najmanovich R J. Nucleic Acids Res. 2015; 43(W1): W395-400). These CPD methods undergo focusing and filtering by an array of orthogonal methods such as evolutionary sequence and structural consensus, regular and high-temperature molecular dynamics (MD) and other dynamic simulations, correlated mutational analysis (CMA), surface electrostatics analysis, visual inspection, as well as analysis of cavities, hydrophobic patches, unsatisfied hydrogen bonds and alike.

The amino acid substitutions are based on the following considerations: (a) surface electrostatic potential and (lack of) hydrophobic patches on the surface, (b) retention of the protein's isoelectric point (pI) in a specific range, (c) analysis of the intra-protein cavities, (d) dynamic stability including correlated mutational analysis, normal mode analysis, and root mean square fluctuations (RMSF) in high-temperature or room temperature dynamics, (e) entropic and/or enthalpic components of the substitution energetics, (f) visualization of the specific substitution, (g) types of amino-acids permitted in the family of related proteins; as reflected by an evolutionary conservation analysis of a curated multiple sequence alignment (MSA), and (h) frequency of the substitution as reflected in low-pseudo-energy CPD calculations.

The computational methodologies include one or more of the following steps:
(1) Multiple Sequence Alignment (MSA) or Multiple Structural Alignment. In this step, DNA sequences and/or protein sequences with similarity to the target reference protein or fragments thereof are queried in public databases. Based on the obtained results, a multiple-sequence alignment (MSA) or multiple structural alignment is constructed and the conservation rate is calculated. According to MSA results, a decision regarding the level of CPD to be conducted is made. In non-conserved positions, all amino acids (with or without Cysteine) are allowed in CPD, whereas for more conserved positions, the CPD is limited to residues with similar properties (charge, size, internal dynamics, etc.). This step involves limiting the substitutions in each position based on biophysical knowledge and conservation data. The MSA may yield a Position Specific Substitution Matrix (PSSM) in which each location along the sequence is described in a way correlated with the relative abundance of each amino acid possibly taking into account a potential probability of substitution or of deletion or insertion of amino acids.
(2) Protein function analysis and analysis of structure-function-dynamics relationships. In this step, a database of substitutions with known impact (such as on activity, structure, binding, etc.) is constructed using prior knowledge. Substitutions and substitution-adjacent positions (e.g., a distance of 0.5-1 nm), known, based on prior knowledge, to disturb protein stability and/or function are limited during CPD and are not substituted.
(3) CPD. This step is partially done by designated software such as ROSETTA, OSPREY, SCWRL, PyMol, AlphaFold and alike. Before deterministic CPD is conducted, the reference protein 3D structure/model is energy minimized. The CPD may include site-directed amino-acid replacement where one amino-acid is replaced by another or replacement of protein regions by other amino-acid sequences such resulting in a protein with the different length. The latter can be done by rebuilding regions such as loop by ab initio methods or by taking regions from other proteins, a method that may be referred to as ‘grafting’. For each reference protein, multiple models are considered.
(4) Selection: The models of proteins with the lowest energy are collected. An MSA is assembled on these models, and a conserved sequence is determined. Based on biochemical and biophysical pre-knowledge, subsets of substitutions are chosen. These subsets represent replacements at one or more positions that appear during CPD in high frequency. Each subset is then modeled on a 3D structure of the protein and energy minimized. The lowest energy subset is then selected for further computational and experimental validation.

One of the considerations used in CPD is the receptor binding site and whether to substitute amino acids in the binding region and its proximity. Determining the amino acids residues crucial to taste receptor binding may be generally done by single point substitution of various amino acids. As detailed here in the Examples, the inventors have used computational analysis for characterizing the putative binding sites to the taste receptor. The inventors have identified several novel binding sites in the taste receptor that bind to the reference and the modified proteins.

Another consideration in CPD is retaining the functional plasticity required for binding to the receptor while increasing the thermal stability, which intrinsically is often associated with protein rigidification. The protein must undergo some conformational changes (also known as ‘functional plasticity’) in order to activate the receptor. The inventors thus focused on regions which can be rigidified while conserving regions where functional plasticity must be retained.

As shown herein, CPD analysis have identified specific amino acid residues/regions in MNEI protein which can be modified in order to obtain a modified protein with improved properties, including, inter alia, stability and taste.

For example, MD simulations of MNEI have suggested that the beta sheet of MNEI is less stable. MNEI loses his structure at 70C (Kim et al., 1989, Protein Eng Des Sel, 2(8), 571-575), and in general higher temperatures expose weaknesses of MNEI and MNEI-based variants, such as:
1. Loops of the beta, and especially the loop between strands 2 & 3 of the beta, do not have a defined secondary structure, and their movement reduces stability (Spadaccini et al., 2001, J Mol Biol., 305(3), 505-514).
2. Backbone of the beta which is exposed to water. Simulation analyses of MNEI at high temperatures show that the main weak point of MNEI is the edge of the interface and backbone between strands 3 & 4. It was found herein that the backbone atom are exposed to water. The simulation also shows that water enter and interference with an hydrogen bond in that area. (Can probably produce a figure for this point).

As described herein, the modified protein described herein has an improved food-related property. The protein's sweetness profile, such as sweetness potency (sugar-like flavor), lack of off-taste, reduced onset time, and reduced lingering taste of the modified protein, may be determined by any known taste test known in the art. For example, a comparison to the sweetness of sucrose or other sweeteners can be made by a taste panel, and the sweetness potency may be graded as detailed in the examples below.

The comparison may be made by determining the modified protein's threshold as compared to a known sweetener, such as sucrose, for example, by determining the minimum concentration required to evoke the sensation of sweetness, or a sweetness profile assessment, including characteristics such as sweetness profile, sweetness onset time, lingering taste, mouthfeel, aftertaste, off-taste, and masking of unwanted tastes.

As used herein, the term sweetening-affecting properties encompass a sweet sensation determined by at least one of a sweetness threshold of about 0.28 mg/L, of about 0.5 mg/L, or higher, sweetness duration of about between 1 to 20 seconds, at times between 2 to 18 seconds, at times between 2-4 seconds

The modified protein, like the reference protein, binds to the sweet receptor.

In some embodiments, the modified protein has a perceived sweetness threshold that is 300-16,000 higher than sugar on a per weight basis.

The sensory profile includes taste kinetics showing taste intensity over time, i.e., onset duration (time until feeling taste), taste duration, and time of lingering taste (corresponding to a gaussian tail). Additional features include off-taste (e.g., due to binding to other receptors), taste roundness, metallic and other side-tastes, synergy with other ingredients (e.g., masking and enhancing other flavors or unwanted tastes, such as stevia), mouthfeel, astringency, and alike.

In some embodiments, the modified protein is characterized by at least one of the following being equal or improved relative to the reference protein: (1) structural thermal stability, (2) functional thermal stability, (3) pH stability, (4) halo-stability, (5) solubility in water or a partly aqueous milieu (e.g., foods containing fat), or (6) shelf-life stability.

The modified protein described herein is characterized by a sweet taste as well as other taste effects (masking unwanted tastes, less aftertaste, less lingering taste, less off-taste, umami taste, better mouthfeel) that may be used as a sweetener in the preparation of a product for oral delivery.

The modified proteins can be used as a flavor modifying agent or a flavor-enhancing agent.

The modified protein described herein is for use as an oral product. In some embodiments, the product is a food or beverage product, a dietary supplement product, or a medicament. For product preparation, the proteins described herein may be combined with any food-grade additive. The food or beverage product may be provided and used in any solid dry form, including, without being limited thereto, fine powder, lyophilizate, granulate, tablets, etc. In some embodiments, the composition is provided in liquid form, for example, as a solute in water (aqueous solution).

The product comprising the modified proteins may have various applications. This includes, without being limited thereto (each of the following constituting a separate embodiment of the present disclosure), utilization as a sweetener, flavor, enhancer, maskers, and proteins that have flavor characteristics in the food and beverage industry (fruit and vegetable juice and nectars, soft drinks, ready-to-drink beverages, syrups, functional drinks, sports drinks, etc.), in the dairy industry, i.e., dairy products, yogurts, and puddings; in the pharmaceutical industry; the naturopathic industry, the nutraceutical industry (e.g., nutraceutical bar), and other healthcare products (e.g., toothpaste and mouthwash), confectionery, candy and gum industry, vegetables (e.g., ketchup or sauces) or any other application that requires the use of a flavor modifying composition as an excipient or additive.

According to some embodiments, the additional food ingredient is selected from a group consisting of sucrose, fructose, glucose, agave nectar, brown rice syrup, date sugar, honey, maple syrup, molasses, monk fruit, sugar alcohols, rare sugars, steviol glycosides, aspartame, sucralose, acesulfame potassium, and dietary fibers.

In some embodiments, the modified protein has structural thermal stability equal or improved relative to the reference protein.

The term “structural thermal stability” or “thermal stability” as used herein refers to the ability of the modified protein to retain its 3D structure at temperatures above that of the reference protein. The 3D structural stability of a protein can be measured by any method known in the art, such as Circular Dichroism (CD), or thermal shift assays such as Differential Scanning Fluorimetry (DSF) or Differential Scanning Calorimetry (DSC) or titration with protein denaturating agents such as guanidinium chloride. The 3D protein structure may influence protein function. Notably, the shelf-life and thermal stability required for food and beverage products may be related to the structural thermal stability and consists of different measurables, e.g., pasteurization (or heat treatment during preparation of the consumer-packaged good final product) can be applied by different protocols and is related to the heat resistance of retaining the protein structure over a very short time.

In some embodiments, the modified protein has functional thermal stability equal or higher relative to the reference protein. The term “functional thermal stability” as used herein refers to the ability of the modified protein to retain its function after exposure to high temperatures compared with the reference protein.

In some embodiments, the modified protein herein may maintain its sweetness effect at a higher temperature or after exposure to a higher temperature for a limited time. In other words, there is no apparent change in the sweetness or sensory profile after product exposure to a temperature above room temperature, at times, up to 50° C., at times up to 100° C., or even up to 150° C. The protein function, e.g., sweetness, may be measured by sensory tests after the protein is cooled down to a temperature in which it can be tasted.

In some embodiments, the modified protein has pH stability being equal or higher relative to the reference protein. pH stability refers to the long-term stability of the modified protein at a wider pH range relative to the reference protein, namely the modified protein maintains the 3D structure and/or function after exposure of the product to any pH from 3 to 8, at times, at a pH of between 4 to 8. For example, a soda like cola has a pH of 2.3-2.5, at which some of the sweet proteins are unstable and lose functionality immediately or after a time that is shorter than the regular shelf-life of the beverage.

In some embodiments, the modified protein has a solubility higher than the reference MNEI protein. Solubility may be in an aqueous, partly aqueous, or non-aqueous milieu, such as foods containing fat.

In some embodiments, the modified protein has an improved shelf-life relative to the reference protein. Improved shelf-life refers to no sensed change in sweetness (function) or physical deterioration of a product comprising the composition (e.g., color change, phase separation, etc.) after exposure of the product to any temperature up to 150° C., at times, to any temperature between 4° C. to 150°, or 100°.

In some other embodiments, the modified protein is characterized by at least one of the following being equal or improved relative to the reference protein (1) folding kinetics, (2) post-translational modification (e.g., glycosylation or acetylation) pattern of the protein is different than the reference protein.

In some embodiments, the modified protein has folding kinetics equal or higher relative to the reference protein. Namely, the protein folding rate from an unfolded or partially folded structure is faster (as assessed in silico, e.g., by molecular dynamics or by experimental in vitro or in vivo methods). Alternatively, faster folding kinetics refers to slower unfolding kinetics in denaturation experiments, e.g., by denaturant titrations (e.g., guanidinium chloride and/or high-concentration urea) or other methods.

In some embodiments, the modified protein is characterized by an expression yield equal or higher relative to the reference protein in the host organism assessed.

In some embodiments, the modified protein has a pI value of between 8.6 to 9.5.

The modified protein described herein is characterized by a sweet taste as well as other taste effects (masking unwanted tastes, less aftertaste, less lingering taste, less off-taste, less lingering taste onset, and umami taste) may be used as a sweetener in the preparation of a product for oral delivery.

The modified protein can be used as a flavor modifying agent or a flavor-enhancing agent.

The modified protein described herein is for use as an oral product. In some embodiments, the product is a food product, a food supplementary product, or a medicament. For product preparation, the proteins described herein may be combined with any food-grade additive. The food product may be provided and used in any solid dry form, including, without being limited thereto, fine powder, lyophilizate, granulate, tablets, etc. In some embodiments, the composition is provided in liquid form, for example, as a solute in water (aqueous solution).

The product comprising the modified proteins may have various applications. This includes, without being limited thereto (each of the following constituting a separate embodiment of the present disclosure), utilization as a sweetener, flavor, enhancer, or masker in the food and beverages industry (fruit and vegetable juice and nectars, soft drinks, ready-to-drink beverages, syrups, functional drinks, sports drinks, etc.), in the dairy industry, i.e., dairy products, yogurts, and puddings, in the pharmaceutical industry, in the naturopathic industry, nutraceutical industry, and other healthcare products (e.g., toothpaste and mouthwash), confectionary, candy and gum industry, vegetables (e.g. ketchup or sauces) or any other application that requires the use of a flavor modifying composition as an excipient or additive.

The product may comprise additional food ingredients. In some embodiments, the food ingredient is a sweetener, for example, a steviol glycoside. The combination of the modified protein described herein and a steviol glycoside produce a synergetic effect. Thus, in some embodiments, the product comprises at least one modified protein as shown in Table 10 and a steviol glycoside.

Wherein Stevia (denoted herein a steviol glycoside or mixture thereof) and/or its varieties are combined with the modified protein of the present invention at the range of 0.5° Bx to 8° Bx sucrose equivalent, the modified protein represents the replacement of 30% to 70% of Sucrose sweetness. The perceived sweetness intensity is at least 100% of stevia solution at a sucrose equivalent of 0.5° Bx to 8° Bx. The perceived lingering sensory profile is superior to 100% of a stevia solution at a sucrose equivalent of 0.5° Bx to 8° Bx. The perceived sourness sensory profile is superior to 100% of a stevia solution at a sucrose equivalent of 0.5° Bx to 8° Bx.

According to some embodiments, the additional food ingredient is selected from the group consisting of sucrose, agave nectar, brown rice syrup, date sugar, honey, maple syrup, molasses, monk fruit, sugar alcohols, rare sugars, aspartame, sucralose, acesulfame potassium, and dietary fibers.

In some embodiments, the formulations described herein provide a sugar-like taste profile with a decreased, eliminated, or masked aftertaste or off-flavor (e.g., metallic or licorice taste) or a decreased, eliminated, or masked bitterness or decreased, eliminated, or masked sweet taste lingering.

It should be noted that the modified proteins, according to the invention, can be produced by any method known in the art, for example, the protein can be produced synthetically, by recombinant DNA technology, or by protein production in microorganisms via fermenters, plants, plant callus, or other bioreactors. In some embodiments, the modified proteins may be produced in bacteria, such as E. Coli. In some other embodiments, the modified proteins may be produced yeast, such as Saccharomyces cerevisiae or Pichia pastoris. In some other embodiments, the modified proteins may be produced in filamentous fungi such as Trichoderma, or Aspergillus.

The term “yeast and filamentous fungi” include, but are not limited to any Kluyveromyces sp., such as Kluyveromyces lactis, Kluyveromyces marxianus, Saccharomyces sp., such as Saccharomyces cerevisiae, Pichia sp., such as Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pyperi, Pichia stiptis, Pichia methanolica, Hansenula polymorpha, Candida albicans, any Aspergillus sp., such as Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens, Myceliopthora and Neurospora crassa.

In some embodiments, the DNA sequence of the chosen amino acid sequence is optimized at the RNA and DNA levels. At the RNA level, this includes minimization of RNA secondary structures to ensure quick insertion into the ribosome. At the DNA level, this includes codon optimization to the host organism (taking into account the RNA-level optimization). Codon-usage optimization provides preference for using the most abundant tRNA in the host organism for each amino acid expressed.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. In some embodiments, the term “about” refers to ±10%.

It should be noted that various embodiments of this invention may be presented in a range format. The description of a range should be considered to have specifically disclosed all the possible sub ranges as well as individual numerical values within that range. For example, description of a range such as from 1 to 6 or between 1 and 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6.

As used herein, the forms “a”, “an” and “the” include singular as well as plural references unless the context clearly dictates otherwise.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments unless the embodiment is inoperative without those elements.

It should be noted that the various embodiments and examples detailed herein in connection with various aspects of the invention may be applicable to one or more aspects disclosed herein. It should be further noted that any embodiment described herein, for example, related to method, may be applied separately or in various combinations. Various embodiments and aspects of the present invention as delineated hereinabove and as claimed in the claims section below find experimental support in the following examples. The phrases “in another embodiment” or any refence made to embodiment as used herein do not necessarily refer to different embodiment, although it may. Thus, various embodiments of the invention can be combined (from the same or from different aspects) without departing from the scope of the invention.

Various embodiments and aspects of the present invention as delineated herein above and as claimed in the claims section below find experimental support in the following examples.

Disclosed and described, it is to be understood that this invention is not limited to the particular examples, methods steps, and reactors disclosed herein as such methods steps and reactors may vary somewhat. It is also to be understood that the terminology used herein is used for the purpose of describing particular embodiments only and not intended to be limiting since the scope of the present invention will be limited only by the appended claims and equivalents thereof.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### NON-LIMITING EXAMPLES

### Example 1: Computational Design of Novel Proteins

Design of polypeptides (proteins) was done by using the amino acid sequence of MNEI as a reference protein for modifications. Single chain monellin, MNEI (denoted herein as SEQ ID NO:8), is a polypeptide composed of 96 amino acids, with a molecular weight of ˜11 kD and pI ˜8.7.

### Methods

Molecular dynamics (MD)—system setup and simulations:

For all simulated proteins residue protonation states were set as appropriate at pH 7.0 using PROPKA (Olsson et al., 2011, J Chem Theory Comput, 7(22), 525-537) version 3.4. MD simulations were performed using the GROMACS software (Abraham et al., 2015, SoftwareX(1-2), 19-25) version 2022.1. Systems were parameterized using the Amber99SB all-atom force field (Hornak et al., 2006, Proteins, 65, 712-725). Independent replicates of each system were conducted for 50 ns each at 433K.

Molecular dynamics (MD)—measures:
1. Native Contacts were calculated using Best-Hummer fraction of native contacts function (Best et al., 2013, PNAS, 110(44), 17874-17879) as implemented in MDTraj version 1.9.6 (McGibbon et al., 2015, Biophys J., 109(8), 1528-32). A native contact is a contact between two amino acids that are not neighboring in the amino acid sequence but are spatially close in the protein's native state tertiary structure. These contacts are used in molecular dynamics to measure deviation. A Beta constant of 50 1/nm, a Lambda constant of 1.8, and a native contact cutoff of 0.45 nm were used. The fraction of native contacts was averaged over the simulation time course. More contacts between heavy atoms of the protein means higher stability during simulations.
2. Helix H-bonds during simulation were calculated using GROMACS hbond utility using intra-bacbone-mediated hydrogen bonds, in order to detect secondary structure unfolding. The helix residues are defined as residues 10-26.
3. Beta-Sheet H-bonds during simulation were calculated using GROMACS hbond utility using intra-backbone-mediated hydrogen bonds. Beta-sheet residues are typically defined as residues 2-6, 35-37, 42-48, 54-66, 69-78, 82-90 numbered according to the sequence and structure of MNEI (SEQ ID NO:8, 2O9U).
4. Average H-bonds helix—measures the average number of intra-helical backbone-mediated H-bonds for the helix. This value shows the stability of the helix during simulation. A higher value represents a more canonical (and likely favorable) helix.
5. Average H-bonds beta—measures the average number of inter-strand-mediated H-bonds for the beta sheet. This value shows the stability of the beta sheet during simulation. A higher value is expected for more stable beta-sheets in turn leading to a more stable protein.
6. Water-beta-sheet backbone H-bonds—measures the average number of water molecules around the beta sheet of the protein, that are sufficiently stable to enable a bond to the protein's backbone in segments composed of beta-strand secondary structures.
7. Fraction of canonical helix—this is the fraction of H-bonds in the helix that are of type i→i+4. There are three types of helical H-bonds: i:i+3, i:i+4 and i:i+5, indicating a 3-10 helix, alpha-helix and pi-helix, respectively. The alpha-helix with i:i+4 bonds is the most canonical. This value indicates that an alpha-helix is maintained during simulation. The higher the fraction of canonical H-bonds the more stable the helix is expected to be.
8. Sum RMSD averages of RMSD of proteins' C-alpha atoms in the helix region (residues 10-26), loops (residues 38-42, 49-53, 67-68, 79-81, 7-9, 27-34), C-terminus of the protein (residues 90-96) and beta-sheet (residues 2-6, 35-37, 42-48, 54-66, 69-78, 82-90) were calculated using GROMACS rms utility. Averages were then summed to produce a single measure. The residues are numbered according to the sequence and structure of MNEI (SEQ ID NO:8, PDB ID: 2O9U).
9. RMSF (root mean square fluctuations) of proteins' backbone atoms throughout the MD simulations. These were calculated using GROMACS rmsf utility and averaged for each residue.

Molecular Graphics: Structural analyses, measurements, Figures and movie clips were performed with either VMD (Humphrey, W., Dalke, A. and Schulten, K, VMD—Visual Molecular Dynamics, J. Molec. Graphics 1996, 14.1, 33-38.) version 1.9.4. Figures and movies were rendered using VMD or PyMol (The PyMOL Molecular Graphics System, Version 2.5.2, Schrödinger, LLC.).

### Structure Prediction and Energy Calculations:

New variants were modeled by using the template of MNEI or DM31 crystal structures (MNEI based on a structure from the Protein Data Bank (PDB), ID 2O9U; DM31 as determined by the Weizmann Institute of Science Structural Proteomics Unit).

The resulting structures were submitted to the Rosetta software, for further energy minimization and calculation of energy scores, using Rosetta 3.8 (Schueler-Furman et al., 2005, Science, 310, 638-642; Baker, 2006, Philos Trans R Soc Lond B Biol Sci., 361, 459-63; Kaufmann et al., 2010, Biochemistry, 49, 2987-2998). The minimization and scoring were performed using Rosetta's FastRelax protocol and the REF2015 energy function (Park et al., 2016, J Chem Theory Comput, 12, 6201-6212). For each input sequence, the protocol was repeated for at least 30K times, thus obtaining multiple structures along with their Rosetta Energy Function (REU) scores. For each variant, the 30K scores were sorted, and the 500 lowest scoring structures were used for further examination and analyses. For MNEI and DM31, the input structures were the corresponding crystal structures (2O9U for MNEI), and the protocol was repeated 100K times. Table 1 presents new variants selected for expression in the lab, along with their corresponding Rosetta Energy Units (REU) scores. For some analyses, the lowest energy structure for each new variant was used.

### Additional Protein Features:

Visualization and analysis of hydrogen bonds was done with PyMol. In addition, hydrogen bonds were examined using the Baker-Hubbard method (Baker and Hubbard, 1984, Progress in Biophysics and Molecular Biology, 44.2, 97-179), as implemented in the MDTraj Python package (McGibbon et al., 2015, Biophys J., 109(8), 1528-1532) version 1.9.6.

An additional measure of hydrophobicity was used—SAP (Spatial Aggregation Propensity) score (Lauer et al., 2012, J Pharm Sci, 101(1), 102-115), calculated using Rosetta. This score measures local hydrophobicity of surface patches. Such surface regions can potentially form hydrophobic interactions with other patches on other proteins—thus increasing the risk of aggregation. The higher the score the greater the aggregation propensity.

An additional measure was used for estimating protein structure packing—VoroMQA (Olechnovič & Venclovas, 2017, Proteins, 85, 1131-1145. this method combines statistical potentials with the use of interatomic contact areas instead of distances. Contact areas, derived using Voronoi tessellation of protein structure, are used to describe and integrate both explicit interactions between protein atoms and implicit interactions of protein atoms with solvent. VoroMQA produces scores at atomic, residue, and global levels, all in the fixed range from 0 to 1.

### Results

Table 1 presents the calculated Rosetta Energy Unit (REU) scores for new proteins variants selected based on the modeling.

For both the minimization and calculation, Rosetta's REF2015 energy function was used. The table also presents the SAP score, referring to hydrophobicity and indicating aggregation propensity. Residue numbers are in accordance to MNEI as set forth in SEQ ID NO:8, ΔE50/ΔF52/ΔR53 indicated deletion of the E50, F52 and R53.

TABLE 1
Rosetta REU and SAP scores for the novel DM variants.

| Protein | Energy (REU) | SAP |
|---|---|---|
| MNEI | −315.345 | 67.64 |
| DM31 | −321.66 | 58.89 |
| DM42 | −320.746 | 55.03 |
| DM43 | −319.259 | 57.71 |
| DM47 | −322.629 | 57.73 |
| DM46 | −320.277 | 62.48 |
| DM70 | −325.41 | 63.12 |
| DM72 | −322.539 | 57.35 |
| DM41 | −319.98 | 53.31 |
| DM57 | −320.61 | 54.50 |
| DM65 | −320.54 | 62.55 |
| DM66 | −319.96 | 54.86 |
| DM68 | −316.787 | 55.32 |
| DM69 | −318.194 | 55.85 |
| DM73 | −321.03 | 53.41 |
| DM75 | −321.43 | 54.93 |
| DM77 | −320.38 | 54.49 |
| DM84 | −321.05 | 55.86 |
| DM85 | −322.19 | 55.28 |
| DM87 | −321.47 | 58.47 |
| DM91 | −322.77 | 54.74 |
| DM92 | −319.52 | 55.46 |
| DM93 | −324.93 | 54.34 |
| DM96 | −321.55 | 56.49 |
| DM103 | −320.09 | 54.73 |
| DM104 | −320.13 | 54.34 |
| DM115 | −323.89 | 62.00 |
| DM116 | −324.13 | 61.36 |
| DM131 | −320.83 | 58.43 |
| DM132 | −322.81 | 56.70 |
| DM144 | −323.48 | 53.64 |
| DM145 | −320.94 | 54.72 |
| DM150 | −322.91 | 53.79 |
| DM151 | −323.04 | 53.75 |
| DM94 | −322.53 | 55.90 |
| DM97 | −325.954 | 57.47 |
| DM99 | −320.207 | 55.05 |
| DM143 | −323.496 | 54.88 |
| DM152 | −320.611 | 56.07 |
| DM108 | −314.802 | 55.36 |
| DM117 | −322.166 | 55.63 |
| DM330 | −319.547 | 56.00 |
| DM464 | −318.944 | 53.72 |
| DM508 | −321.948 | 56.91 |
| DM509 | −318.617 | 54.39 |
| DM506 | −321.492 | 55.24 |
| DM491 | −324.735 | 56.95 |
| DM498 | −318.67 | 53.64 |
| DM420 | −323.529 | 55.67 |
| DM424 | −322.575 | 59.83 |
| DM452 | −319.446 | 55.72 |

Table 2 presents a summary of the results obtained from molecular simulations of the novel proteins. Average native contacts were calculated using fraction of native contacts. Average RMSD was calculated using protein's backbone atoms. Average H-bonds in the helix and beta-sheet backbone were calculated in GROMACS (see Molecular dynamics, under Methods), Average RMSD of specific secondary structure elements Loopwere calculated using protein's C-alpha atoms. Two RMSD results are mentioned here. See Methods for elaborated description.

TABLE 2
Molecular Dynamics (MD) simulations' results

| | | | | | | Water- |
|---|---|---|---|---|---|---|
| | | | | | | Beta |
| | Average | Average | Average | | Fraction of | sheet |
| | Native | Hbonds | Hbonds | Sum | Canonical | backbone |
| DM | contacts | helix | beta | RMSD | Helix | Hbonds |
| MNEI | 862.538 | 4.995 | 26.722 | 1.432 | 0.56 | 27.234 |
| DM41 | 987.085 | 9.525 | 28.189 | 0.454 | 0.824 | 25.918 |
| DM57 | 1083.516 | 9.135 | 28.224 | 0.452 | 0.791 | 25.863 |
| DM65 | 1035.571 | 9.136 | 28.294 | 0.579 | 0.794 | 25.974 |
| DM66 | 1002.831 | 9.285 | 28.22 | 0.442 | 0.799 | 25.924 |
| DM68 | 975.362 | 9.536 | 28.037 | 0.45 | 0.791 | 26.065 |
| DM69 | 1006.432 | 9.504 | 28.217 | 0.59 | 0.789 | 25.814 |
| DM73 | 937.772 | 9.269 | 28.223 | 0.439 | 0.805 | 26.054 |
| DM75 | 981.648 | 9.321 | 28.074 | 0.427 | 0.806 | 25.756 |
| DM104 | 980.764 | 9.319 | 28.119 | 0.469 | 0.808 | 25.956 |
| DM99 | 1006.706 | 9.114 | 28.173 | 0.539 | 0.796 | 25.848 |
| DM143 | 929.667 | 8.933 | 28.111 | 0.500 | 0.79 | 25.938 |
| DM152 | 976.897 | 8.783 | 28.064 | | 0.759 | 26.329 |
| DM92 | 1009.415 | 9.456 | 28.334 | 0.367 | 0.829 | 25.750 |
| DM464 | 1069.732 | 9.402 | 28.089 | 0.521 | 0.788 | 25.872 |
| DM330 | 969.059 | 9.284 | 28.216 | 0.548 | 0.802 | 25.879 |
| DM116 | 1038.806 | 9.342 | 28.252 | 0.4765 | 0.81 | 25.743 |
| DM144 | 1041.763 | 9.444 | 28.136 | 0.4508 | 0.808 | 25.772 |
| DM145 | 994.553 | 8.838 | 28.147 | 0.536 | 0.759 | 25.99 |
| DM115 | 1025.408 | 9.286 | 28.219 | 0.4762 | 0.8 | 25.475 |
| DM108 | 899.533 | 9.276 | 28.08 | 0.443 | 0.803 | 25.821 |
| DM61 | 989.007 | 9.252 | 28.245 | 0.444 | 0.798 | 25.620 |
| DM97 | 1013.577 | 8.973 | 27.924 | 0.553 | 0.796 | 26.477 |
| DM93 | 912.769 | 8.922 | 27.929 | 0.623 | 0.771 | 26.299 |
| DM341 | 886.399 | 9.202 | 27.816 | 0.553 | 0.799 | 26.606 |
| DM94 | 1034.045 | 9.336 | 28.088 | 0.474 | 0.801 | 25.944 |
| DM72 | 984.172 | 9.179 | 28.277 | 0.504 | 0.792 | 25.96 |
| DM77 | 933.122 | 8.689 | 28.088 | 0.501 | 0.768 | 26.170 |
| DM491 | 918.148 | 8.884 | 27.899 | 0.553 | 0.769 | 26.624 |
| DM117 | 1013.185 | 8.952 | 28.019 | 0.567 | 0.789 | 26.040 |
| DM84 | 919.735 | 9.11 | 28.153 | 0.632 | 0.787 | 25.943 |
| DM96 | 972.287 | 9.246 | 27.942 | 0.493 | 0.788 | 26.294 |
| DM506 | 996.314 | 9.148 | 28.158 | 0.561 | 0.801 | 25.887 |
| DM87 | 1011.401 | 8.225 | 27.901 | 0.619 | 0.74 | 26.253 |
| DM508 | 1044.928 | 9.011 | 28.157 | 0.461 | 0.79 | 25.997 |
| DM509 | 1038.119 | 9.326 | 28.134 | 0.524 | 0.758 | 25.881 |
| DM46 | 984.914 | 9.195 | 28.158 | 0.447 | 0.808 | 25.530 |
| DM150 | 997.109 | 9.04 | 28.246 | 0.521 | 0.795 | 26.299 |
| DM151 | 1038.136 | 9.151 | 28.122 | 0.492 | 0.802 | 26.380 |
| DM31 | 975.971 | 9.145 | 28.115 | 0.471 | 0.797 | 25.958 |
| DM498 | 1033.049 | 9.237 | 28.031 | 0.528 | 0.816 | 25.781 |
| DM85 | 1033.743 | 9.168 | 27.922 | 0.477 | 0.778 | 26.186 |
| DM91 | 1016.008 | 9.097 | 28.082 | 0.675 | 0.797 | 25.964 |
| DM103 | 1009.907 | 8.814 | 28.171 | 0.521278 | 0.78 | 25.968 |
| DM432 | 1052.854 | 9.343 | 28.251 | 0.455208 | 0.813 | 26.454 |
| DM489 | 884.06 | 8.323 | 26.963 | 0.747473 | 0.737 | 27.379 |
| DM505 | 921.519 | 8.45 | 27.898 | 0.544583 | 0.727 | 26.169 |
| DM131 | 831.402 | 7.676 | 27.334 | 0.770456 | 0.682 | 26.958 |
| DM132 | 901.696 | 9.3 | 28.375 | 0.495914 | 0.803 | 25.812 |
| DM510 | 874.321 | 8.762 | 27.98 | 0.593 | 0.718 | 26.282 |
| DM452 | 980.368 | 9.262 | 27.853 | 0.605 | 0.796 | 26.352 |
| DM70 | 966.97 | 9.264 | 28.216 | 0.458 | 0.799 | 26.108 |
| DM47 | 943.741 | 9.177 | 28.225 | 0.550 | 0.8 | 25.977 |
| DM420 | 997.634 | 9.165 | 27.784 | 0.507 | 0.794 | 26.176 |
| DM424 | 982.653 | 9.176 | 28.009 | 0.464 | 0.786 | 26.069 |

### Md Consideration for New Modified Proteins (Variants)

As noted herein, the objective of the MD analysis was to evaluate, among others, different regions in the protein (MNEI) and variants thereof in order to predict novel proteins with improved properties. Modification was evaluated, for example, in alpha helix region, beta sheet region and in the core region.

### Example 1A: Helix Capping Variants

To identify the targets for design, molecular dynamics was done on MNEI simulation on high temperature conditions. Lα2 was identified as a target region for stabilization by molecular dynamics. Thus, the objective was to identify modified proteins that would stabilize the loop between the helix and the 2^{nd} beta strand.

Table 3 presents computation data analyses of novel variants which are considered as helix capping variants. AUC (Area Under Curve) differences (ΔAUC) were calculated using the average RMSF of three MD simulation repeats for each DM, as ΔAUC=(AUC(variant)-AUC(DM31)).

TABLE 3
Computational analyses for novel MNEI helix stabilizing variants

| DM | ΔAUC |
|---|---|
| DM42 | −1.374 |
| DM43 | 0.589 |
| DM65 | −0.878 |
| DM69 | −1.344 |
| DM92 | −2.816 |
| DM464 | 0.553 |
| DM330 | −1.454 |
| DM66 | −1.642 |
| DM508 | −0.485 |
| DM509 | 1.405 |
| DM73 | 1.439 |
| DM116 | −0.684 |
| DM144 | 1.574 |
| DM145 | 2.174 |
| DM115 | −1.565 |
| DM510 | 3.22 |
| DM41 | −1.189 |

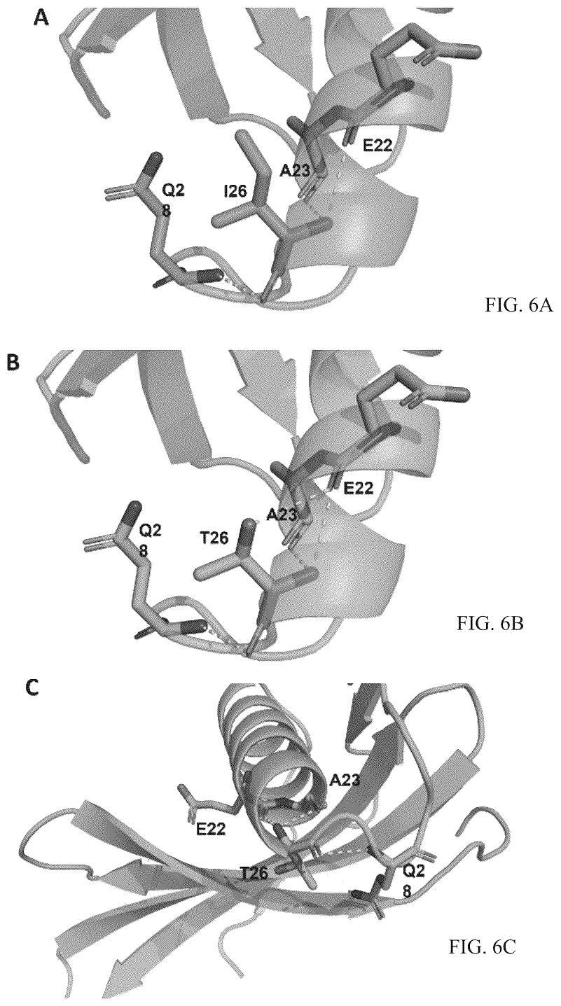

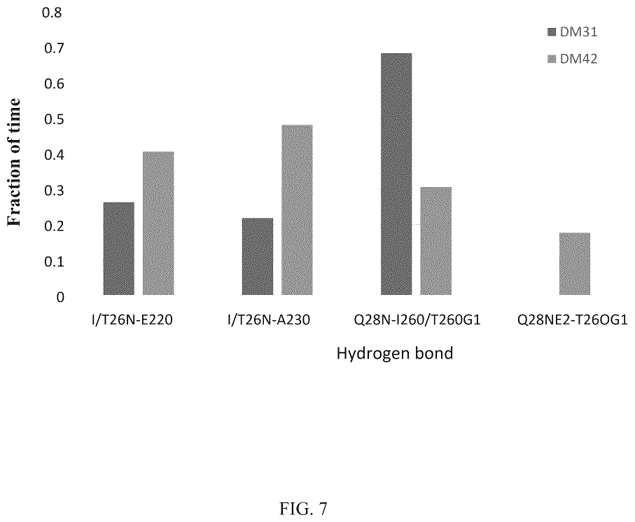

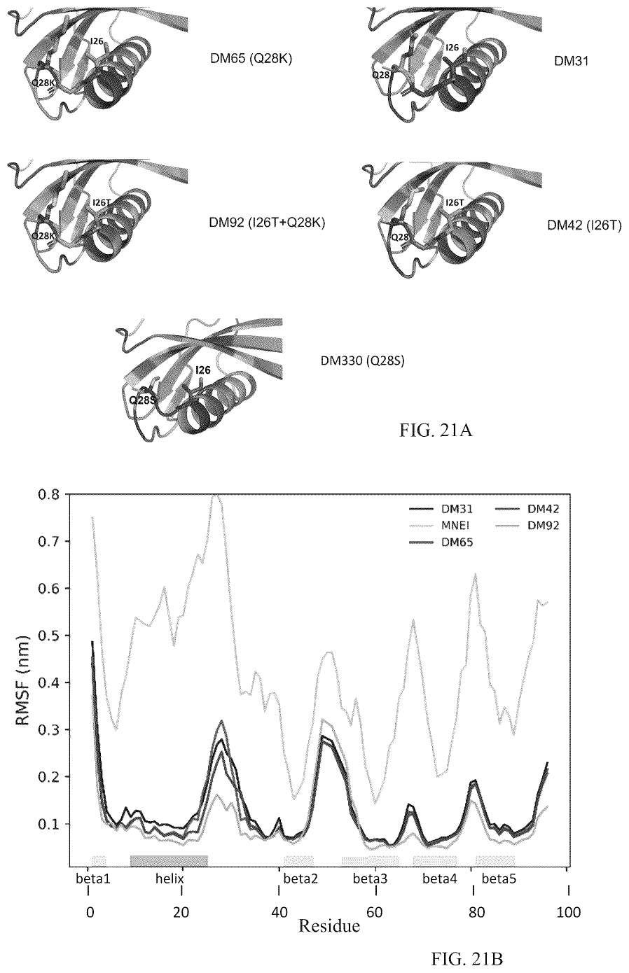

DM42 (I26T): predicted to be favorable based on a helix capping analysis (using CAPS-DB database, Segura et al., 2012, Nucleic Acids Res. 40(Database issue)). The substitution removes a hydrophobic residue from the protein's surface, thus lowering the SAP score (Table 1). MD simulations have shown good stability and that hydrogen bonds via backbone between 26 and E22, and between position 26 and A23, were more stable in DM42 compared to DM31 (FIG. 21B, Table 2 & FIG. 7). Hydrogen bonds can be seen in the models (FIG. 6). VoroMQA analysis demonstrates more favorable packing of residue 26 and its surroundings (FIG. 21A). Furthermore, MD analyses show indications to increased stability (Table 2). Experimental DSF analysis on DM42 measured a very high melting temperature (99° C.) (Table 7A).

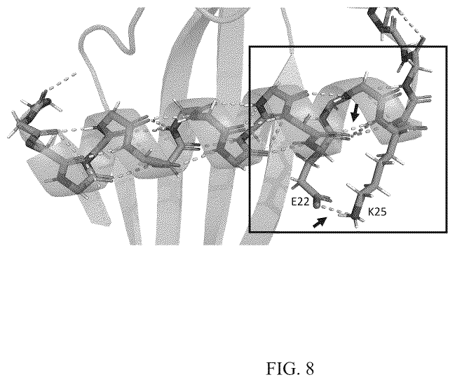

DM43 (K25R): The SAP score for this DM indicates decreased hydrophobicity (Table 1). Native contacts and backbone-mediated H-bonds inside the beta-sheet and inside the helix of DM43 are more stable during simulations in 433K compared to MNEI (Table 2), suggesting increased stability of this variant. The side-chain of K25 displays a hydrogen bond with the side-chain of E21 in DM31 (FIG. 8). Simulations show that in DM31 K25 attack the E22 backbone, reducing the helix stability, while in DM43, R25 keeps the R25-E21 residue-residue Hbond. Substitution K25R is expected to increase the bond's strength with more positive charge. The hydrogen bonds between K25 and E22 contribute to the stability of the helix (FIG. 8, marked by arrows), and are further stabilized by the substitution in DM43, namely, K25R. Experimental DSF analysis on DM43 measured a high melting temperature (94° C.), which was reflected increased ability to maintain sweet in spit of extreme temperatures (Table 7A).

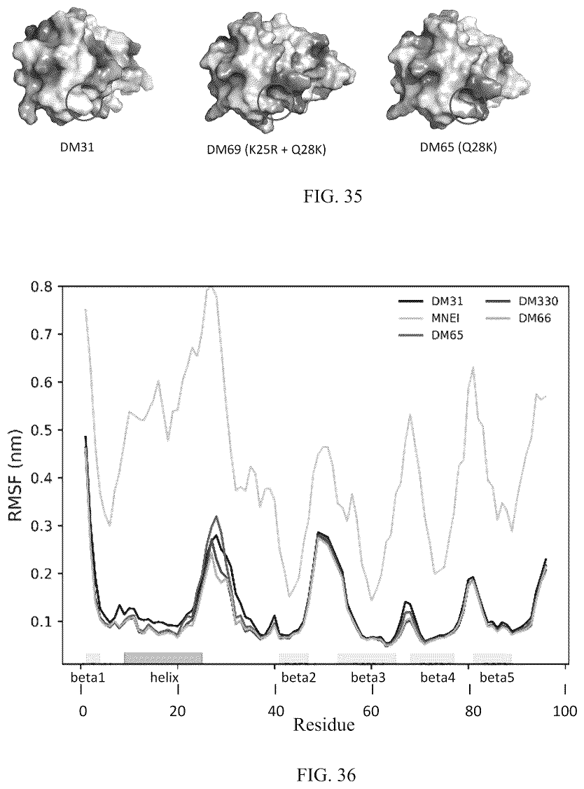

DM69 (K25R+Q28K): a combination of DM43 (K25R) and DM65 (Q28K). The SAP score for this DM is relatively low because of these highly polar substitutions (Table 1). Molecular dynamics simulations and RMSF for this DM determined it is favorable (Table 3 & FIG. 34). DM69 is expected to be sweeter than DM31, in light of the electrostatics changes presented in FIG. 35. Experimental DSF analysis on DM69 measured a very high melting temperature (97.5° C.) (Table 7A).

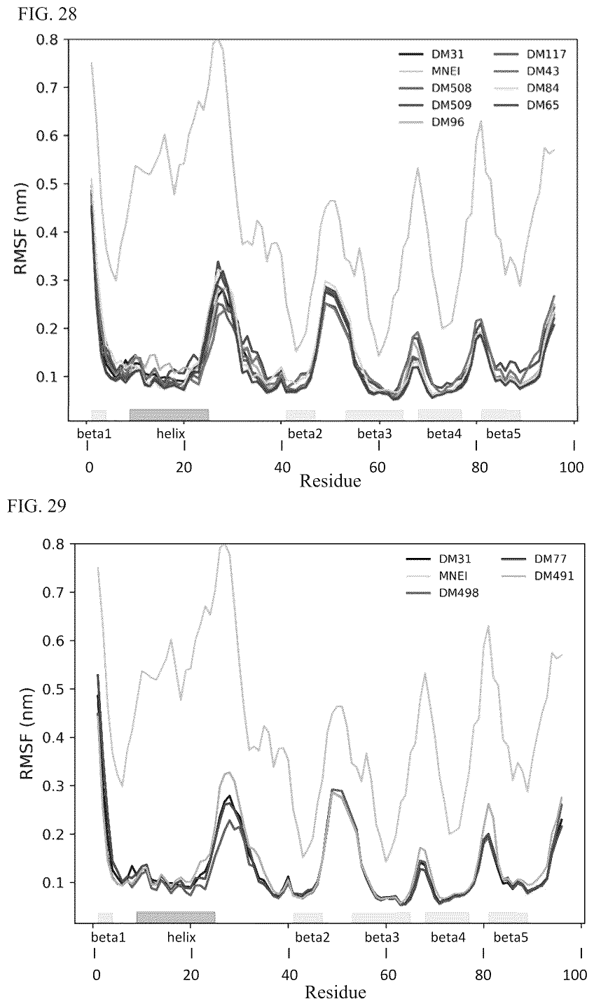

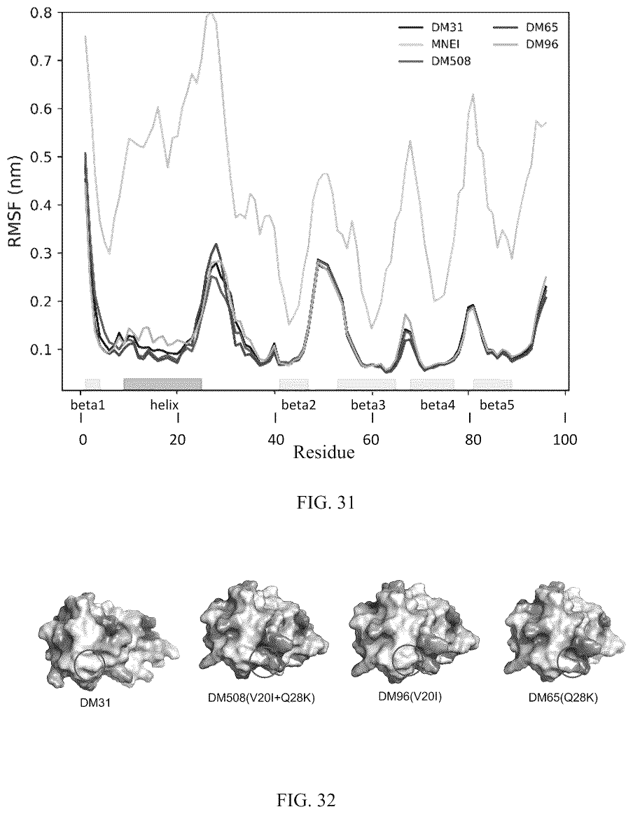

DM508 (V20I+Q28K): combines DM96 (V20I) which is core oriented and DM65 (Q28K), both having effect on the protein's helix. As evident by molecular dynamics simulations, RMSF indicates good stability, also specifically in the Lα2 loop (FIG. 28 & FIG. 31). This manifested in increased ability to maintain sweetness in high temperature conditions (Table 7A). According to the SAP score, DM508 is expected to possess reduced hydrophobicity (Table 1), as can be also seen in APBS (FIG. 32).

DM509 (K25R+V20I): combines substitutions of DM96 (V20I) which is core oriented and DM43 (K25R)— both having effect on the protein's helix. As evident by molecular dynamics simulations, the resulting RMSF indicates improved stability (FIG. 28 & Table 3). This DM has a low SAP score, indicating reduced hydrophobicity (Table 1).

DM92 (I26T+Q28K): The Lα2 region was further stabilized by substituting position 28 with a lysine residue (Q28K). MD simulations show that the wild type Gln does not display a stable interaction. The substitution to lysine at this position allows for a hydrophobic interaction with the region around position 28 and thus the region remains fixed with improved rigidity. This is possible in conjunction with I26T which brings the surrounding backbone closer to the protein. The combination of the substitutions improves the packing of both residues in comparison to their packing when present alone (FIG. 21A & 21B). In addition, this combination reduces the SAP score of for this DM (Table 1), most likely due to the replacement of a hydrophobic residue with a polar residue. RMSF indicates good stability (Table 3, FIG. 21B).

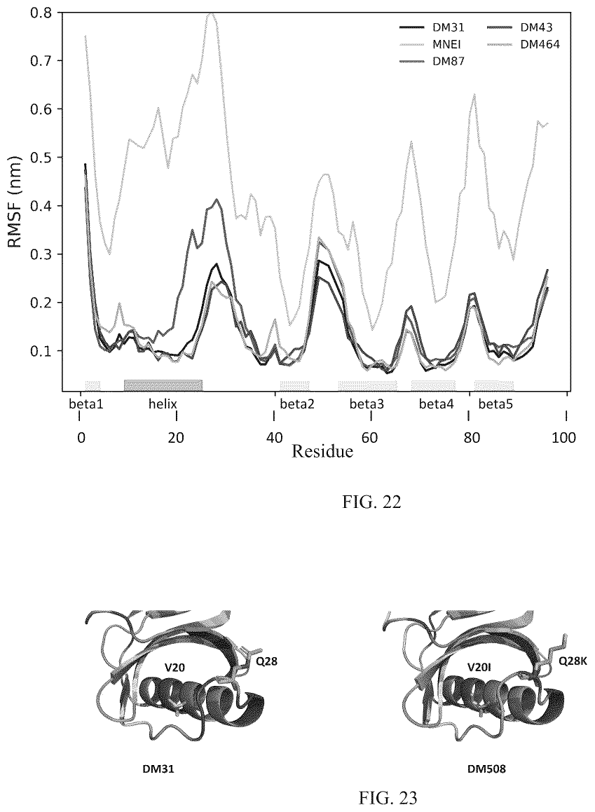

DM464 (K25R+D68N): The combination of K25R+D68N is expected to contribute to both stability and sweetness reducing the negative charge and increasing the positive local surface charge (D68N). The substitutions also reduce the hydrophobicity of the protein as evidenced by the very low SAP score (Table 1). RMSF plots demonstrates good stability (FIG. 22).

FIG. 22 Average RMSF (Root Mean Square Fluctuations) of DM31, MNEI, DM87 (D68N), DM43 (K25R), DM464 (K25R+D68N). Each variant is depicted by a different color. When comparing the RMSF of the helix end region (25-30aa) of the different DM to DM31 (black line), DM43, DM464 demonstrate lower RMSF.

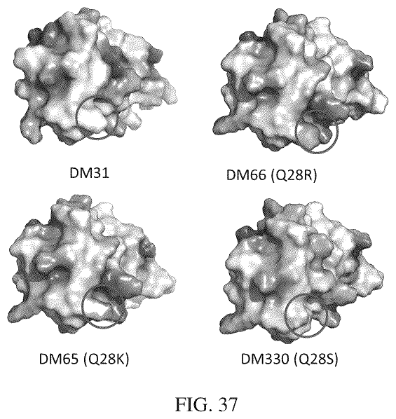

DM330 (Q28S): DM330 was designed to have improved packing. VoroMQA analysis showed a favorable packing (FIG. 21A), and this variant demonstrates good stability in RMSF and MD (Table 2 & 3, FIG. 36). DM330 is expected to be sweeter than DM31, based on electrostatics analysis (FIG. 37).

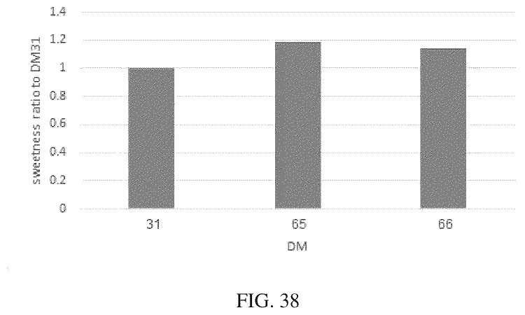

DM66 (Q28R): Molecular dynamics suggests good stability (Table 2 & 3, FIG. 36). DM66 was predicted to be sweeter than DM31 based on electrostatics analysis (FIG. 37), and proven to be extremely sweet by a sensory panel with high ability to keep sweetness in high temperatures (Table 7A & FIG. 38). Experimental DSF analysis on DM66 measured a high melting temperature (95° C.) (Table 7A). In addition to these improved properties, the SAP score for DM66 is low (Table 1).

DM65 (Q28K): A sensory panel proved this variant to have very high sweetness, and high sweetness durability to high temperature as reflected in heat treatment (Table 7A & FIG. 38). Since the modification is approximate to position 26 we have tested the its effect on packing, showing improved packing (FIG. 21A). This linkage of sweetness to Monellin's packing was never suggested before. Experimental DSF analysis on DM65 measured a high melting temperature of (93° C.) (Table 7A). DM65 shows good stability according to MD simulations in high temperature, as demonstrated also by RMSF plot (Table 3 & FIG. 21B, FIG. 34).

DM41 (I26S): predicted to be favorable based on a helix capping analysis (using CAPS-DB database, Segura et al., 2012, Nucleic Acids Res. 40(Database issue)). The substitution removes a hydrophobic residue from the protein's surface, thus lowering the SAP score (Table 1). In addition, AUC indicates good overall stability (Table 3). Experimental DSF analysis on DM41 measured high melting temperature (94° C.) (Table 7A).

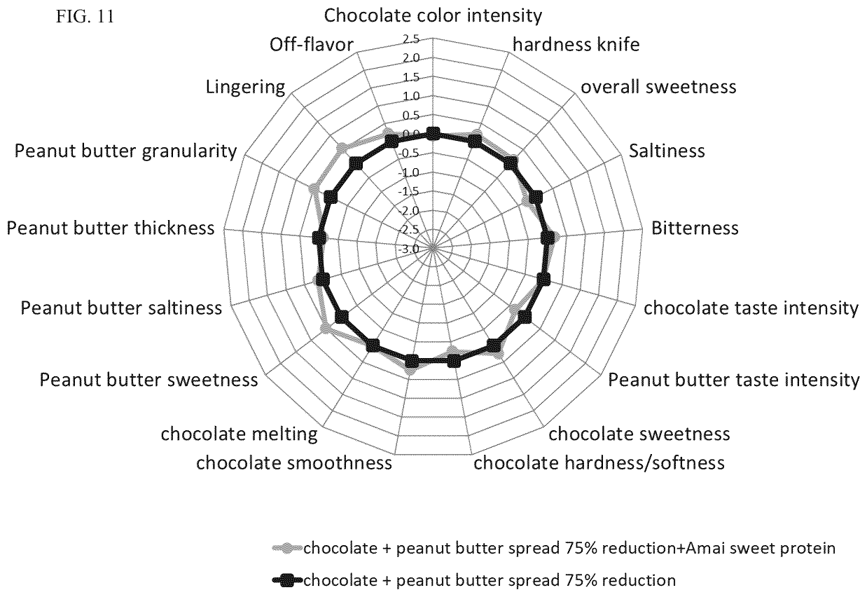

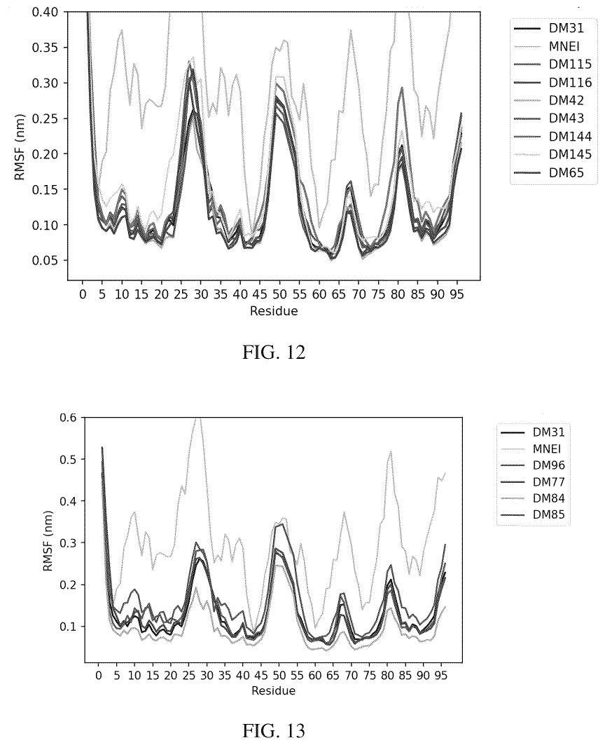

FIG. 12 shows that DM65, DM42 and DM43 have good stability compared to MNEI. DM145, DM144, DM115, DM116 are more stable than MNEI. Native contacts of DM42, DM43, DM115, DM116, DM144 are increased in comparison to MNEI (Table 2). DM42, DM43 and DM144 display an increased number of intra-helical backbone-mediated H-bonds in comparison to MNEI (Table 2). DM65, DM145, DM115 and DM116 display more intra-helical backbone-mediated H-bonds than in MNEI (Table 2). DM42 helix is on average more canonical during MD simulations (Table 2). As demonstrated by MD simulations, the helix of DM43, DM115, DM116, DM144, DM145 and DM65 is, on average, more canonical than that of MNEI (Table 2). DM115 and DM43 have in addition low ΔAUC of RMSF, and DM144, DM145, and DM330 have low SAP scores (Table 1 & Table 2).

FIG. 38 shows the relative sweetness of DM65 and DM66 compared to DM31 in 10 Brix. Both proteins show increased sweetness by 15% and 19%, respectively, according to a sensory panel.

### Example 1B: Sweetness Variants

TABLE 4
Computational analyses for novel sweetness related MNEI variants

| DM | Substitutions | ΔAUC |
|---|---|---|
| DM87 | D68N | 2.934 |
| DM108 | D68T | 2.159 |
| DM61 | D68S | 2.143 |
| DM57 | T33R | −0.853 |
| DM97 | R31T + R84L | 3.884 |
| DM93 | D68N + R84L | 31.389 |
| DM47 | R31T | 9.7955 |
| DM341 | K36T | 0.139 |
| DM94 | D68N + R31T | −0.629 |
| DM72 | N35T | −1.092 |
| DM506 | V20I + D68N | −0.6571 |
| DM65 | Q28K | −0.878 |
| DM66 | Q28R | −1.642 |
| DM69 | Q28K+ | −1.344 |

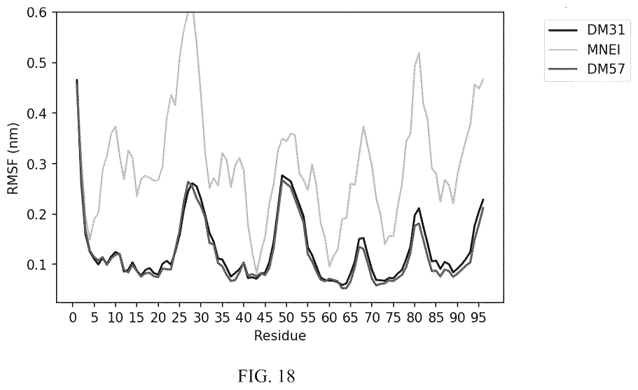

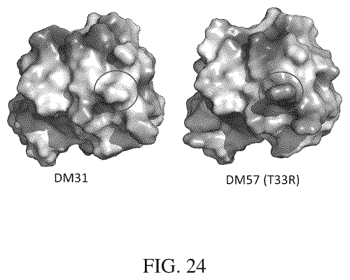

DM57 (T33R): APBS analysis (FIG. 24) shows an increase in the size of the positive surface patch in the vicinity of residue 33. Likewise, the hydrophobicity is reduced due to the substitution to a highly polar residue as evidenced by the SAP score (Table 1). RMSF plot indicates good stability (FIG. 18), and MD analyses demonstrated good stability indicators (Table 2). Experimental DSF analysis on DM57 measured a high melting temperature (91° C.) (Table 7A), and a sensory panel indicated high sweetness and very high sweetness after heat treatment (Table 7A).

DM87 (D68N): The melting temperature of DM87 is 90, it is sweet and maintes it's sweetness after heat treatment (see Table 7A). an increased number of native contacts during simulations, as well as of intra-secondary structure backbone-mediated H-bonds of the helix and beta-sheet (Table 2). The helix of DM87 is on average more canonical than MNEI's helix (Table 2). This substation from a charged to neutral and polar residue decreases the hydrophobicity as exhibited by the SAP score (Table 1). Experimental DSF analysis on DM57 measured a high melting temperature (90° C.) (Table 7A), and a sensory panel indicated high sweetness and very high sweetness after heat treatment (Table 7A).

DM75 (E40): RMSF of this variant indicates good stability (FIG. 17). Based on MD simulations, DM75 displays good stability indicators (Table 2), e.g. its helix is on average more canonical than that of MNEI (Table 2). The hydrophobicity of this protein is reduced as indicated by the SAP score (Table 1). Experimental DSF analysis show high melting temperature (Table 7A).

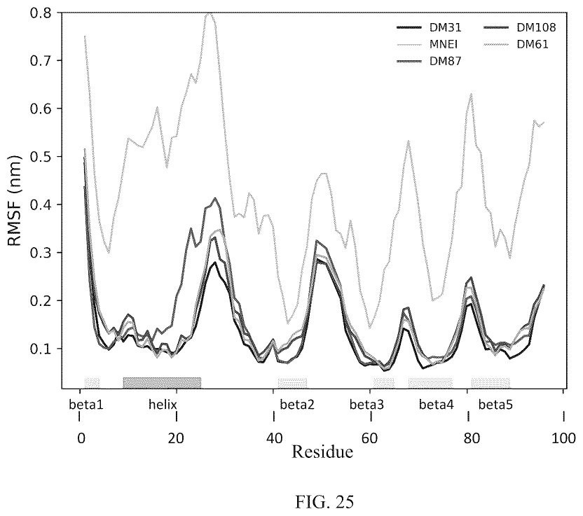

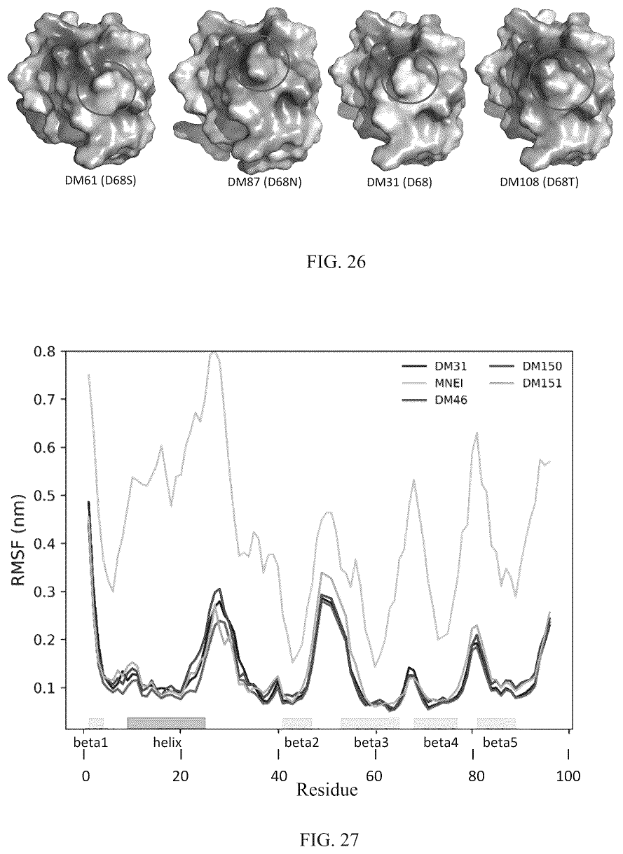

DM108 (D68T): the charge of the protein is more positive as result of the modification—FIG. 26. MD simulation at 433K show the protein keep its structure intact and still demonstrates higher stability than MNEI (FIG. 25). Surprisingly, the substitution from a charged residue to a neutral and polar residue causes a decrease in the hydrophobicity as evidenced by the SAP score (Table 1).

DM61 (D68S): MD simulations suggest that the RMSF of this DM is also better than DM87 and more similar to DM31 with the Lα2 loop being more stabilized (FIG. 25). The substitution from Asp to Ser at position 68 reduces the negative charge of the protein which is expected to improve long range interaction/attraction forces to the sweet receptor (FIG. 26).

D68N—see results in Example 1A.

DM65 (Q28K), DM66 (Q28R) and DM69 (Q28K+K25R) are 20% more sweet than DM31 (Table 7A). They also have high melting temperature with up to 97.5C for DM69, and their sweetness is maintained high after heat treatment, with up to 75% more than DM31 in DM66. DM65 and DM66 sweetness in 10 Bx is higher than DM31 (Table 7B and FIG. 38).

DM341—FIG. 41 presented APBS analysis of the DM341.

### Example 1C: Core Repacking

TABLE 5
Computational analyses for novel MNEI core variants and additional
variants, indicating voroMQA global score and ΔAUC.

| DM | ΔAUC | voroMQA |
|---|---|---|
| DM77 | 0.115 | 0.51 |
| DM491 | 0.957 | 0.5 |
| DM117 | −0.533 | 0.49 |
| DM84 | 0.753 | 0.48 |
| DM96 | 0.193 | 0.49 |
| DM506 | −0.657 | 0.48 |
| DM87 | 2.934 | 0.48 |
| DM508 | −0.485 | 0.49 |
| DM509 | 1.405 | 0.49 |
| DM46 | −1.139 | 0.4 |
| DM150 | 0.494 | 0.49 |
| DM151 | 0.751 | 0.48 |
| DM31 | 0 | 0.49 |
| DM498 | −0.677 | 0.5 |
| DM85 | 3.07 | 0.5 |
| DM91 | 1.06 | 0.49 |
| DM103 | 3.44 | 0.48 |
| DM432 | 2.28 | 0.48 |
| DM489 | 12.28 | 0.5 |
| DM505 | 14.07 | 0.5 |
| DM131 | 7.18 | 0.5 |
| DM132 | 2.76 | 0.5 |
| DM508 | −0.485 | 0.49 |
| DM509 | 1.405 | 0.49 |
| DM510 | 3.221 | 0.49 |
| DM452 | −0.61275 | 0.5 |
| DM70 | −0.91 | 0.5 |
| DM47 | 9.79 | 0.48 |
| DM420 | 13.21 | 0.49 |
| DM424 | −0.38 | 0.51 |

Core variants: Another region of the protein that was suggested by computational protein design as a design target is the core of the protein. Various proteins were designed with novel substitutions that lead to a core with better packing, and thus decreased accessibility to water, making the protein more stable. These positions are an outcome of the Rosetta design protocol done on core residues, along with rational design and visual analysis of the core, and additional packing and solvent accessibility analyses and MD simulations. In addition, position C41 was also targeted.

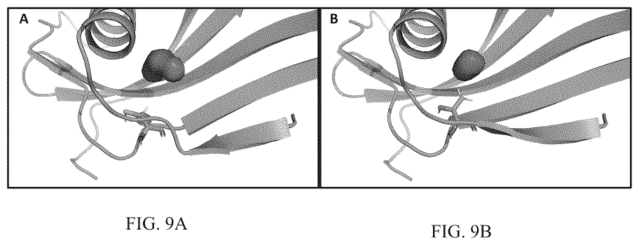

DM46 (C41T): C41T removes a cysteine residue, which possesses several undesired characteristics. The cysteine at position 41 is a risk for disulfide bond formation and nucleophilic attack causing the release of the sulfur atom, thus potentially reducing the protein's shelf life. In addition, cysteine is the second rarest amino acid (after tryptophan) and thus results in lower expression levels due to decreased tRNA available for the codon of this amino acid. The substitution of cysteine solves these problems. This replacement also leads to a reduction of a proximal cavity found in the hydrophobic core of the protein (FIG. 9, A: C41 in sticks B: T41 in sticks, inner cavity is shown in pink). A better packed core is expected to be more stable due to the increased rigidity and the decreased access to water molecules. DM46 also displays increased heat resistance (Table 7A) and is predicted to be stable during MD simulations at 433K (FIG. 27).

DM150 (C41A): displays increased heat resistance and good melting temperature according to DSF analysis (Table 7A), and is predicted to be stable during MD simulations at 433K (FIG. 27). Surprisingly, this protein exhibits dramatically reduced hydrophobicity (see SAP score in Table 1) despite the substitution from a polar residue to a hydrophobic residue.

DM151 (C41S): displays increased heat resistance (Table 7A) and is predicted to be as stable as DM31 during MD simulations at 433K (FIG. 27). This DM along with DM150 exhibits dramatically reduced hydrophobicity (SAP score, Table 1).

DM85 (A73V): Experimental DSF analysis on DM85 measured a high melting temperature 94.5° C. (Table 7A). Surprisingly, the substitution from a small hydrophobic residue to a larger one led to decreased hydrophobicity as measured by the SAP score (Table 1).

DM77 (A19V): displays increased heat resistance (Table 7A) and is predicted to have improved packing in comparison to DM31 (higher voroMQA global score, Table 5). Surprisingly, the substitution from a small hydrophobic residue to a larger one led to decreased hydrophobicity as measured by the SAP score (Table 1).

DM96 (V20I): displays increased sweetness at 6 brix and increased heat resistance, along with good melting temperature (Table 7A). RMSF plot shows good stability (FIG. 31). FIG. 32 shows APBS analysis of DM96.

DM117 (V20I+V64I): The variant displays a lower RMSF in comparison to that of DM84, DM96 and DM31 (FIG. 328. Is more packed than DM84, with higher voroMQA score (Table 5). DM117 has lower REU than DM31 (Table 1). Surprisingly, hydrophobicity (SAP score, Table 1) was also lower for these substitutions despite the replacement to larger hydrophobic residues at both positions.

DM498 (C41S+V64I+A73F+I75L+F89V): is a result of core design as described above. The variant displays a lower RMSF in comparison to DM31 (FIG. 29). DM498 displays improved packing compared to DM31 (Table 5). The SAP score for this DM is dramatically low as compared to the DMs analyzed in Table 1.

DM491 (V20I+C41A+A73F+I75L+F89M): is a result of core design as described above. DM491 displays improved packing in comparison to DM31 (Table 5). It has lower RMSF in comparison to DM31 (FIG. 28, Table 5). DM492 has lower REU than DM31 (Table 1). These substitutions lowered the overall hydrophobicity of the protein as evidenced by the SAP score (Table 1).

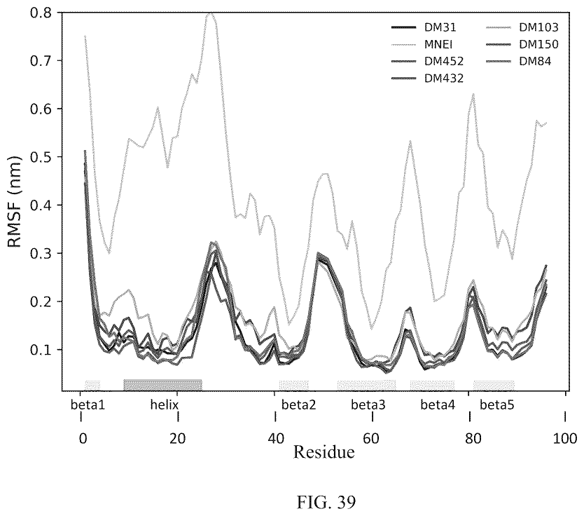

DM452 (C41A+V64L+I75V): is an improvement upon DM432, removing G16A known to harm stability. The variant displays good stability in RMSF plot (FIG. 39). In addition, this DM exhibits lower hydrophobicity as evidenced by its SAP score (Table 1).

DM489 (V20I+C41V+A73V): displays improved packing in comparison to DM31 (Table 5), and a low REU value, indicating stability (Table 1).

DM509 (K25R+V20I): described in the helix-capping section. It is a combination of K25R and V20I. It surprisingly exhibits lower hydrophobicity despite chemically similar substitutions (Table 1).

DM508 (V20I+Q28K): described in the helix-capping section. It is a combination of V20I and Q28K. The variant displays good stability in RMSF plot (FIG. 31). Q28K is expected to add sweetness, due to an increase in the positive surface local electrostatics potential, as indicated by APBS analysis (see FIG. 32). In addition, the protein presents a low SAP score (Table 1), and increased packing for position 28 according to VoroMQA analysis (FIG. 23).

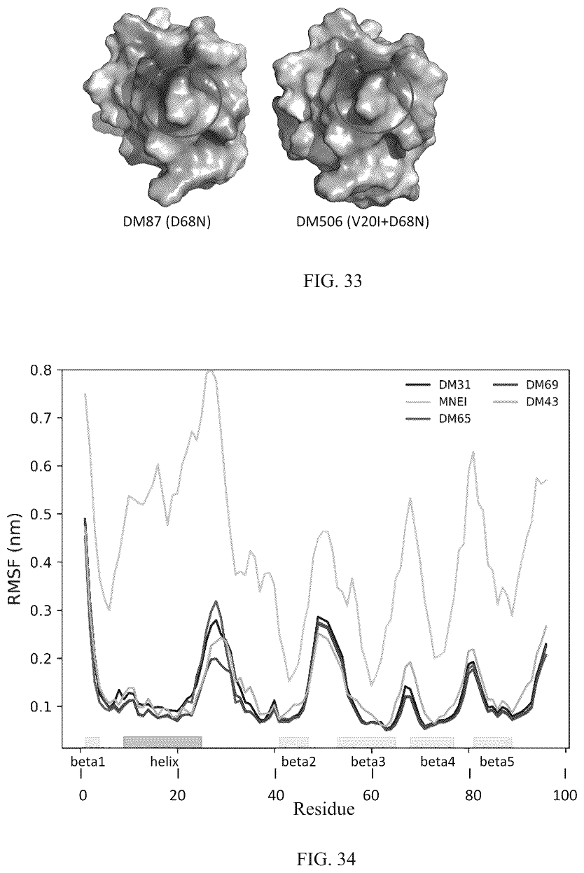

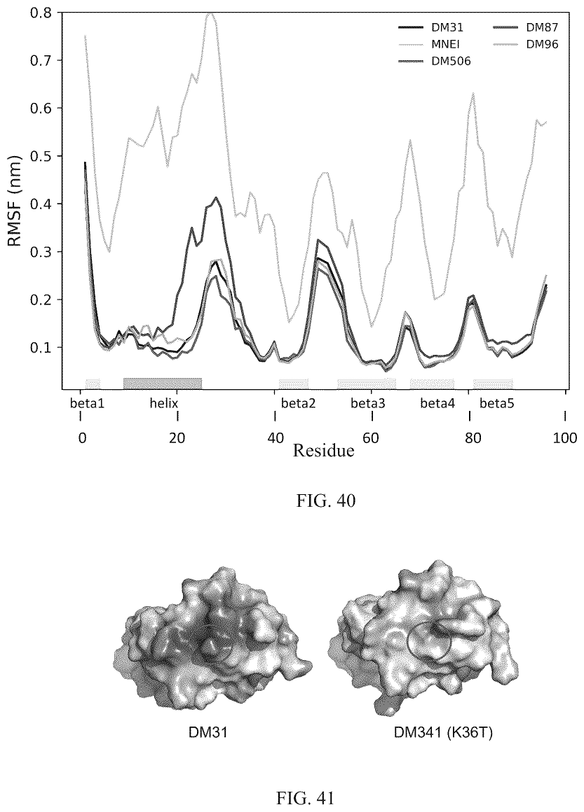

DM506 (V20I+D68N): The electrostatic potential shown by APBS (FIG. 33) indicates that DM506 is expected to be as sweet as DM87. MD plots and analyses indicate good stability (FIG. 40, Tables 2, 4, 5). In addition, the protein shows a reduction in SAP score (Table 1). FIG. 40 presents Average RMSF (Root Mean Square Fluctuations) of DM31, MNEI, DM506 (V20I+D68N), DM87 (D68N), and DM96 (V20I), indicating the stability of DM506.

DM505 (A19V+Q28K): is a combination of DM77 (A19V) and DM65 (Q28K). It is expected to have better packing than DM31 (Table 5).

DM47 (R3IT): Rosetta Energy Unit (REU) scoring energy of this substitution results in a lower energy (−322.6 REU) compared to DM31, as well as a lower SAP score (Table 1). The substitution makes the hydrophobic “neck” of residue 31 (which stretches between C-beta to C-delta atoms) shorter. Since the position is solvent accessible, it was suggested that R3IT also reduces the overall hydrophobicity and the risk for aggregation (Samish I., 2017, Methods Mol Biol., 1529, 3-19). Tm of DM47 was determined experimentally via DSF, resulting in improved stability comparing to DM31 (Table 7A).

FIG. 13 shows RMSF plot of proteins DM84, DM96, DM77, DM85, exemplifying their stability. Table 2 shows additional MD based measures for these core variants, indicating good stability.

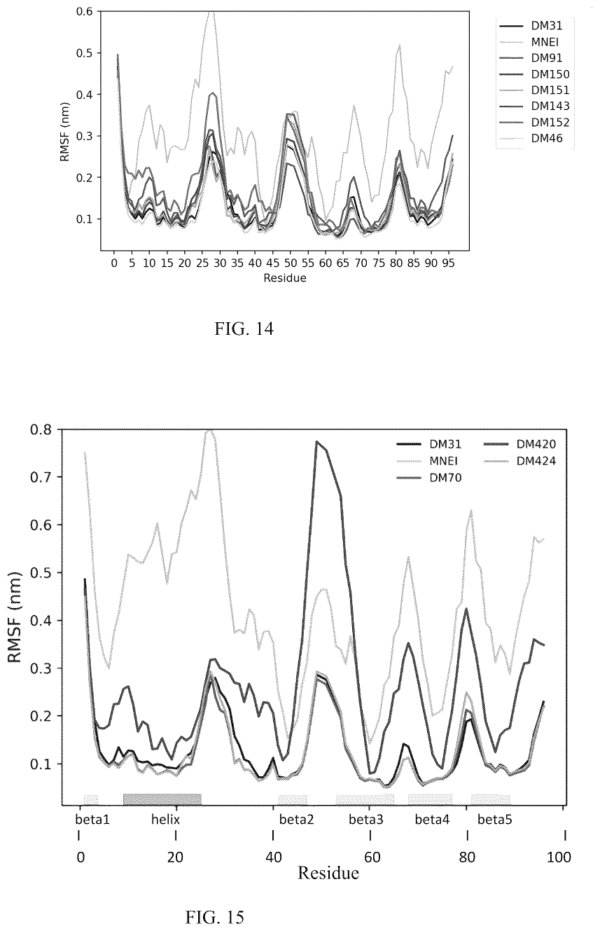

FIG. 14 presents RMSF plot for proteins DM91, DM150, DM151, DM143, DM152 and DM46, indicating their stability. Table 2 shows additional MD based measures for these core variants, indicating good stability.

### Example 1D Additional Variants

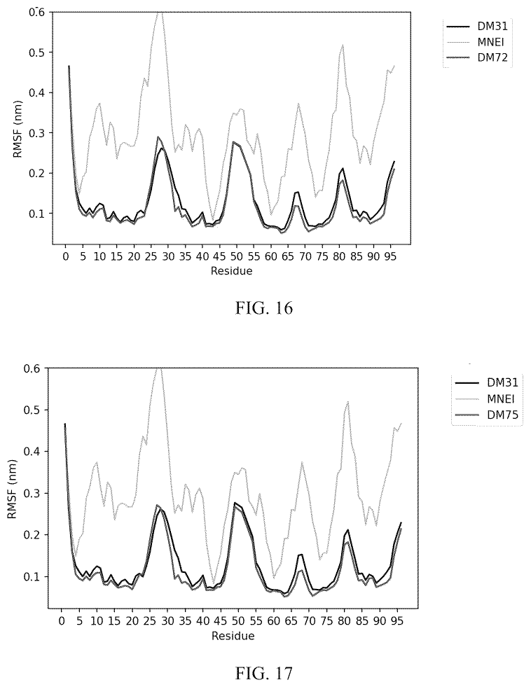

DM72: N35T, while maintaining the polarity, is replacing asparagine with a smaller amino acid, thus avoiding the effect of exposed hydrophobic atoms. As can be seen in Table 1, Rosetta predicts N35T to be favorable. Molecular dynamics simulations of MNEI and DM72 showed that DM72 is generally more stable and rigid than MNEI. At 433K (FIG. 16, Table 2 & Table 4) all the regions of DM72 are more stable than MNEI. Experimental DSF analysis on DM72 measured high melting temperature (99° C.) (Table 7A).

DM70 (R84L): Arginine, being a long positively charged amino acid, has a hydrophobic section in its side-chain (C-beta to C-delta atoms). Based on crystal data, it was suggested that in MNEI and DM31, R84L is mostly exposed, thereby resulting in an exposed hydrophobic stretch of atoms. It was also suggested that the substitution R84L is expected to allow better protein packing rather than extending a hydrophobic “neck” (which stretches between the C-beta and C-delta atoms). Rosetta analysis suggests that indeed R84L results in a significantly more stable protein (Table 1). FIG. 15 shows the RMSF of DM70 in molecular dynamics simulations at 433K, demonstrating stability. In addition, the number of intra-secondary structure backbone-mediated H-bonds of the helix and beta-sheet of DM70 is elevated (Table 2). RMSD values of the loops (Loop A2(residues 26-35) and L23) and the helix in DM70 indicate stability. DM70 helix is on average more canonical than MNEI's helix. (Table 2). Additionally, the melting temperature (Tm) of DM70 was determined experimentally via DSF, indicating good thermostability (94° C., see Table 7A).

DM420: R84Y is mutation that is expected to increase packing of the protein together with keeping hydrogen bonds that were with the original residue. Rosetta's REU score indicates good stability (Table 1). Surprisingly, this substitution led to decreased hydrophobicity despite a substitution to a more hyrophobic residue (Y), as indicated by SAP score (Table 1).

DM424: R84I is mutation that is designed for better packing of the protein. FIG. 15 shows the RMSF of DM424 and DM31 in molecular dynamics simulations at 433K, showing that DM424's loops are as stable and rigid. The protein is predicted to be more packed than DM31 (Table 5).

FIG. 15 present average RMSF (Root Mean Square Fluctuations) of DM31, MNEI, DM70 (R84L), DM420 (R84Y) and DM424 (R84I), all include a substitution in residue 84. The RMSF indicates good stability.

### Example 2: Cloning, Expression, and Characterization of MNEI Designer Proteins

Recombinant MNEI proteins were produced in E. coli BL21(DE3+) under a T7 promoter induced with Isopropyl β-D-1-thiogalactopyranoside (IPTG). Using this system, MNEI proteins were expressed as cytosolic protein (soluble fraction) in a high-density fermentation process. Designer MNEI (DM) are designed proteins with up to 11% amino acid substitutions.

All DMs were produced in E. Coli fermentation and purified to a level of >95%.

### Cloning

Site-directed mutagenesis (SDM), was used for creating the DMs.

TABLE 6A
Sequences of the modified proteins and MNEI

| SEQ ID | |
|---|---|
| NO: | Sequence |
| SEQ ID | GNWEIIDIGPFTQNLGKFAVDEANKIGQYGRLTFNK |
| NO: 1 | VIRPTMKKTIYENGEIKGYEYQLYVRASDKIFRADIS |
| (DM46_ | EDYKTRGRKLLRFNGPVPPP |
| C41T) | |
| SEQ ID | GNWEIIDIGPFTQNLGKFAVDEANKIGQYGTLTFNK |
| NO: 2 | VIRPCMKKTIYENGEIKGYEYQLYVRASDKIFRADIS |
| (DM47_ | EDYKTRGRKLLRFNGPVPPP |
| R31T) | |
| SEQ ID | GNWEIIDIGPFTQNLGKFAVDEANKIGQYGRLTFNK |
| NO: 3 | VIRPCMKKTIYENGEIKGYEYQLYVRASDKIFRADIS |
| (DM70_ | EDYKTRGLKLLRFNGPVPPP |
| R84L) | |
| SEQ ID | GNWEIIDIGPFTQNLGKFAVDEANKTGQYGRLTFNK |
| NO: 4 | VIRPCMKKTIYENGEIKGYEYQLYVRASDKIFRADIS |
| (DM42_ | EDYKTRGRKLLRFNGPVPPP |
| I26T) | |
| SEQ ID | GNWEIIDIGPFTQNLGKFAVDEANRIGQYGRLTFNK |
| NO: 5 | VIRPCMKKTIYENGEIKGYEYQLYVRASDKIFRADIS |
| (DM43_ | EDYKTRGRKLLRFNGPVPPP |
| K25R) | |
| SEQ ID | GNWEIIDIGPFTQNLGKFAVDEANKIGQYGRLTFTK |
| NO: 6 | VIRPCMKKTIYENGEIKGYEYQLYVRASDKIFRADIS |
| (DM72_ | EDYKTRGRKLLRFNGPVPPPd |
| N35T) | |
| SEQ ID | GNWEIIDIGPFTQNLGKFAVDEANKIGQYGRLTFNK |
| NO: 7 | VIRPCMKKTIYENGEIKGYEYQLYVRASDKIFRADIS |
| (DM31) | EDYKTRGRKLLRFNGPVPPP |
| SEQ ID | GEWEIIDIGPFTQNLGKFAVDEENKIGQYGRLTFNKV |
| NO: 8 | IRPCMKKTIYENEGFREIKGYEYQLYVYASDKLFRA |
| (MNEI) | DISEDYKTRGRKLLRFNGPVPPP |
| SEQ ID | GNWEIIDIGPFTQNLGKFAVDEANKIGQYGRLTFNK |
| NO: 9 | VIRPCMKKTIYENEGFREIKGYEYQLYVRASDKLFR |
| (DM09) | ADISEDYKTRGRKLLRFNGPVPPP |

TABLE 6B
Sequences of the modified proteins and MNEI

| SEQ ID | |
|---|---|
| NO: | Sequence |
| SEQ ID | GNWEIIDIGPFTQNLGKFAVDEANKIGQYGRLTFNK |
| NO: 10 | VIRPCMKKTIYENEGFREIKGYEYQLYVRASDKIFRA |
| (DM14) | DISEDYKTRGRKLLRFNGPVPPP |
| SEQ ID | GEWEIIDIGPFTQNLGKFAVDEENKIGQYGRLTFNKV |
| NO: 11 | IRPCMKKTIYENGEIKGYEYQLYVYASDKLFRADISE |
| (DM29) | DYKTRGRKLLRFNGPVPPP |
| SEQ ID | GNWEIIDIGPFTQNLGKFAVDEANKSGQYGRLTFNK |
| NO: 12 | VIRPCMKKTIYENEGFREIKGYEYQLYVRASDKIFRA |
| (DM41) | DISEDYKTRGRKLLRFNGPVPPP |
| SEQ ID | GEWEIIDIGPFTQNLGKFAVDEENKTGQYGRLTFNK |
| NO: 13 | VIRPCMKKTIYENEGFREIKGYEYQLYVYASDKLFR |
| (DM42b) | ADISEDYKTRGRKLLRFNGPVPPP |
| SEQ ID | GNWEIIDIGPFTQNLGKFAVDEANKIGKYGRLTFNK |
| NO: 14 | VIRPCMKKTIYENGEIKGYEYQLYVRASDKIFRADIS |
| (DM65) | EDYKTRGRKLLRFNGPVPPP |
| SEQ ID | GNWEIIDIGPFTQNLGKFAVDEANKIGRYGRLTFNK |
| NO: 15 | VIRPCMKKTIYENGEIKGYEYQLYVRASDKIFRADIS |
| (DM66) | EDYKTRGRKLLRFNGPVPPP |
| SEQ ID | GNWEIIDIGPFTQNLGKFAVDEANKIGRYGTLTFNK |
| NO: 18 | VIRPCMKKTIYENGEIKGYEYQLYVRASDKIFRADIS |
| (DM73) | EDYKTRGRKLLRFNGPVPPP |
| SEQ ID | GNWEIIDIGPFTQNLGKFAVDEANKTGKYGRLTFNK |
| NO: 19 | VIRPCMKKTIYENGEIKGYEYQLYVRASDKIFRADIS |
| (DM92) | EDYKTRGRKLLRFNGPVPPP |
| SEQ ID | GNWEIIDIGPFTQNLGKFAVDEANKIGQYGRLTFNK |
| NO: 20 | VIRPCMKKTIYENGEIKGYEYQLYVRASDKIFRADLS |
| (DM103) | EDYKTRGRKLLRFNGPVPPP |
| SEQ ID | GNWEIIDIGPFTQNLGKFAVDEANKWGEYGRLTFNK |
| NO: 21 | VIRPCMKKTIYENGEIKGYEYQLYVRASDKIFRADIS |
| (DM115) | EDYKTRGRKLLRFNGPVPPP |
| SEQ ID | GNWEIIDIGPFTQNLGKFAVDEANKWGKYGRLTFN |
| NO: 22 | KVIRPCMKKTIYENGEIKGYEYQLYVRASDKIFRADI |
| (DM116) | SEDYKTRGRKLLRFNGPVPPP |
| SEQ ID | GNWEIIDIGPFTQNLGKFAVDEANKWGQYGRLTFN |
| NO: 23 | KVIRPCMKKTIYENGEIKGYEYQLYVRASDKIFRADI |
| (DM144) | SEDYKTRGRKLLRFNGPVPPP |
| SEQ ID | GNWEIIDIGPFTQNLGKFAVDEANKIGEYGRLTFNK |
| NO: 24 | VIRPCMKKTIYENGEIKGYEYQLYVRASDKIFRADIS |
| (DM145) | EDYKTRGRKLLRFNGPVPPP |
| SEQ ID | GNWEIIDIGPFTQNLGKFVVDEANKIGQYGRLTFNK |
| NO: 25 | VIRPCMKKTIYENGEIKGYEYQLYVRASDKIFRADIS |
| (DM77) | EDYKTRGRKLLRFNGPVPPP |
| SEQ ID | GNWEIIDIGPFTQNLGKFAVDEANKIGQYGRLTFNK |
| NO: 26 | VIRPCMKKTIYENGEIKGYEYQLYIRASDKIFRADISE |
| (DM84) | DYKTRGRKLLRFNGPVPPP |
| SEQ ID | GNWEIIDIGPFTQNLGKFAVDEANKIGQYGRLTFNK |
| NO: 27 | VIRPCMKKTIYENGEIKGYEYQLYVRASDKIFRVDIS |
| (DM85) | EDYKTRGRKLLRFNGPVPPP |
| SEQ ID | GNWEIIDIGPFTQNLGKFAVDEANKIGQYGRLTFNK |
| NO: 28 | VIRPVMKKTIYENGEIKGYEYQLYVRASDKIFRADIS |
| (DM91) | EDYKTRGRKLLRFNGPVPPP |
| SEQ ID | GNWEIIDIGPFTQNLGKFAVDEANKIGQYGRLTFNK |
| NO: 29 | VIRPCMKKTIYENGEIKGYEYQLYVRASDKIFRADIS |
| (DM104) | EDYKTRGRKLLRMNGPVPPP |
| SEQ ID | GNWEIIDIGPFTQNLGKFAVDEANKIGQYGRLTFNK |
| NO: 30 | VIRPCMKKTIYENGEIKGYEYQLYVRASDKIFRFDIS |
| (DM131) | EDYKTRGRKLLRFNGPVPPP |
| SEQ ID | GNWEIIDIGPFTQNLGKFAVDEANKIGQYGRLTFNK |
| NO: 31 | VIRPCMKKTIYENGEIKGYEYQLYVRASDKIFRFDIS |
| (DM132) | EDYKTRGRKLLRMNGPVPPP |
| SEQ ID | GNWEIIDIGPFTQNLGKFAVDEANKIGQYGRLTFNK |
| NO: 32 | VIRPAMKKTIYENGEIKGYEYQLYVRASDKIFRADIS |
| (DM150) | EDYKTRGRKLLRFNGPVPPP |
| SEQ ID | GNWEIIDIGPFTQNLGKFAVDEANKIGQYGRLTFNK |
| NO: 33 | VIRPSMKKTIYENGEIKGYEYQLYVRASDKIFRADIS |
| (DM151) | EDYKTRGRKLLRFNGPVPPP |
| SEQ ID | GNWEIIDIGPFTQNLGKFAVDEANKIGQYGRLRFNK |
| NO: 34 | VIRPCMKKTIYENEGFREIKGYEYQLYVRASDKIFRA |
| (DM57) | DISEDYKTRGRKLLRFNGPVPPP |
| SEQ ID | GNWQIIDIGPFTQNLGKFAVDEANKIGQYGRLTFNK |
| NO: 35 | VIRPCMKKTIYENGEIKGYEYQLYVRASDKIFRADIS |
| (DM75) | EDYKTRGRKLLRFNGPVPPP |
| SEQ ID | GNWEIIDIGPFTQNLGKFAVDEANKIGQYGRLTFNK |
| NO: 36 | VIRPCMKKTIYENGEIKGYEYQLYVRASNKIFRADIS |
| (DM87) | EDYKTRGRKLLRFNGPVPPP |
| SEQ ID | GNWEIIDIGPFTQNLGKFAVDEANKIGQYGRLTFNK |
| NO: 37 | VIRPCMKKTIYENGEIKGYEYQLYVRASNKIFRADIS |
| (DM93) | EDYKTRGLKLLRFNGPVPPP |
| SEQ ID | GNWEIIDIGPFTQNLGKFAIDEANKIGQYGRLTFNKV |
| NO: 38 | IRPCMKKTIYENGEIKGYEYQLYVRASDKIFRADISE |
| (DM96) | DYKTRGRKLLRFNGPVPPP |
| SEQ ID | GEWEIIDIGPFTQNLGKFAVDEENKIGQYGRLTFNKV |
| NO: 39 | IRPCMKKTIYENEGFREIKGYEYQLYVYASNKLFRA |
| (DM157) | DISEDYKTRGRKLLRFNGPVPPP |
| SEQ ID | GNWEIIDIGPFTQNLGKFAVDEANKIGQYGRLTFNK |
| NO: 40 | VIRPCMKKTIYENGEIKGYEYQLYVRASDKLFRADIS |
| (DM28) | EDYKTRGRKLLRFNGPVPPP |
| SEQ ID | GNWEIIDIGPFTQNLGKFAVDEANKIGQYGTLTFNK |
| NO: 41 | VIRPCMKKTIYENGEIKGYEYQLYVRASNKIFRADIS |
| DM94 | EDYKTRGRKLLRFNGPVPPP |
| SEQ ID | GNWEIIDIGPFTQNLGKFAVDEANKIGQYGTLTFNK |
| NO: 42 | VIRPCMKKTIYENGEIKGYEYQLYVRASDKIFRADIS |
| DM97 | EDYKTRGLKLLRFNGPVPPP |
| SEQ ID | GEWEIIDIGPFTQNLGKFAVDEENKIGQYGRLTFNKV |
| NO: 43 | IRPCMKKTIYENGEIKGYEYNLYVYASDKIFRADISE |
| DM99 | DYKTRGRKLLRFNGPVPPP |
| SEQ ID | GNWEIIDIGPFVQNLGKFAVDEANKIGQYGRLTFNK |
| NO: 44 | VIRPVMKKTIYENGEIKGYEYQLYVRASDKIFRADIS |
| DM143 | EDYKTRGRKLLRFNGPVPPP |
| SEQ ID | GNWEIIDIGPFVQNLGKFAVDEANKIGQYGRLTFNK |
| NO: 45 | VIRPCMKKTIYENGEIKGYEYQLYVRASDKIFRADIS |
| DM152 | EDYKTRGRKLLRFNGPVPPP |
| SEQ ID | GNWEIIDIGPFTQNLGKFAVDEANRIGKYGRLTFNK |
| NO: 17 | VIRPCMKKTIYENGEIKGYEYQLYVRASDKIFRADIS |
| DM69 | EDYKTRGRKLLRFNGPVPPP |

TABLE 6C
Sequences of the modified proteins

| SEQ ID | |
|---|---|
| NO: | Sequence |
| SEQ ID | GNWEIIDIGPFTQNLGKFAVDEANKIGQYGRLTFNK |
| NO: | VIRPCMKKTIY |
| DM108 | ENGEIKGYEYQLYVRASTKIFRADISEDYKTRGRKLL |
| | RFNGPVPPP |
| SEQ ID | GNWEIIDIGPFTQNLGKFAIDEANKIGQYGRLTFNKV |
| NO: | IRPCMKKTIYENGEIKGYEYQLYIRASDKIFRADISED |
| DM117 | YKTRGRKLLRFNGPVPPP |
| SEQ ID | GNWEIIDIGPFTQNLGKFAVDEANKIGSYGRLTFNKV |
| NO: | IRPCMKKTIYENGEIKGYEYQLYVRASDKIFRADISE |
| DM330 | DYKTRGRKLLRFNGPVPPP |
| SEQ ID | GNWEIIDIGPFTQNLGKFAVDEANRIGQYGRLTFNK |
| NO: | VIRPCMKKTIYENGEIKGYEYQLYVRASNKIFRADIS |
| DM464 | EDYKTRGRKLLRFNGPVPPP |
| SEQ ID | GNWEIIDIGPFTQNLGKFAIDEANKIGKYGRLTFNKV |
| NO: | IRPCMKKTIYENGEIKGYEYQLYVRASDKIFRADISE |
| DM508 | DYKTRGRKLLRFNGPVPPP |
| SEQ ID | GNWEIIDIGPFTQNLGKFAIDEANRIGQYGRLTFNKV |
| NO: | IRPCMKKTIYENGEIKGYEYQLYVRASDKIFRADISE |
| DM509 | DYKTRGRKLLRFNGPVPPP |
| SEQ ID | GNWEIIDIGPFTQNLGKFAIDEANKIGQYGRLTFNKV |
| NO: | IRPCMKKTIYENGEIKGYEYQLYVRASNKIFRADISE |
| DM506 | DYKTRGRKLLRFNGPVPPP |
| SEQ ID | GNWEIIDIGPFTQNLGKFAIDEANKIGQYGRLTFNKV |
| NO: | IRPAMKKTIYENGEIKGYEYQLYVRASDKIFRFDLSE |
| DM491 | DYKTRGRKLLRMNGPVPPP |
| SEQ ID | GNWEIIDIGPFTQNLGKFAVDEANKIGQYGRLTFNK |
| NO: | VIRPSMKKTIYENGEIKGYEYQLYIRASDKIFRFDLSE |
| DM498 | DYKTRGRKLLRVNGPVPPP |
| DM341 | GNWEIIDIGPFTQNLGKFAVDEANKIGQYGRLTFNT |
| | VIRPCMKKTIYENGEIKGYEYQLYVRASDKIFRADIS |
| | EDYKTRGRKLLRFNGPVPPP |
| DM420 | GNWEIIDIGPFTQNLGKFAVDEANKIGQYGRLTFNK |
| | VIRPCMKKTIYENGEIKGYEYQLYVRASDKIFRADIS |
| | EDYKTRGYKLLRFNGPVPPP |
| DM424 | GNWEIIDIGPFTQNLGKFAVDEANKIGQYGRLTFNK |
| | VIRPCMKKTIYENGEIKGYEYQLYVRASDKIFRADIS |
| | EDYKTRGIKLLRFNGPVPPP |
| DM432 | GNWEIIDIGPFTQNLAKFAVDEANKIGQYGRLTFNK |
| | VIRPAMKKTIYENGEIKGYEYQLYLRASDKIFRADVS |
| | EDYKTRGRKLLRFNGPVPPP |
| DM452 | GNWEIIDIGPFTQNLGKFAVNEANKIGQYGRLTFNK |
| | VIRPCMKKTIYENGEIKGYEYQLYVRASDKIFRADIS |
| | EDYKTRGRKLLRFNGPVPPP |
| DM489 | GNWEIIDIGPFTQNLGKFAIDEANKIGQYGRLTFNKV |
| | IRPVMKKTIYENGEIKGYEYQLYVRASDKIFRVDISE |
| | DYKTRGRKLLRFNGPVPPP |
| DM505 | GNWEIIDIGPFTQNLGKFVVDEANKIGKYGRLTFNK |
| | VIRPCMKKTIYENGEIKGYEYQLYVRASDKIFRADIS |
| | EDYKTRGRKLLRFNGPVPPP |
| DM510 | GNWEIIDIGPFTQNLGKFVVDEANRIGQYGRLTFNK |
| | VIRPCMKKTIYENGEIKGYEYQLYVRASDKIFRADIS |
| | EDYKTRGRKLLRFNGPVPPP |

### Fermentation

All DM clones were subjected to fermentation in 3 L vessels using the Sartorius BioStatB system or 2 L vessels of Solaris Jupiter system. Some DM clones were produced by outsourcing at VTT (Finland) and SciVac (Israel) All fermentations followed a protocol based on “High cell-density fermentation of Escherichia coli” by Arie Geerlof—EMBL Hamburg 29 Jan. 2008.

### Purification

All DM samples were purified using the following steps:
1. Lysis by pressure homogenizer.
2. Capture of the protein on a multimode resin, elution with increasing NaCl concentration in the same buffer.
3. At least one polishing step using resins from the following groups:
1. Ion exchange.
2. Hydrophobic interaction.
3. Size exclusion.
4. Final microbial filtration (0.2 um) and storage at −20° C.

### Characterization

### Purification Level

Purification level was evaluated using gel electrophoresis followed by Coomassie staining and densitometry analysis and by SEC-HPLC. Analysis by densitometry was conducted by running 20 g/lane of each protein, using a a pure DM standard curve of 50-500 ng/lane. In all samples, maximal contamination reached 3% (i.e., 97% purity).

### Example 3: Characterization of the Modified Proteins

### This Example Included Sensory Evaluation of Sweet Taste Intensity and Stability

### Shelf-Life Stability of DM31 was Tested (5 Bx Equivalent in Buffer Citrate).

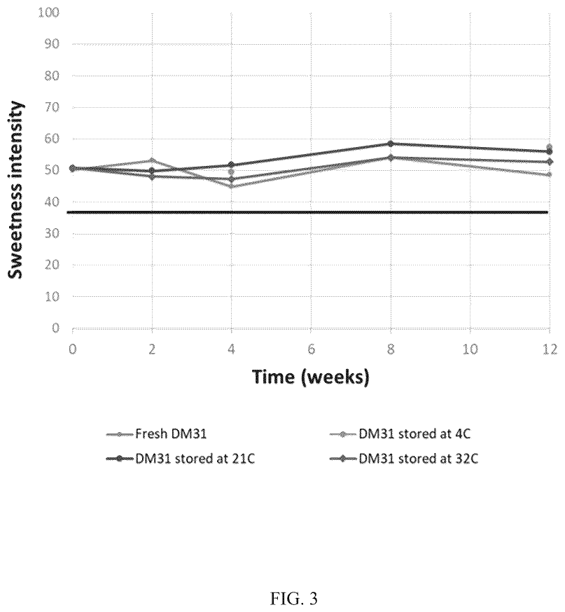

As shown in FIG. 3, the sweetness intensity of DM31 is stable after 12 weeks both at 21° C. and 32° C.

### Heat Stability of DM31 in Rice Flour Base (40% Moisture)

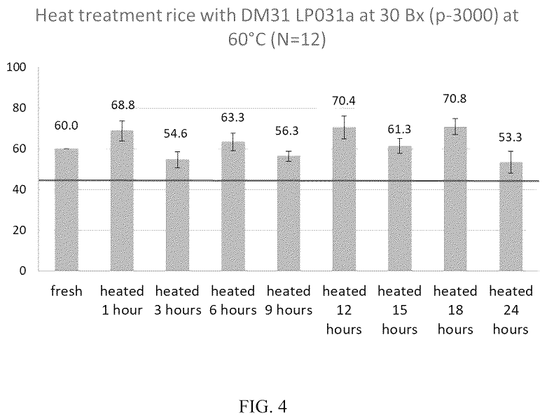

Heat stability of DM31 was tested in rice flour with water at 40% moisture content. DM31 was added to the water at a concentration of 30 Bx° equivalent. The rice flour with the sweet protein was dried at 60° C. for 1, 3, 6, 9, 12, 15, 18 and 24 hours in closed jars. Sweetness intensity on a 0-100 scale, while 100 define as 8° Bx equivalent, was compared to a fresh product by Amai's expert panel. The fresh product was defined in the panel as 60 on the scale. As shown in FIG. 4, the sweetness intensity of DM31 is stable at 60° C. for 24 hours.

Sweetness Intensity of DM31 after Heat Treatment of Rice Flour with 40% Moisture Content

Heat stability of DM31 was tested in rice flour with water at 40% moisture content. DM31 was added to the water at a concentration of 30 Bx° equivalent. The rice flour with the sweet protein was dried at 60° C. and 70° C. for 18 hours in open jars.

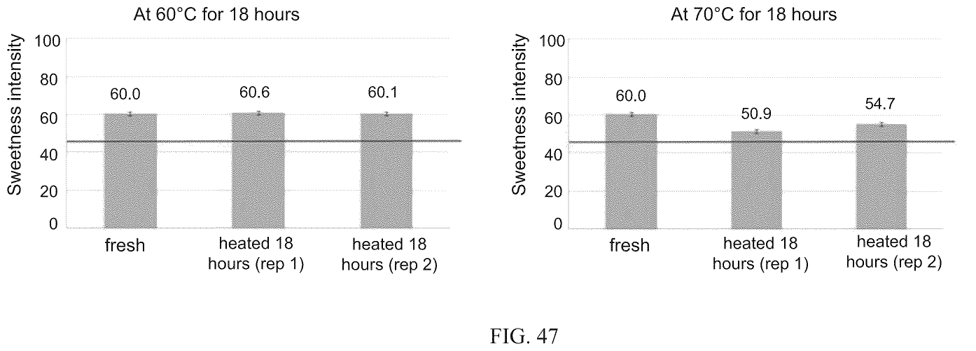

As shown in FIG. 47, the sweetness intensity of DM31 in rice flour with 40% moisture content is stable after heat treatment at 60° C. and 70° C. for 18 hours in open plates and reconstitution with water.

### Heat Stability of DM31 in Powder Form

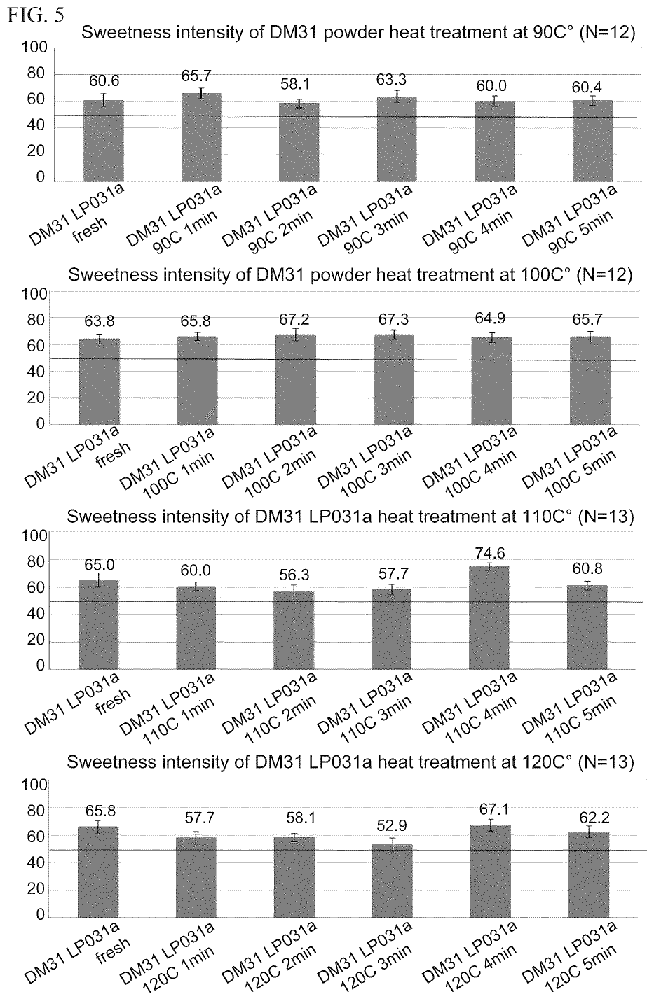

Heat stability of the powdered DM31 was tested at high temperatures for a time of 1-5 minutes. The sweet protein powder was dried at 90° C., 100° C., 110° C. and 120° C. for 1,2,3,4,5 minutes. The samples were suspended with water and were tested by Amai's expert sensory panel. Amai's expert panel evaluated the sweetness intensity on 0-100 scale compared to a fresh product. As shown in FIG. 5, the sweetness intensity of DM31 in a powder form is stable up to 120° C. for 5 minutes.

### Sweetness Evaluation

A professional sensory panel includes expert supertaster panelists (as described below) who were calibrated first with sugar solutions on a scale of 0-100 (magnitude estimation), with 0=not sweet at all and 100=very sweet. After calibration, tasters graded the tested samples in a blind manner on the same scale according to the validated tasting protocol. Linear scale for sucrose was obtained in concentrations of 2° Bx, 4° Bx, 6° Bx, and 8° Bx.

$$Brix ( Bx ) = gr / 100 ml .$$

Initial sweetness evaluation was conducted by comparing to sugar at 6° Bx, and the new selected DMs in each test, at a selected potency X4000. All dilutions were done in water only. Samples were evaluated by an expert panel on different occasions, using 6° Bx sucrose as controls.

### Dsf and DSC Analysis—Heat Sensitivity

Tm-value was determined by Differential Scanning Fluorimetry (DSF) using Nanotemper Prometheus Panta. DSF is a method for easy, rapid, and accurate analysis of protein stability and aggregation. DSF detects changes in the fluorescence of tryptophan and tyrosine residues in the protein. The fluorescence of tryptophan and tyrosine residues is strongly dependent on their close surroundings. A change in protein conformation will be reflected as a fluorescence change. The 1 st derivate of the fluorescence ratio (330 nm/350 nm) is used to determine the inflection point. Since no secondary reporter fluorophores are required, protein solutions can be analyzed independently of buffer compositions and over a concentration range of 250 mg/mi down to 10 μg/ml. DMs were analyzed at a concentration of 0.5 mg/ml in a 5 mM Citrate buffer pH 6.

### Results:

TABLE 7A
Experimental results for polypeptide variants

| | | | Heat treatment | |
|---|---|---|---|---|
| | | Sweetness | 95Â° C. 30 min | |
| DM | Modification | vs DM31 | vs DM31 | Tm |
| DM65 | Q28K | 1.2 | 1.26 | 93.7 |
| DM57 | T33R | 1.09 | 1.15 | 91 |
| DM43 | K25R | 1.06 | 1.27 | 94 |
| DM96 | V20I | 1.06 | 1.18 | 92.5 |
| DM87 | D68N | 1.05 | 1.23 | 90 |
| DM77 | A19V | 1.03 | 1.13 | 88.1 |
| DM150 | C41A | 0.98 | 1.17 | |
| DM151 | C41S | 0.96 | 1.29 | 88 |
| DM46 | C41T | 0.95 | 1.08 | 88.8 |
| DM66 | Q28R | 1.2 | 1.75 | 95 |
| DM69 | K25R + Q28K | 1.2 | | 97.5 |
| DM41 | I26S | 0.89 | | 94 |
| DM72 | N35T | 0.97 | | 99 |
| DM42 | I26T | 1 | 0.58 | 99 |
| DM70 | R84L | 0.57 | | 94 |
| DM85 | A73V | 0.64 | | 94.5 |
| DM75 | E4Q | 0.75 | | 93.6 |
| DM47 | R31T | 0.69 | | 97 |

TABLE 7B
Experimental results for polypeptide variants at 10 brix

| | | Sweetness vs | P-value |
|---|---|---|---|
| DM | Substitutions | DM31 10 Brix | 10 Brix |
| 31 | E2N + E23A + | 1 | |
| | Y65R + L70I | | |
| 65 | Q28K | 1.18 | 0.001285 |
| 66 | Q28R | 1.14 | 0.02122 |

### Example 4: Energy Bar Formulations with Designer-MNEI (DM) Proteins

Formulations of energy bar with DM proteins are presented in Table 8.

TABLE 8
DM31 based energy bar formulation (40% added sugar
reduction compared to energy bar without DM)

| Ingredients | Energy bar | |
|---|---|---|
| Whole oatmeal | 21-26 | g |
| Whole crisped rice | 17-22 | g |
| Pecans | 8-12 | g |
| Almonds | 8-12 | g |
| Glucose syrup | 17-22 | g |
| Palm oil | 6-9 | g |
| Glycerol | 1-3 | g |
| Soy lecithin | 0.2-1 | g |
| Salt | 0.1-0.5 | g |
| Maltodextrin | 2-5 | g |
| DM31 sweet protein | 7-10 | mg |

### Testing Methods—Amai's Expert Super-Taster Panel

All sensory evaluations were determined using a trained expert panel for analytical discrimination. The sensory expert panel was established by a screening process of potential tasters. Screening tests, conducted according to ISO standard (IS 8586-1), examined the sensory sensitivity, consistency, and sensory memory of the taster. The panel is well trained and calibrated. The selected panel is trained regularly to maintain high performance output.

Sensory Profiles for Energy Bar with DM Proteins Prototypes—Testing Procedure:

A sensory vocabulary was determined for the energy bar category by the expert panel. The sensory vocabulary was built by tasting a large range of products from the category and raising all the relevant sensory attributes which describes the category. Using as diverse a language as possible to best describe the products.

After having the sensory vocabulary, selecting the key attributes that will be used to describe the products in the questionnaire.

Building sensory profiles for energy bar with DM proteins: the panelists rated each tested product versus a reference on a two-way scale (between −3 to +3) with a fixed reference point (0) across all the attributes selected from the glossary. When the tested product was evaluated “more” than the reference on a specific attribute (e.g., sweeter, thicker, and so on), it got positive rates (+1, +2, or +3), and when it was rated “less” than the reference on a specific attribute (e.g., less sweet, less thick, and so on) it got negative rates (−1, −2 or −3). Before and between attributes, the tasters were requested to rinse their mouths with mineral water, eat an unsalted cracker and cucumber, and drink water again.

### Results

### Energy Bar Prototype

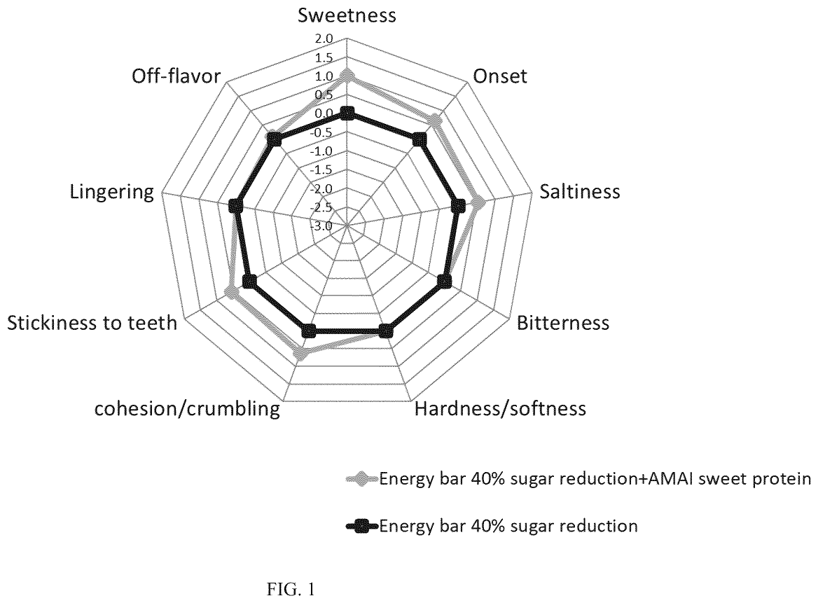

As demonstrated in FIG. 1, an energy bar with a 40% reduction of added sugar is less sweet compared to an energy bar with DM protein prototype.

### Example 5: Marzipan Formulation with Designer-MNEI (DM) Proteins

Marzipan is a confection made primary of grounded almonds and sugar. It is widely used in the baking industry and for the preparation of different kind of sweets.

Formulations of Marzipan with DM proteins are presented in Table 9.

TABLE 9
DM31 based Marzipan formulation (70% added sugar
reduction) compared to Marzipan without DM31.

| Ingredients | percentage |
|---|---|
| White Almonds | 28-48% |
| Maltodextrin | 20-40% |
| Sugar Powder | 3-23% |
| Water | 1-20% |
| Fibers | 1-10% |
| Glucose | 1-5% |
| Lemon Juice | 0.1-0.6% |
| Almond Extract | 0.1-0.6% |
| Sweet Protein DM-31 | 0.01-0.05% |

### Preparation Instructions:

Almonds were grinded, all the powders were added gradually to the mixer and grinded well. All the liquid ingredients as well as the sweet protein were added to the mass and continued grinding until a crystalline dough was obtained, 0.01-0.05% by weight of protein was added to the recipe. The sweet protein DM-31 potency is 1000-3000 and can be equivalent to 25-45 brix.

### Results

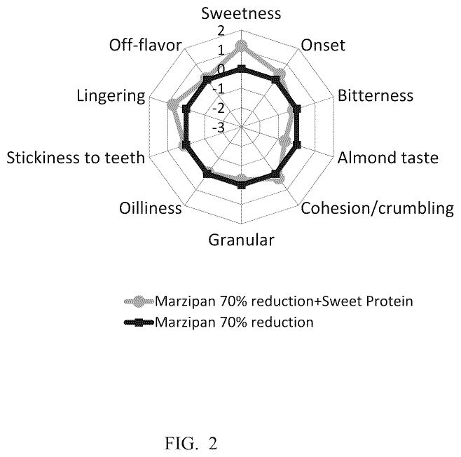

As demonstrated in FIG. 2, Marzipan with sweet protein DM-31 (70% reduction of added sugar) is sweeter than Marzipan with 70% reduction of added sugar.

### Example 6: Non-Dairy Milk Prototype with Designer-Monellin (DM) Proteins

Formulations of non-dairy milk with DM proteins are presented in Table 10.

TABLE 10
DM31 based non-dairy milk formulation 50% added sugar reduction
compared to non-dairy milk with 50% added sugar reduction
Formulations:

| Ingredients | Oat milk | |
|---|---|---|
| Oat milk no added sugar | 98-99.4 | g |
| Sugar | 0.6-2 | g |
| AMAI sweet protein DM-31 | 0.3-0.7 | mg |
| Ingredients | Almond milk | |
| Almond milk no added sugar | 98-99.4 | g |
| Sugar | 0.6-2 | g |
| AMAI sweet protein DM-31 | 0.6-1.4 | mg |
| Ingredients | Soy milk | |
| Soy milk no added sugar | 97.7-99.3 | g |
| Sugar | 0.7-2.3 | g |
| AMAI sweet protein DM-31 | 0.8-1.6 | mg |

### Preparation Instructions:

Sugar and AMAI sweet protein DM31 were added to the unsweetened non-dairy milk and mixes into a homogeneous solution.

### Results

Non-dairy milk with 50% added sugar reduction is less sweet compared to non-dairy milk with DM proteins prototype (50% added sugar reduction).

### Example 7: Granola Preparations

Formulations of granola with DM proteins are presented in Table 11.

TABLE 11
DM31 based granola

| Ingredients | Granola | |
|---|---|---|
| Whole oatmeal | 42-47 | g |
| Whole - rice crisped | 12-16 | g |
| Pecans | 9-12 | g |
| Almonds | 9-12 | g |
| Glucose syrup | 4-6 | g |
| powdered sugar (Sucrose) | 1-3 | g |
| Sunflower oil | 6-9 | g |
| Glycerol | 1-3 | g |
| lecithin | 0.2-1 | g |
| Salt | 0.05-0.2 | g |
| Maltodextrin | 2-5 | g |
| DM31 | 3-7 | mg |

### Preparation Instructions:

Oatmeal, rice crisped, pecans, and almonds are toasted for 15 minutes at 160° C.

Glucose syrup, powdered sugar, sunflower oil, glycerol, lecithin, and salt are heated to a homogenous syrup.

The toasted pecans and almonds are crushed to small pieces. The dry ingredients (oatmeal, crisped rice, pecans, and almonds) added to the syrup and mixed. Maltodextrin and DM31 are added at the end.

The mixture is poured into a baking dish.

The Granola mix is baked for 10 minutes at 90° C., then it is taken out, cooled down, and stored in a cool place in a closed container.

### Results:

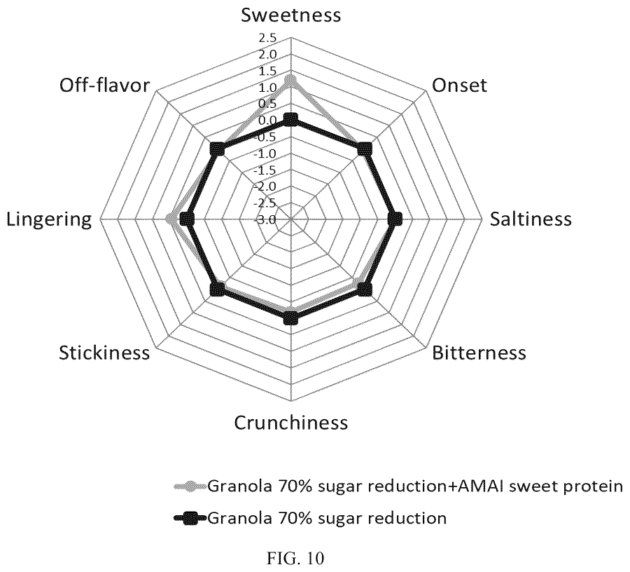

FIG. 10 the formulation of granola with 70% sugar reduction and AMAI sweet protein is sweeter, compared to granola with 70% sugar reduction and no-protein.

### Example 8: Peanut Butter Spread

Formulations of peanut butter spread for various filling options with DM proteins are presented in Table 12.

TABLE 12
DM31 based peanut butter spread.

| Material | percentage |
|---|---|
| Peanuts | 56-76% |
| Polydextrose | 5-15% |
| Sugar | 3-14 |
| Maltodextrin | 2-13% |
| Fibers | 1-11% |
| Palm oil | 1-5% |
| Salt | 0.1-0.5% |
| Sweet Protein DM-31 | 0.1-0.5% |

### Preparation Instructions:

1. Grind the peanuts.
2. Add gradually ingredients 2-7 to the mixer and grind well until homogeneous dough is obtained.
3. Add sweet protein DM-31 to the mass and continue grinding at slow rate to full assimilation.

### Results:

As shown in FIG. 11 in chocolate peanut butter cups, the peanut butter spread with 75% sugar reduction combined with Amai sweet protein is sweeter, compared to the peanut butter spread with 75% sugar reduction and no-protein addition.

### Example 9: Halva (Sesame) Spread

Formulations of halva (Sesame) spread with DM proteins are presented in Table 13.

TABLE 13
DM31 based halva (Sesame) spread

| | |
|---|---|
| Halva spread | Ingredients |

| | | |
|---|---|---|
| 62-67 | g | Raw Tahini |
| 4-7 | g | Powdered sugar |
| 23-27 | g | Maltodextrin |
| 2-5 | g | Sunflower oil |
| 0.2-0.8 | g | Soy lecithin |
| 5-8 | mg | DM31 |

### Preparation Instructions:

Mix all ingredients until a homogeneous mass is obtained.

Results: As shown in FIG. 20 the formulation of Halva spread with 80% sugar reduction and DM31 is sweeter than Halva spread with 80% sugar reduction without DM31.

### Preparation Instructions:

1. Mix all ingredients and grind them gradually in Chocolate Refiner for 6 hours until refined mass is obtained.
2. Tempering while stirring: first heat to 45° C., then cool to 27° C. and re-heat to 29° C.
3. Pour the chocolate into molds to set.

### Results:

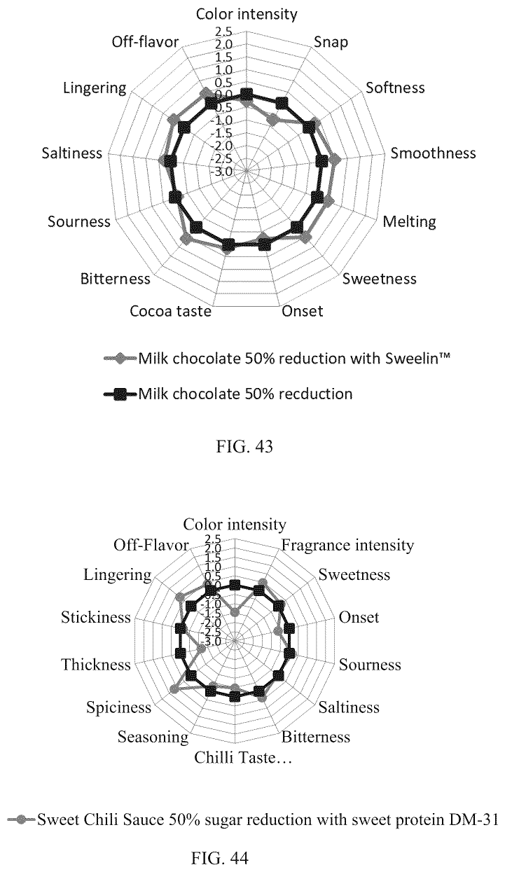

As demonstrated in FIG. 43, Milk Chocolate with 50% sugar reduction and sweet protein DM-31 is sweeter than Milk Chocolate with 50% sugar reduction.

### Example 11: Chocolate Peanut Butter Cups

The dessert is composed of two ingredients: Milk chocolate and Peanut butter spread that are combined.

Formulations of Milk Chocolate with DM proteins are presented in Table 15.

TABLE 15
DM 31 based Milk chocolate for peanut butter cups

| | Material | percentage |
|---|---|---|
| 1 | Chocolate 54.5% | 32-47% |
| 2 | Milk powder | 17%-33% |
| 3 | Cacao butter | 8%-16% |
| 4 | Cacao mass | 5%-13% |
| 5 | Fibers | 4%-12% |
| 6 | Whey | 1%-5% |
| 7 | Glucose | 1%-5% |
| 8 | Lecithin | 0.1%-0.7% |
| 9 | Sweet Protein DM-31 | 0.01-0.04% |

### Preparation Instructions:

1. Melt the chocolate with the cocoa mass and cocoa butter at 70 deg for 3-5 min.
2. Add glucose and fibers and mix at 70 deg for 1-3 minutes.
3. Add milk powder, whey and lecithin and mix at 75-80 deg for 10 minutes.
4. Cool the mass to 70 deg and add sweet protein DM-31, mix for 3-5 minutes to full assimilation.
5. Pour the chocolate into molds and cool the mass.

### Peanut Butter Spread

Peanut butter spread described above.

### Results:

As shown in FIG. 43, in chocolate peanut butter cups, the peanut butter spread with 75% sugar reduction combined with Amai sweet protein is sweeter, compared to the peanut butter spread with 75% sugar reduction and no-protein addition.

### Example 12: Sweet Chili Sauce

Formulations of Sweet chili sauce with DM proteins are presented in Table 16.

TABLE 16
DM 31 based sweet chili sauce (50% added sugar reduction)

| | Material | Percentage |
|---|---|---|
| 1 | Water | 56%-70% |
| 2 | Sugar | 12%-26% |
| 3 | Vinegar 5% | 1.8%-9.8% |
| 4 | Lemon juice | 1.5%-5.5% |
| 5 | Ground chili | 1.9%-3.9% |
| 6 | Salt | 0.5%-3.5% |
| 7 | Sweet Protein DM-31 | 0.5%-2.9% |
| 8 | Corn flour | 0.5%-1.5% |
| 9 | Garlic powder | 0.2%-1.4% |
| 10 | Xanthan gum | 0.1%-0.5% |

### Preparation Instructions:

1. Add vinegar, lemon juice, ground chili, garlic powder, salt and corn flour.
2. In a separate bowl, weigh sugar and xanthan gum and mix well.
3. Mix both bowls together and add water.
4. Cook for 10 minutes at 114° C.
5. Cook for another 30 minutes at 95° C.
6. Add Sweelin™ and mix well.
7. After cooling, pass through a fine sieve.

### Results:

As demonstrated in FIG. 44, Sweet chili sauce with 50% sugar reduction combined with Amai sweet protein is as sweet as a full sugar product.

### Example 13: Sugar Syrup for Savarina Cake

Formulations of sugar syrup with DM proteins are presented in Table 17.

TABLE 17
DM 31 based sugar syrup (50% added sugar reduction)

| Material | Percentage |
|---|---|
| Water | 82%-92% |
| Sugar | 6%-16% |
| Rose water | 0.5%-2.5% |
| Sweet Protein DM-31 | 0.3%-1% |
| Vanilla flavored extract | 0.1%-1% |

### Preparation Instructions:

1. Prepare savarina cake according to the manufacturer's instructions and let it cool completely.
2. Boil water, while mixing add sugar.
3. When boiled add rose water, vanilla-flavored extract, and sweet protein DM31 and mix well.
4. Pour into a bowl and soak the savarina cake for 10 min.
5. Serve cake with prepared whipped cream.

### Results:

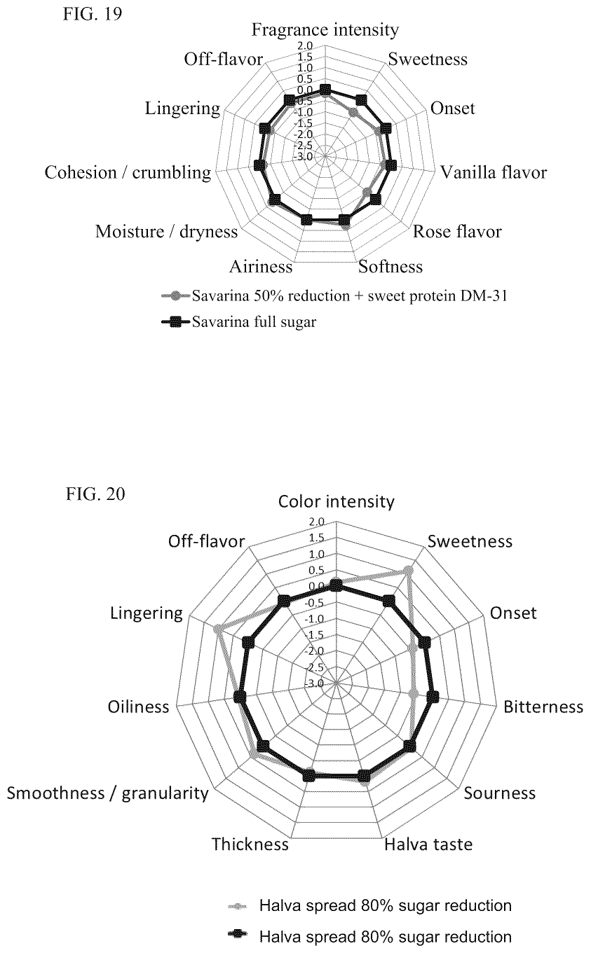

As demonstrated in FIG. 19, savarina with 50% sugar reduction combined with Amai sweet protein is as sweet as a full sugar product.

### Example 14: Vinaigrette Salad Dressing

Formulations of vinaigrette salad dressing with DM proteins are presented in Table 18.

TABLE 18
DM 31 based vinaigrette salad dressing
with 50% added sugar reduction.

| | Material | Percentage |
|---|---|---|
| 1 | Lemon juice | 29%-39% |
| 2 | Water | 26%-36% |
| 3 | Sugar | 8%-17% |
| 4 | Vegetable oil | 6%-16% |
| 5 | Fibers | 2%-11% |
| 6 | Mustard | 0.5%-7.5% |
| 7 | Salt | 0.5%-4% |
| 8 | Sweet Protein DM-31 | 0.3%-1% |
| 9 | Guar gum | 0.1%-0.5% |
| 10 | Xanthan gum | 0.1%-0.6% |

### Preparation Instructions:

1. Weigh all the ingredients except sweet protein.
2. Grind them all together in a blender for 1 minute.
3. Add sweet protein after blending and mix well.

### Results:

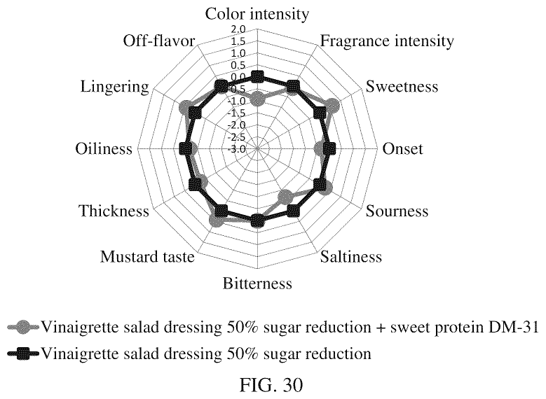

As demonstrated in FIG. 30, the vinaigrette with 50% sugar reduction combined with Amai sweet protein is sweeter, compared to vinaigrette with 50% sugar reduction and no-protein addition.

### Example 15: Teriyaki

Formulations of Teriyaki with DM proteins are presented in Table 19.

TABLE 19
DM 31 based teriyaki sauce with 70% added sugar reduction.

| Material | Percentage |
|---|---|
| Water | 55%-65% |
| Soy sauce | 18%-28% |
| Sugar | 8%-15% |
| Corn flour | 2%-5% |
| Glucose | 0.5%-4.5% |
| Sweet Protein DM-31 | 0.2%-1.2% |

### Preparation Instructions:

1. Mix corn flour with water until corn flour dissolves.
2. Weigh glucose, sugar, and soy sauce in a saucepan and add the corn flour with water.
3. Heat the mixture to 100° C. for 5 minutes. Then mix until the temperature lowers to 60° C. (about 10 minutes).
4. When the temperature reaches 60° C. add sweet protein DM-31.
5. Strain the sauce through a fine sieve.

### Results:

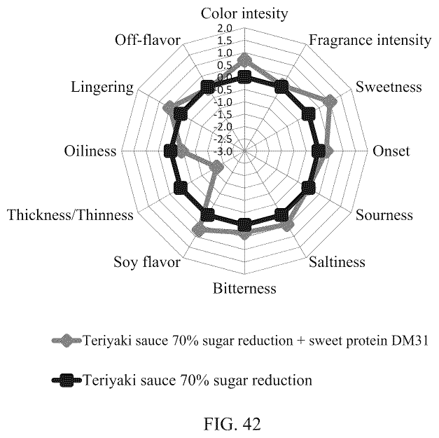

As demonstrated in FIG. 42, Teriyaki sauce with 70% sugar reduction combined with Amai sweet protein is sweeter, compared to Teriyaki sauce with 70% sugar reduction and no-protein addition.

### Example 16: Thousand Island Sauce

Formulations of Thousand Island sauce are presented in Table 20.

TABLE 20
DM 31 based thousand island sauce
with 62% added sugar reduction.

| Material | Percentage |
|---|---|
| Mayonnaise low fat | 30%-42% |
| Water | 25%-35% |
| Tomato paste (22 Bx) | 22%-32% |
| Fibers | 1%-8% |
| Sugar | 1%-5% |
| Vinegar | 0.6%-4.6% |
| Salt | 0.1%-0.9% |
| Xanthan | 0.1%-0.8% |
| Sweet Protein DM-31 | 0.1%-0.5% |
| Ketchup spice | 0.01%-0.05% |

1. Weigh tomato paste, ketchup spice, fibers, salt, sugar, vinegar, water, and xanthan.
2. Mix for 5 minutes, then 5 min in opposite direction until smooth mixture.
3. Check if all the xanthan dissolves. If not, repeat the mixing procedure.
4. After the mixture is homogeneous, add Sweet Protein DM-31 and mix slowly.
5. Add mayonnaise and water and mix well.

### Results:

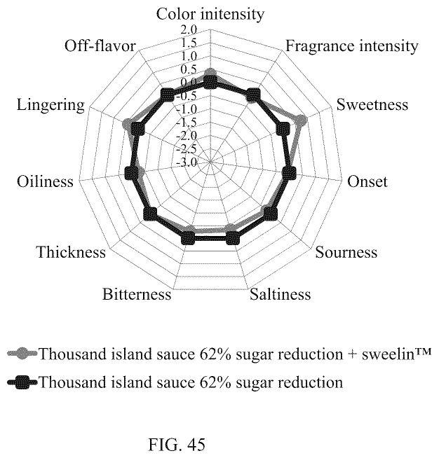

As shown in FIG. 45, Thousand island sauce with 62% sugar reduction combined with Amai sweet protein is sweeter, compared to thousand island sauce with 62% sugar reduction and no-protein addition.

### Example 17: Dark Chocolate

Formulations of dark chocolate are presented in Table 21.

### Preparation Instructions

1. Mix all ingredients and grind them in Chocolate Refiner for 4 hours until homogeneous mass is obtained.
2. Tempering while stirring: first heat to 45° C., then cool to 27° C. and re-heat to 31° C.
3. Pour the chocolate into molds to set.

### Results:

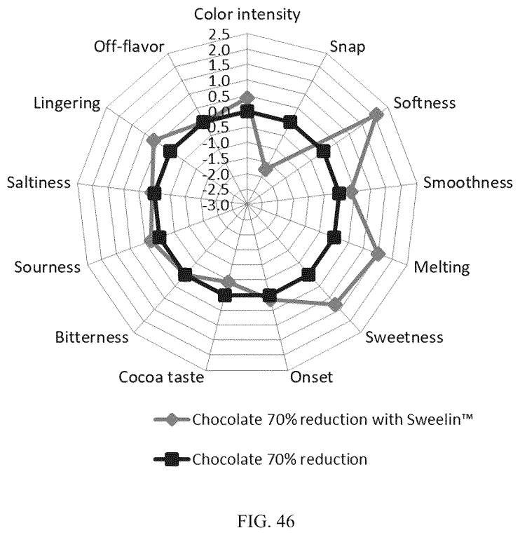

As shown in FIG. 46, the formulation of Dark Chocolate with 70% sugar reduction and Sweet protein DM-31 is sweeter than Dark Chocolate with 70% sugar reduction.

1. A modified protein comprising an amino acid sequence that has amino acid deletions in at least three amino acids located between amino acid T46 and amino acid 156, as compared to a reference protein, wherein said reference protein has an amino acid sequence set forth in SEQ ID NO:8.

2. The modified protein of claim 1, wherein said amino acid deletions comprises deletion of amino acids E50, F52 and R53 as compared to said reference protein.

3. The modified protein of claim 1, comprising amino acid substitutions in at least five amino acids as compared to the reference protein.

4. The modified protein of claim 3, wherein said amino acid substitutions comprise (i) substitutions in amino acids, E2, E23 and Y65 as compared to the reference protein or (ii) substitution in L70 as compared to said reference protein.

5. (canceled)

6. The modified protein of claim 3, wherein said amino acid substitution comprise (i) a substitution in at least one amino acid selected from the group consisting of E4, T12, A19, V20, K25, 126, Q28, R31, T33, N35, C41, Q61, V64, D68, A73, 175, R84 and F89 as compared to said reference protein, (ii) a substitution in at least one amino acid selected from the group consisting of A19, V20, K25, 126, Q28, T33, C41 and D68, as compared to said reference protein, (iii) a substitution in at least one amino acid selected from the group consisting of A19, V20, K25, 126, Q28, T33 and D68, as compared to said reference protein, (iv) an amino acid substitution in at least one amino acid selected from the group consisting of Q28, C41 and Q68 as compared to said reference protein, (v) a substitution in at least one amino acid selected from the group consisting of A19 and V20, as compared to said reference protein, (vi) a substitution in at least one amino acid selected from the group consisting of K25, 126 and Q28, as compared to said reference protein, or (vii) a substitution in at least one amino acid selected from the group consisting of T33 and D68, as compared to said reference protein.

7.-12. (canceled)

13. The modified protein of claim 3, wherein said amino acid substitution comprises amino substitutions E2N, E23A, Y65R and L70I.

14. The modified protein of claim 6, comprising (i) at least one amino acid substitution selected from the group consisting of E4Q, T12V, A19V, V20I, K25R, I26S, I26T, I26W, Q28K, Q28R, Q28K, Q28E, Q28S, R31T, T33R, N35T, C41T, C41V, C41A, C41S, Q61N, V64I, D68S, D68N, D68T, A73V, A73F, I75L, R84L, F89V, and F89M as compared to the reference protein, (ii) at least one amino acid substitution selected from the group consisting of A19V, V20I, K25R, I26S, I26T, I26W, Q28K, Q28R, Q28K, Q28E, Q28S, T33R, N35T, C41T, C41V, C41A, C41S, D68S, D68N, and D68T, as compared to the reference protein, or (iii) at least one amino acid substitution selected from the group consisting of A19V, V20I, K25R, Q28K, T33R, C41A, C41S, and D68N, as compared to the reference protein.

15.-16. (canceled)

17. The modified protein of claim 1, comprising an amino acid sequence selected from the group consisting of S SEQ ID NO:1, SEQ ID NO:2, SEQ ID NO:3, SEQ ID NO:4, SEQ ID NO:5, SEQ ID NO:6, SEQ ID NO:12, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:50, SEQ ID NO:51, SEQ ID NO:52, SEQ ID NO:54, SEQ ID NO:56, SEQ ID NO:57, SEQ ID NO:58, SEQ ID NO:59, SEQ ID NO: 60, SEQ ID NO:63, SEQ ID NO:64, SEQ ID NO:65, SEQ ID NO:66, SEQ ID NO:67, SEQ ID NO:68, SEQ ID NO:69 and SEQ ID NO:71 or of a fragment or variant thereof.

18. The modified protein of claim 1, comprising an amino acid sequence selected from the group consisting of SEQ ID NO:5, SEQ ID NO:14, SEQ ID NO:17, SEQ ID NO:25, SEQ ID NO:34, SEQ ID NO:36, SEQ ID NO:38 and SEQ ID NO:49, or of a fragment or variant thereof.

19. The modified protein of claim 1 having at least one improved property as compared to said reference protein, said at least one improved property is selected from the group consisting of better sweetness profile, shortened sweet taste lingering, better sweetness potency, better sweetness kinetics, increased thermal stability, high pressure stability, increased pH stability, decreased binding to hydrophobic regions, improved freeze-thaw stability, improved reconstitutability after drying, increased solubility, a sensory profile that is closer to that of sugar and increased shelf-life stability, optionally wherein said improved property is sweetness profile and stability.

20. (canceled)

21. The modified protein of claim 1 or any combination of two or more of said protein, for use in the preparation of a product for oral delivery.

22. The modified protein of claim 21, wherein the product is a food product, a food supplementary product, or a medicament.

23. The modified protein of claim 1 or any combination of two or more of said protein, for use as a flavor modifying agent, a flavor enhancing agent or a flavor masking agent.

24. The modified protein of claim 1 or any combination of two or more of said modified protein, for use as a sweetener.

25. A food product comprising the modified protein of claim 1, or any combinations thereof.

26. The food product of claim 25, being (i) a reduced sugar or no-sugar added food product, (ii) a beverage selected from the group consisting of a carbonated soft drink, a non-carbonated soft drink, a fountain beverage, a frozen ready-to-drink beverage, a coffee beverage, a tea beverage, a dairy beverage, a non-dairy milk, a fruit beverage, a flavored water, an enhanced water, a sports drink, an energy drink, an isotonic drink, low-calorie drink, and an alcoholic beverage, optionally wherein said non-dairy milk is selected from the group consisting of almond milk, cashew milk, soy milk, coconut milk, pea milk, macadamia milk, tiger nut milk, chickpea milk, rice milk, oat milk and flax milk.

27.-28. (canceled)

29. The food product of claim 25, being a food selected from the group consisting of (i) bakery products, cookies, biscuits, baking mixes, cereals, energy bars, marzipans, confectioneries, candies, toffees, chewing gum, bubble gum, dairy products, yogurts, flavored yogurts, peanut butter, soy sauce, soy base products, non-dairy products, salad dressings, ketchup, mayonnaise, vinegar, frozen-desserts, meat products, fish-meat products, bottled and canned foods, tabletop sweeteners, chocolate, fruits, dry fruits, and vegetables or (ii) an energy bar and marzipan.

30. (canceled)

31. The food product of claim 25, comprising at least one food ingredient.

32. The food product of claim 31, wherein the food ingredient is (i) at least one of artificial flavor, food additive, food coloring, preservative, or a sweetness enhancer, (ii) selected from the group consisting of stevia, sucrose, agave nectar, brown rice syrup, date sugar, honey, maple syrup, molasses, monk fruit, sugar alcohols, rare sugars, aspartame, sucralose, acesulfame potassium, saccharin, neotame, advantame, and dietary fibers, or (iii) selected from the group consisting of a flavor, food additive, food coloring, preservative, and sweetness enhancer.

33.-34. (canceled)

35. A sweetening composition comprising the protein of claim 1 or any combination thereof.


SEQUENCE LISTING (65 sequences)

SEQ ID NO: 1 - protein, 93 aa, synthetic construct
  source 1..93: note = Synthetic protein DM46
GNWEIIDIGPFTQNLGKFAVDEANKIGQYGRLTFNKVIRPTMKKTIYENGEIKGYEYQLYVRASDKIFRADISEDYKTRGRKLLRFNGPVPPP

SEQ ID NO: 2 - protein, 93 aa, synthetic construct
  source 1..93: note = Synthetic protein DM47
GNWEIIDIGPFTQNLGKFAVDEANKIGQYGTLTFNKVIRPCMKKTIYENGEIKGYEYQLYVRASDKIFRADISEDYKTRGRKLLRFNGPVPPP

SEQ ID NO: 3 - protein, 93 aa, synthetic construct
  source 1..93: note = Synthetic protein DM70
GNWEIIDIGPFTQNLGKFAVDEANKIGQYGRLTFNKVIRPCMKKTIYENGEIKGYEYQLYVRASDKIFRADISEDYKTRGLKLLRFNGPVPPP

SEQ ID NO: 4 - protein, 93 aa, synthetic construct
  source 1..93: note = Synthetic protein DM42
GNWEIIDIGPFTQNLGKFAVDEANKTGQYGRLTFNKVIRPCMKKTIYENGEIKGYEYQLYVRASDKIFRADISEDYKTRGRKLLRFNGPVPPP

SEQ ID NO: 5 - protein, 93 aa, synthetic construct
  source 1..93: note = Synthetic protein DM43
GNWEIIDIGPFTQNLGKFAVDEANRIGQYGRLTFNKVIRPCMKKTIYENGEIKGYEYQLYVRASDKIFRADISEDYKTRGRKLLRFNGPVPPP

SEQ ID NO: 6 - protein, 93 aa, synthetic construct
  source 1..93: note = Synthetic protein DM72
GNWEIIDIGPFTQNLGKFAVDEANKIGQYGRLTFTKVIRPCMKKTIYENGEIKGYEYQLYVRASDKIFRADISEDYKTRGRKLLRFNGPVPPP

SEQ ID NO: 7 - protein, 93 aa, synthetic construct
  source 1..93: note = Synthetic protein DM31
GNWEIIDIGPFTQNLGKFAVDEANKIGQYGRLTFNKVIRPCMKKTIYENGEIKGYEYQLYVRASDKIFRADISEDYKTRGRKLLRFNGPVPPP

SEQ ID NO: 8 - protein, 96 aa, synthetic construct [sequence omitted]

SEQ ID NO: 9 - protein, 96 aa, synthetic construct
  source 1..96: note = Synthetic protein DM09
GNWEIIDIGPFTQNLGKFAVDEANKIGQYGRLTFNKVIRPCMKKTIYENEGFREIKGYEYQLYVRASDKLFRADISEDYKTRGRKLLRFNGPVPPP

SEQ ID NO: 10 - protein, 96 aa, synthetic construct
  source 1..96: note = Synthetic protein DM14
GNWEIIDIGPFTQNLGKFAVDEANKIGQYGRLTFNKVIRPCMKKTIYENEGFREIKGYEYQLYVRASDKIFRADISEDYKTRGRKLLRFNGPVPPP

SEQ ID NO: 11 - protein, 93 aa, synthetic construct
  source 1..93: note = Synthetic protein DM29
GEWEIIDIGPFTQNLGKFAVDEENKIGQYGRLTFNKVIRPCMKKTIYENGEIKGYEYQLYVYASDKLFRADISEDYKTRGRKLLRFNGPVPPP

SEQ ID NO: 12 - protein, 93 aa, synthetic construct
  source 1..93: note = Synthetic protein DM41
GNWEIIDIGPFTQNLGKFAVDEANKSGQYGRLTFNKVIRPCMKKTIYENGEIKGYEYQLYVRASDKIFRADISEDYKTRGRKLLRFNGPVPPP

SEQ ID NO: 13 - protein, 96 aa, synthetic construct
  source 1..96: note = Synthetic protein DM42b
GEWEIIDIGPFTQNLGKFAVDEENKTGQYGRLTFNKVIRPCMKKTIYENEGFREIKGYEYQLYVYASDKLFRADISEDYKTRGRKLLRFNGPVPPP

SEQ ID NO: 14 - protein, 93 aa, synthetic construct
  source 1..93: note = Synthetic protein DM65
GNWEIIDIGPFTQNLGKFAVDEANKIGKYGRLTFNKVIRPCMKKTIYENGEIKGYEYQLYVRASDKIFRADISEDYKTRGRKLLRFNGPVPPP

SEQ ID NO: 15 - protein, 93 aa, synthetic construct
  source 1..93: note = Synthetic protein DM66
GNWEIIDIGPFTQNLGKFAVDEANKIGRYGRLTFNKVIRPCMKKTIYENGEIKGYEYQLYVRASDKIFRADISEDYKTRGRKLLRFNGPVPPP

SEQ ID NO: 16 - protein, 93 aa, synthetic construct
  source 1..93: note = Synthetic protein DM68
GNWEIIDIGPFTQNLGKFAVDEANRIGRYGRLTFNKVIRPCMKKTIYENGEIKGYEYQLYVRASDKIFRADISEDYKTRGRKLLRFNGPVPPP

SEQ ID NO: 17 - protein, 93 aa, synthetic construct
  source 1..93: note = Synthetic protein DM69
GNWEIIDIGPFTQNLGKFAVDEANRIGKYGRLTFNKVIRPCMKKTIYENGEIKGYEYQLYVRASDKIFRADISEDYKTRGRKLLRFNGPVPPP

SEQ ID NO: 18 - protein, 93 aa, synthetic construct
  source 1..93: note = Synthetic protein DM73
GNWEIIDIGPFTQNLGKFAVDEANKIGRYGTLTFNKVIRPCMKKTIYENGEIKGYEYQLYVRASDKIFRADISEDYKTRGRKLLRFNGPVPPP

SEQ ID NO: 19 - protein, 93 aa, synthetic construct
  source 1..93: note = Synthetic protein DM92
GNWEIIDIGPFTQNLGKFAVDEANKTGKYGRLTFNKVIRPCMKKTIYENGEIKGYEYQLYVRASDKIFRADISEDYKTRGRKLLRFNGPVPPP

SEQ ID NO: 20 - protein, 93 aa, synthetic construct
  source 1..93: note = Synthetic protein DM103
GNWEIIDIGPFTQNLGKFAVDEANKIGQYGRLTFNKVIRPCMKKTIYENGEIKGYEYQLYVRASDKIFRADLSEDYKTRGRKLLRFNGPVPPP

SEQ ID NO: 21 - protein, 93 aa, synthetic construct
  source 1..93: note = Synthetic protein DM115
GNWEIIDIGPFTQNLGKFAVDEANKWGEYGRLTFNKVIRPCMKKTIYENGEIKGYEYQLYVRASDKIFRADISEDYKTRGRKLLRFNGPVPPP

SEQ ID NO: 22 - protein, 93 aa, synthetic construct
  source 1..93: note = Synthetic protein DM116
GNWEIIDIGPFTQNLGKFAVDEANKWGKYGRLTFNKVIRPCMKKTIYENGEIKGYEYQLYVRASDKIFRADISEDYKTRGRKLLRFNGPVPPP

SEQ ID NO: 23 - protein, 93 aa, synthetic construct
  source 1..93: note = Synthetic protein DM144
GNWEIIDIGPFTQNLGKFAVDEANKWGQYGRLTFNKVIRPCMKKTIYENGEIKGYEYQLYVRASDKIFRADISEDYKTRGRKLLRFNGPVPPP

SEQ ID NO: 24 - protein, 93 aa, synthetic construct
  source 1..93: note = Synthetic protein DM145
GNWEIIDIGPFTQNLGKFAVDEANKIGEYGRLTFNKVIRPCMKKTIYENGEIKGYEYQLYVRASDKIFRADISEDYKTRGRKLLRFNGPVPPP

SEQ ID NO: 25 - protein, 93 aa, synthetic construct
  source 1..93: note = Synthetic protein DM77
GNWEIIDIGPFTQNLGKFVVDEANKIGQYGRLTFNKVIRPCMKKTIYENGEIKGYEYQLYVRASDKIFRADISEDYKTRGRKLLRFNGPVPPP

SEQ ID NO: 26 - protein, 93 aa, synthetic construct
  source 1..93: note = Synthetic protein DM84
GNWEIIDIGPFTQNLGKFAVDEANKIGQYGRLTFNKVIRPCMKKTIYENGEIKGYEYQLYIRASDKIFRADISEDYKTRGRKLLRFNGPVPPP

SEQ ID NO: 27 - protein, 93 aa, synthetic construct
  source 1..93: note = Synthetic protein DM85
GNWEIIDIGPFTQNLGKFAVDEANKIGQYGRLTFNKVIRPCMKKTIYENGEIKGYEYQLYVRASDKIFRVDISEDYKTRGRKLLRFNGPVPPP

SEQ ID NO: 28 - protein, 93 aa, synthetic construct
  source 1..93: note = Synthetic protein DM91
GNWEIIDIGPFTQNLGKFAVDEANKIGQYGRLTFNKVIRPVMKKTIYENGEIKGYEYQLYVRASDKIFRADISEDYKTRGRKLLRFNGPVPPP

SEQ ID NO: 29 - protein, 93 aa, synthetic construct
  source 1..93: note = Synthetic protein DM104
GNWEIIDIGPFTQNLGKFAVDEANKIGQYGRLTFNKVIRPCMKKTIYENGEIKGYEYQLYVRASDKIFRADISEDYKTRGRKLLRMNGPVPPP

SEQ ID NO: 30 - protein, 93 aa, synthetic construct
  source 1..93: note = Synthetic protein DM131
GNWEIIDIGPFTQNLGKFAVDEANKIGQYGRLTFNKVIRPCMKKTIYENGEIKGYEYQLYVRASDKIFRFDISEDYKTRGRKLLRFNGPVPPP

SEQ ID NO: 31 - protein, 93 aa, synthetic construct
  source 1..93: note = Synthetic protein DM132
GNWEIIDIGPFTQNLGKFAVDEANKIGQYGRLTFNKVIRPCMKKTIYENGEIKGYEYQLYVRASDKIFRFDISEDYKTRGRKLLRMNGPVPPP

SEQ ID NO: 32 - protein, 93 aa, synthetic construct
  source 1..93: note = Synthetic protein DM150
GNWEIIDIGPFTQNLGKFAVDEANKIGQYGRLTFNKVIRPAMKKTIYENGEIKGYEYQLYVRASDKIFRADISEDYKTRGRKLLRFNGPVPPP

SEQ ID NO: 33 - protein, 93 aa, synthetic construct
  source 1..93: note = Synthetic protein DM151
GNWEIIDIGPFTQNLGKFAVDEANKIGQYGRLTFNKVIRPSMKKTIYENGEIKGYEYQLYVRASDKIFRADISEDYKTRGRKLLRFNGPVPPP

SEQ ID NO: 34 - protein, 93 aa, synthetic construct
  source 1..93: note = Synthetic protein DM57
GNWEIIDIGPFTQNLGKFAVDEANKIGQYGRLRFNKVIRPCMKKTIYENGEIKGYEYQLYVRASDKIFRADISEDYKTRGRKLLRFNGPVPPP

SEQ ID NO: 35 - protein, 93 aa, synthetic construct
  source 1..93: note = Synthetic protein DM75
GNWQIIDIGPFTQNLGKFAVDEANKIGQYGRLTFNKVIRPCMKKTIYENGEIKGYEYQLYVRASDKIFRADISEDYKTRGRKLLRFNGPVPPP

SEQ ID NO: 36 - protein, 93 aa, synthetic construct
  source 1..93: note = Synthetic protein DM87
GNWEIIDIGPFTQNLGKFAVDEANKIGQYGRLTFNKVIRPCMKKTIYENGEIKGYEYQLYVRASNKIFRADISEDYKTRGRKLLRFNGPVPPP

SEQ ID NO: 37 - protein, 93 aa, synthetic construct
  source 1..93: note = Synthetic protein DM93
GNWEIIDIGPFTQNLGKFAVDEANKIGQYGRLTFNKVIRPCMKKTIYENGEIKGYEYQLYVRASNKIFRADISEDYKTRGLKLLRFNGPVPPP

SEQ ID NO: 38 - protein, 93 aa, synthetic construct
  source 1..93: note = Synthetic protein DM96
GNWEIIDIGPFTQNLGKFAIDEANKIGQYGRLTFNKVIRPCMKKTIYENGEIKGYEYQLYVRASDKIFRADISEDYKTRGRKLLRFNGPVPPP

SEQ ID NO: 39 - protein, 96 aa, synthetic construct
  source 1..96: note = Synthetic protein DM157
GEWEIIDIGPFTQNLGKFAVDEENKIGQYGRLTFNKVIRPCMKKTIYENEGFREIKGYEYQLYVYASNKLFRADISEDYKTRGRKLLRFNGPVPPP

SEQ ID NO: 40 - protein, 93 aa, synthetic construct
  source 1..93: note = Synthetic protein DM28
GNWEIIDIGPFTQNLGKFAVDEANKIGQYGRLTFNKVIRPCMKKTIYENGEIKGYEYQLYVRASDKLFRADISEDYKTRGRKLLRFNGPVPPP

SEQ ID NO: 41 - protein, 93 aa, synthetic construct
  source 1..93: note = Synthetic protein DM94
GNWEIIDIGPFTQNLGKFAVDEANKIGQYGTLTFNKVIRPCMKKTIYENGEIKGYEYQLYVRASNKIFRADISEDYKTRGRKLLRFNGPVPPP

SEQ ID NO: 42 - protein, 93 aa, synthetic construct
  source 1..93: note = Synthetic protein DM97
GNWEIIDIGPFTQNLGKFAVDEANKIGQYGTLTFNKVIRPCMKKTIYENGEIKGYEYQLYVRASDKIFRADISEDYKTRGLKLLRFNGPVPPP

SEQ ID NO: 43 - protein, 93 aa, synthetic construct
  source 1..93: note = Synthetic protein DM99
GNWEIIDIGPFTQNLGKFAVDEANKIGQYGRLTFNKVIRPCMKKTIYENGEIKGYEYNLYVRASDKIFRADISEDYKTRGRKLLRFNGPVPPP

SEQ ID NO: 44 - protein, 93 aa, synthetic construct
  source 1..93: note = Synthetic protein DM143
GNWEIIDIGPFVQNLGKFAVDEANKIGQYGRLTFNKVIRPVMKKTIYENGEIKGYEYQLYVRASDKIFRADISEDYKTRGRKLLRFNGPVPPP

SEQ ID NO: 45 - protein, 93 aa, synthetic construct
  source 1..93: note = Synthetic protein DM152
GNWEIIDIGPFVQNLGKFAVDEANKIGQYGRLTFNKVIRPCMKKTIYENGEIKGYEYQLYVRASDKIFRADISEDYKTRGRKLLRFNGPVPPP

SEQ ID NO: 46 - protein, 93 aa, synthetic construct
  source 1..93: note = Synthetic protein DM32
GNWEIIDIGPFTQNLGKFAVDEVNKIGQYGRLTFNKVIRPCMKKTIYENGEIKGYEYQLYVKASDKIFRADISEDYKTRGRKLLRFNGPVPPP

SEQ ID NO: 47 - protein, 93 aa, synthetic construct
  source 1..93: note = Synthetic protein DM33
GNWEIIDIGPFTQNLGKFAVDEVNKIGQYGRLTFNTVIRPCMKKTIYENGEIKGYEYQLYVKASDKIFRADISEDYKTRGRKLLRFNGPVPPP

SEQ ID NO: 48 - protein, 93 aa, synthetic construct
  source 1..93: note = Synthetic protein DM61
GNWEIIDIGPFTQNLGKFAVDEANKIGQYGRLTFNKVIRPCMKKTIYENGEIKGYEYQLYVRASSKIFRADISEDYKTRGRKLLRFNGPVPPP

SEQ ID NO: 49 - protein, 93 aa, synthetic construct
  source 1..93: note = Synthetic protein DM108
GNWEIIDIGPFTQNLGKFAVDEANKIGQYGRLTFNKVIRPCMKKTIYENGEIKGYEYQLYVRASTKIFRADISEDYKTRGRKLLRFNGPVPPP

SEQ ID NO: 50 - protein, 93 aa, synthetic construct
  source 1..93: note = Synthetic protein DM117
GNWEIIDIGPFTQNLGKFAIDEANKIGQYGRLTFNKVIRPCMKKTIYENGEIKGYEYQLYIRASDKIFRADISEDYKTRGRKLLRFNGPVPPP

SEQ ID NO: 51 - protein, 93 aa, synthetic construct
  source 1..93: note = Synthetic protein DM330
GNWEIIDIGPFTQNLGKFAVDEANKIGSYGRLTFNKVIRPCMKKTIYENGEIKGYEYQLYVRASDKIFRADISEDYKTRGRKLLRFNGPVPPP

SEQ ID NO: 52 - protein, 93 aa, synthetic construct
  source 1..93: note = Synthetic protein DM464
GNWEIIDIGPFTQNLGKFAVDEANRIGQYGRLTFNKVIRPCMKKTIYENGEIKGYEYQLYVRASNKIFRADISEDYKTRGRKLLRFNGPVPPP

SEQ ID NO: 53 - protein, 93 aa, synthetic construct
  source 1..93: note = Synthetic protein DM491
GNWEIIDIGPFTQNLGKFAIDEANKIGQYGRLTFNKVIRPAMKKTIYENGEIKGYEYQLYVRASDKIFRFDLSEDYKTRGRKLLRMNGPVPPP

SEQ ID NO: 54 - protein, 93 aa, synthetic construct
  source 1..93: note = Synthetic protein DM498
GNWEIIDIGPFTQNLGKFAVDEANKIGQYGRLTFNKVIRPSMKKTIYENGEIKGYEYQLYIRASDKIFRFDLSEDYKTRGRKLLRVNGPVPPP

SEQ ID NO: 55 - protein, 93 aa, synthetic construct
  source 1..93: note = Synthetic protein DM506
GNWEIIDIGPFTQNLGKFAIDEANKIGQYGRLTFNKVIRPCMKKTIYENGEIKGYEYQLYVRASNKIFRADISEDYKTRGRKLLRFNGPVPPP

SEQ ID NO: 56 - protein, 93 aa, synthetic construct
  source 1..93: note = Synthetic protein DM508
GNWEIIDIGPFTQNLGKFAIDEANKIGKYGRLTFNKVIRPCMKKTIYENGEIKGYEYQLYVRASDKIFRADISEDYKTRGRKLLRFNGPVPPP

SEQ ID NO: 57 - protein, 93 aa, synthetic construct
  source 1..93: note = Synthetic protein DM509
GNWEIIDIGPFTQNLGKFAIDEANRIGQYGRLTFNKVIRPCMKKTIYENGEIKGYEYQLYVRASDKIFRADISEDYKTRGRKLLRFNGPVPPP

SEQ ID NO: 58 - protein, 93 aa, synthetic construct
  source 1..93: note = Synthetic protein DM341
GNWEIIDIGPFTQNLGKFAVDEANKIGQYGRLTFNTVIRPCMKKTIYENGEIKGYEYQLYVRASDKIFRADISEDYKTRGRKLLRFNGPVPPP

SEQ ID NO: 59 - protein, 93 aa, synthetic construct
  source 1..93: note = Synthetic protein DM420
GNWEIIDIGPFTQNLGKFAVDEANKIGQYGRLTFNKVIRPCMKKTIYENGEIKGYEYQLYVRASDKIFRADISEDYKTRGYKLLRFNGPVPPP

SEQ ID NO: 60 - protein, 93 aa, synthetic construct
  source 1..93: note = Synthetic protein DM424
GNWEIIDIGPFTQNLGKFAVDEANKIGQYGRLTFNKVIRPCMKKTIYENGEIKGYEYQLYVRASDKIFRADISEDYKTRGIKLLRFNGPVPPP

SEQ ID NO: 61 - protein, 93 aa, synthetic construct
  source 1..93: note = Synthetic protein DM432
GNWEIIDIGPFTQNLAKFAVDEANKIGQYGRLTFNKVIRPAMKKTIYENGEIKGYEYQLYLRASDKIFRADVSEDYKTRGRKLLRFNGPVPPP

SEQ ID NO: 62 - protein, 93 aa, synthetic construct
  source 1..93: note = Synthetic protein DM452
GNWEIIDIGPFTQNLGKFAVNEANKIGQYGRLTFNKVIRPCMKKTIYENGEIKGYEYQLYVRASDKIFRADISEDYKTRGRKLLRFNGPVPPP

SEQ ID NO: 63 - protein, 93 aa, synthetic construct
  source 1..93: note = Synthetic protein DM489
GNWEIIDIGPFTQNLGKFAIDEANKIGQYGRLTFNKVIRPVMKKTIYENGEIKGYEYQLYVRASDKIFRVDISEDYKTRGRKLLRFNGPVPPP

SEQ ID NO: 64 - protein, 93 aa, synthetic construct
  source 1..93: note = Synthetic protein DM505
GNWEIIDIGPFTQNLGKFVVDEANKIGKYGRLTFNKVIRPCMKKTIYENGEIKGYEYQLYVRASDKIFRADISEDYKTRGRKLLRFNGPVPPP

SEQ ID NO: 65 - protein, 93 aa, synthetic construct
  source 1..93: note = Synthetic protein DM510
GNWEIIDIGPFTQNLGKFVVDEANRIGQYGRLTFNKVIRPCMKKTIYENGEIKGYEYQLYVRASDKIFRADISEDYKTRGRKLLRFNGPVPPP